(19) United States
(12) Patent Application Publication
(10) Pub. No.: US 2023/0099384 A1
(43) Pub. Date: Mar. 30, 2023

(54) Genomics-Assisted Prediction Method for Apple Fruit Quality Traits and Disease Resistance and Use Thereof

(71) Applicant: China Agricultural University, Beijing (CN)

(72) Inventors: Xinzhong Zhang, Beijing (CN); Fei Shen, Beijing (CN); Bei Wu, Beijing (CN); Zhenhai Han, Beijing (CN); Xuefeng Xu, Beijing (CN); Yi Wang, Beijing (CN); Ting Wu, Beijing (CN); Wei Li, Beijing (CN); Changpeng Qiu, Beijing (CN); Xi Zhang, Beijing (CN)

(21) Appl. No.: 17/579,041

(22) Filed: Jan. 19, 2022

(30) Foreign Application Priority Data
Aug. 23, 2021 (CN) 202110965711.2

(51) Int. Cl. C12N 15/82; C12Q 1/6895

(52) U.S. Cl. CPC C12N 15/8262; C12N 15/8282; C12Q 1/6895; C12Q 2600/13; C12Q 2600/158

(57) ABSTRACT

The present disclosure provides a genomics-assisted prediction method for apple fruit quality traits and disease resistance and use thereof, and belongs to the technical field of plant genetics, genomics and breeding. The method is achieved by molecular markers. There are a total of 319 molecular markers, including 318 single-nucleotide polymorphism (SNP) markers and 1 InDel marker; and the molecular markers are associated with 16 traits of the Malus genus, including fruit ripening date, fruit shape, fruit cover color degree, fruit weight, soluble solid content, fruit juice pH value, malate content, chlorogenate content, procyanidin B2 content, flesh firmness at harvest, flesh crispness at harvest, flesh firmness retainability, flesh crispness retainability, fruit ring rot disease resistance, Glomerella leaf blotch resistance, and spur tree architecture. The molecular markers of the present disclosure can be used for apple germplasm resource evaluation and breeding, and can greatly improve apple breeding efficiency and shorten breeding cycle.

### CROSS REFERENCE TO RELATED APPLICATION(S)

This patent application claims the benefit and priority of Chinese Patent Application No. 202110965711.2, filed on Aug. 23, 2021, the disclosure of which is incorporated by reference herein in its entirety as part of the present application.

### REFERENCE TO AN ELECTRONIC SEQUENCE LISTING

The contents of the electronic sequence listing (SEQUENCE LISTING.txt;’ Size: 125 KB; and Date of Creation: Nov. 23, 2021) are herein incorporated by reference in its entirety.

### TECHNICAL FIELD

The present disclosure relates to the technical field of plant molecular breeding, in particular to a genomics-assisted prediction method for apple fruit quality traits and disease resistance and use thereof.

### BACKGROUND ART

Apple is one of the important economic crops over the world. The genetic improvement and diversification of apple cultivars is urgently needed to ensure the healthy development of the apple industry.

For a long time, in apple breeding, old cultivars are improved or new cultivars are bred mainly by cross breeding and bud mutation selection. However, it is difficult to improve the breeding efficiency of apple due to the long breeding cycle, highly-heterozygous genotypes, self-incompatibility, and lack of reliable early selection methods. With the development of modern breeding technology, molecular breeding technologies represented by marker assisted selection (MAS) and genomic selection (GS) have been established and gradually applied to breeding practices. These technologies provide a feasible technical solution for early selection of traits, shortening the generation interval, reducing the population size and improving breeding accuracy. However, by MAS beneficial genotypes or genotype combinations can be selected using a single or a few markers, which is suitable for qualitative traits controlled by a single gene locus or less major genes. There are not many qualitative traits in apple and other woody perennials, thus the use of the MAS is far limited. By GS, genotyping is performed using high-density chips containing genome wide SNPs or using next-generation sequencing (NGS), and selection is made with genomic estimated breeding value (GEBV). The GS is suitable for multi-gene quantitative traits. However, the high-density chips and NGS have relatively high costs, and the apple breeding population is often large, such that the GS has been limited either; in addition, the apple has high proportion of non-additive inheritance of important economic traits, and the GS has relatively low prediction accuracy for the non-additive inheritance.

### SUMMARY

The purpose of the present disclosure is to provide a genomics-assisted prediction method for apple fruit quality traits and disease resistance and use thereof.

To realize the above objective, the present disclosure provides the following technical solutions.

The present disclosure provides a set of trait-associated molecular markers in Malus genus, including one or more of molecular markers in Table 1.

TABLE 1
Malus trait-associated molecular markers

| Marker | Associated | | Genomic position | | |
|---|---|---|---|---|---|
| name | trait | Chromosome | (GDDH13 v1.1) | Ref | Alt |
| TSS202 | Soluble solid content | Chr00 | 6,746,071 | C | T |
| TSS203 | Soluble solid content | Chr00 | 12,096,767 | T | C |
| LY064 | Fruit ripening date | Chr00 | 16,403,660 | T | C |
| TSS206 | Soluble solid content | Chr00 | 34,424,368 | A | G |
| SP011 | Fruit shape | Chr01 | 6,583,636 | C | T |
| XDY10 | Fruit shape | Chr01 | 11,115,477 | G | A |
| s7_27 | Fruit fructose content | Chr01 | 22,352,268 | G | T |
| SU203 | Fruit fructose content | Chr01 | 23,318,475 | T | C |
| s8_17 | Fruit fructose content | Chr01 | 23,369,940 | T | C |
| TSS209 | Soluble solid content | Chr01 | 25,049,222 | C | T |
| f2_1 | Fruit fructose content | Chr01 | 25,310,876 | G | T |
| NNENE21 | Fruit fructose content | Chr01 | 25,501,449 | T | C |
| SZ8111 | Fruit weight | Chr01 | 26,192,298 | G | A |
| TSS210 | Soluble solid content | Chr01 | 26,623,907 | A | C |
| LL015-02 | Fruit ripening date | Chr01 | 26,658,863 | T | C |
| SU204 | Fruit fructose content | Chr01 | 27,244,389 | C | G |
| SZ8165 | Fruit weight | Chr01 | 27,290,902 | T | C |
| LY284 | Fruit ripening date | Chr01 | 27,432,954 | A | G |
| MY154 | Fruit ripening date | Chr01 | 27,556,598 | T | A |
| TSS212 | Soluble solid content | Chr01 | 29,044,153 | T | A |
| TSS213-02 | Soluble solid content | Chr01 | 29,200,389 | C | T |
| f4-1 | Fruit fructose content | Chr01 | 29,661,155 | G | A |
| TSS214_ | Soluble solid content | Chr01 | 29,878,772 | C | T |
| TSS215 | Soluble solid content | Chr01 | 31,169,847 | C | T |
| f5_3 | Fruit fructose content | Chr01 | 31,183,130 | G | T |
| TSS216 | Soluble solid content | Chr02 | 2,621,710 | C | T |
| S710 | Fruit ring rot disease | Chr02 | 2,772,109 | A | G |
| | resistance-Lw048 | | | | |
| S733 | Fruit ring rot disease | Chr02 | 6,751,401 | G | A |
| | resistance-Lw048 | | | | |
| wwbb1444 | Fruit storability | Chr02 | 10,479,553 | G | A |
| SZ3463 | Fruit weight | Chr02 | 11,277,610 | G | A |
| wr301 | Fruit storability | Chr02 | 11,357,058 | G | A |
| SZ3571 | Fruit weight | Chr02 | 13,418,996 | A | C |
| TSS217 | Soluble solid content | Chr02 | 14,244,433 | T | C |
| SZ8286 | Fruit weight | Chr02 | 14,633,892 | C | G |
| zwyx71 | Fruit ring rot disease | Chr02 | 14,761,120 | A | T |
| | resistance-Lw048 | | | | |
| XDY32 | Fruit shape | Chr02 | 16,058,673 | G | A |
| ddy6 | Fruit weight | Chr02 | 16,060,933 | G | A |
| SZ3790 | Fruit weight | Chr02 | 20,662,023 | T | C |
| wwbb1063 | Fruit storability | Chr02 | 21,045,042 | G | A |
| SZ8397-02 | Fruit weight | Chr02 | 22,674,062 | A | G |
| SZ12448 | Fruit weight | Chr02 | 23,876,017 | C | A |
| SZ8558 | Fruit weight | Chr02 | 26,929,426 | T | C |
| SZ4161 | Fruit weight | Chr02 | 27,622,440 | A | G |
| f6_25 | Fruit fructose content | Chr03 | 1,341,497 | T | C |
| newdy202 | Fruit shape | Chr03 | 3,150,985 | C | T |
| XDY50 | Fruit shape | Chr03 | 4,516,062 | T | G |
| f8_14 | Fruit fructose content | Chr03 | 4,604,581 | A | T |
| XDY368 | Fruit fructose content | Chr03 | 4,677,137 | A | G |
| zwyx448 | Fruit ring rot disease | Chr03 | 5,372,925 | G | C |
| | resistance-Lw048 | | | | |
| SPO31 | Fruit shape | Chr03 | 6,857,641 | T | C |
| SP032 | Fruit shape | Chr03 | 8,650,170 | A | C |
| NBDY40 | Fruit shape | Chr03 | 10,949,240 | G | T |
| zxxl25 | Fruit ripening date | Chr03 | 12,128,397 | A | G |
| neww378 | Fruit ripening date | Chr03 | 13,366,200 | T | C |
| S391 | Fruit ripening date | Chr03 | 15,156,984 | G | A |
| XLCO42 | Fruit ripening date | Chr03 | 15,359,091 | C | T |
| LL984 | Fruit ripening date | Chr03 | 16,499,163 | C | T |
| WBBBB219 | Fruit storability | Chr03 | 17,338,844 | C | A |
| DDDD2 | Fruit shape | Chr03 | 17,759,874 | G | A |
| CY242 | Fruit ripening date | Chr03 | 17,834,913 | G | A |
| CY564 | Fruit ripening date | Chr03 | 19,114,123 | T | G |
| XDY103 | Fruit shape | Chr03 | 19,585,209 | A | C |
| S395-02 | Fruit ripening date | Chr03 | 22,021,686 | C | T |
| CY603 | Fruit ripening date | Chr03 | 23,418,983 | T | G |
| TSS218 | Soluble solid content | Chr03 | 23,915,298 | G | T |
| GWBI | Fruit ripening date | Chr03 | 25,532,731 | G | C |
| | Fruit storability | | | | |
| CY874 | Fruit ripening date | Chr03 | 26,163,955 | C | G |
| XLC218 | Fruit ripening date | Chr03 | 26,432,199 | C | T |
| WBBB71 | Fruit storability | Chr03 | 26,684,680 | A | T |
| XLC556 | Fruit ripening date | Chr03 | 27,276,582 | A | G |
| nc91 | Fruit storability | Chr03 | 28,907,974 | T | C |
| XWB321 | Fruit ripening date | Chr03 | 29,398,499 | C | A |
| | Fruit storability | | | | |
| LL1499 | Fruit ripening date | Chr03 | 30,229,140 | G | A |
| S372 | Fruit ripening date | Chr03 | 30,500,136 | C | A |
| neww278 | Fruit ripening date | Chr03 | 30,528,992 | T | C |
| GWB636 | Fruit storability | Chr03 | 30,754,593 | C | T |
| LL288 | Fruit ripening date | Chr03 | 31,196,252 | A | G |
| WBBBB13 | Fruit storability | Chr03 | 31,718,792 | G | C |
| LL531 | Fruit ripening date | Chr03 | 31,728,088 | G | A |
| WBBBB85 | Fruit storability | Chr03 | 33,202,419 | T | C |
| LL796 | Fruit ripening date | Chr03 | 33,430,897 | A | G |
| S477 | Fruit ripening date | Chr03 | 34,732,032 | A | G |
| | Fruit storability | | | | |
| MY355 | Fruit ripening date | Chr03 | 35,351,895 | G | T |
| neww45 | Spur tree architecture | Chr03 | 35,480,903 | C | T |
| S484 | Fruit storability | Chr03 | 36,414,853 | G | A |
| TSS219 | Soluble solid content | Chr04 | 6,230,752 | T | C |
| TSS221 | Soluble solid content | Chr04 | 7,779,751 | T | insCTCC |
| TSS222-02 | Soluble solid content | Chr04 | 17,134,357 | A | G |
| TSS223 | Soluble solid content | Chr04 | 18,362,465 | T | C |
| TSS225 | Soluble solid content | Chr04 | 23,107,219 | G | T |
| SP041 | Fruit shape | Chr04 | 23,306,061 | C | T |
| SP043 | Fruit shape | Chr04 | 25,208,568 | C | T |
| SP044 | Fruit shape | Chr04 | 27,497,239 | A | T |
| SP042 | Fruit shape | Chr04 | 30,693,212 | A | T |
| CY1399-02 | Fruit ripening date | Chr05 | 25,051,407 | T | C |
| CY1591 | Fruit ripening date | Chr05 | 28,648,715 | C | T |
| SP051 | Fruit shape | Chr05 | 30,658,901 | G | A |
| zwy6 | Fruit cover color degree | Chr05 | 37,652,411 | A | C |
| color1169 | Fruit cover color degree | Chr05 | 45,823,619 | T | C |
| S1118 | Fruit ring rot disease | Chr06 | 4,827,886 | T | G |
| | resistance-Zz26 | | | | |
| LYY2825 | Fruit ripening date | Chr06 | 10,902,491 | T | C |
| s5_42 | Fruit fructose content | Chr06 | 14,113,242 | G | A |
| SZ8646 | Fruit weight | Chr06 | 32,321,333 | C | T |
| wrl97 | Fruit storability | Chr06 | 32,387,198 | G | A |
| TSS226 | Soluble solid content | Chr07 | 23,239,054 | A | G |
| zwyy29 | Fruit cover color degree | Chr08 | 2,089,437 | G | C |
| colorl31 | Fruit cover color degree | Chr08 | 4,081,610 | C | T |
| TSS228 | Soluble solid content | Chr08 | 4,090,027 | T | C |
| color318 | Fruit cover color degree | Chr08 | 4,870,463 | T | C |
| mb44 | Fruit malate content | Chr08 | 7,674,347 | A | T |
| MA202 | Fruit malate content | Chr08 | 8,728,049 | G | T |
| TSS229 | Soluble solid content | Chr08 | 10,233,508 | A | G |
| s6_75 | Fruit fructose content | Chr08 | 11,373,292 | C | T |
| SAUR-05 | Fruit malate content | Chr08 | 11,648,389 | G | A |
| SAUR-06 | Fruit malate content | Chr08 | 11,648,653 | G | A |
| SP081 | Fruit shape | Chr08 | 13,916,500 | C | T |
| SZ8824 | Fruit weight | Chr09 | 921,113 | G | T |
| TSS232 | Soluble solid content | Chr09 | 18,329,880 | C | A |
| C400 | Fruit cover color | Chr09 | 19,032,403 | A | G |
| | degree | | | | |
| TSS233 | Soluble solid content | Chr09 | 19,435,642 | G | A |
| TSS234 | Soluble solid content | Chr09 | 22,432,228 | G | A |
| TSS235 | Soluble solid content | Chr09 | 24,803,629 | A | C |
| TSS236 | Soluble solid content | Chr09 | 24,807,215 | A | G |
| TSS237 | Soluble solid content | Chr09 | 25,957,418 | A | G |
| TSS238 | Soluble solid content | Chr09 | 27,916,358 | T | C |
| f10_1149 | Fruit fructose content | Chr09 | 30,376,294 | A | G |
| TSS240 | Soluble solid content | Chr09 | 30,893,803 | T | C |
| TSS241 | Soluble solid content | Chr09 | 32,254,297 | A | G |
| TSS242_ | Soluble solid content | Chr09 | 32,808,259 | A | G |
| ZZZ162 | Fruit cover color degree | Chr09 | 35,557,774 | T | A |
| TSS245 | Soluble solid content | Chr09 | 35,814,699 | C | T |
| SU201 | Fruit fructose content | Chr09 | 36,400,519 | T | A |
| color852 | Fruit cover color degree | Chr10 | 1,527,990 | T | C |
| neww26 | Spur tree architecture | Chr10 | 6,721,115 | C | T |
| wwbb1159 | Fruit storability | Chr10 | 9,403,318 | C | T |
| wwbb1277 | Fruit storability | Chr10 | 12,478,767 | T | G |
| wwbb1332 | Fruit storability | Chr10 | 19,778,489 | T | C |
| wwbb1200 | Fruit storability | Chr10 | 25,072,352 | G | C |
| wwbb1373 | Fruit storability | Chr10 | 25,583,532 | A | G |
| S1122 | Fruit ring rot disease | Chr10 | 26,131,644 | T | C |
| | resistance-Zz26 | | | | |
| Zzwyl02 | Fruit ring rot disease | Chr10 | 35,049,752 | G | A |
| | resistance-Zz26 | | | | |
| Hzwy486 | Fruit ring rot disease | Chr10 | 37,766,922 | T | A |
| | resistance-Zz26 | | | | |
| SP101 | Fruit shape | Chr10 | 38,100,894 | A | G |
| wwbb378 | Fruit storability | Chr11 | 1,391,510 | G | A |
| wwbb411 | Fruit storability | Chr11 | 1,922,241 | A | T |
| HB123 | Fruit weight | Chr11 | 3,176,361 | T | C |
| newdy33 | Fruit shape | Chr11 | 4,386,624 | T | A |
| SZ1348 | Fruit weight | Chr11 | 20,016,627 | A | T |
| color1245 | Fruit cover color degree | Chr11 | 29,379,387 | T | C |
| SZ832 | Fruit weight | Chr11 | 29,379,468 | G | C |
| SZ906 | Fruit weight | Chr11 | 32,455,108 | C | T |
| wwbb1441 | Fruit storability | Chr11 | 32,559,107 | T | C |
| Y323 | Fruit ripening date | Chr12 | 3,694,757 | A | C |
| LY591 | Fruit ripening date | Chr12 | 5,361,905 | T | C |
| SZ4250 | Fruit weight | Chr12 | 7,775,358 | A | G |
| Y397 | Fruit ripening date | Chr12 | 7,970,761 | C | T |
| SZ4378 | Fruit weight | Chr12 | 10,871,681 | C | G |
| S1246 | Spur tree architecture | Chr12 | 11,323,438 | C | A |
| zxx51 | Spur tree architecture | Chr12 | 12,132,418 | G | C |
| SZ4457 | Fruit weight | Chr12 | 12,264,258 | C | T |
| SZ4595 | Fruit weight | Chr12 | 15,224,557 | A | C |
| neww5 | Spur tree architecture | Chr12 | 16,395,680 | T | A |
| CY1821 | Fruit ripening date | Chr12 | 16,632,402 | T | A |
| SZ12710 | Fruit weight | Chr12 | 17,104,265 | T | G |
| neww61 | Spur tree architecture | Chr12 | 17,124,574 | T | G |
| wr70 | Fruit storability | Chr12 | 17,767,195 | T | C |
| neww16 | Spur tree architecture | Chr12 | 18,746,403 | G | T |
| SZ9100 | Fruit weight | Chr12 | 21,400,482 | G | T |
| CY2464 | Fruit ripening date | Chr12 | 21,676,040 | C | T |
| wwbb623 | Fruit storability | Chr12 | 22,203,610 | C | T |
| SZ4849 | Fruit weight | Chr12 | 23,710,003 | T | G |
| LY659 | Fruit ripening date | Chr12 | 24,607,594 | C | G |
| wrl54 | Fruit storability | Chr12 | 24,778,590 | A | G |
| wwbb944 | Fruit storability | Chr12 | 28,128,567 | A | C |
| neww19 | Spur tree architecture | Chr12 | 28,321,850 | C | T |
| SZ1413 | Fruit weight | Chr13 | 2,101,864 | C | T |
| SZ4976 | Fruit weight | Chr13 | 3,350,768 | A | C |
| SZ2020 | Fruit weight | Chr13 | 4,926,176 | C | T |
| SZ5112 | Fruit weight | Chr13 | 6,066,422 | C | T |
| SZ9195 | Fruit weight | Chr13 | 6,537,121 | A | G |
| SZ2250 | Fruit weight | Chr13 | 8,952,017 | T | A |
| SZ2270 | Fruit weight | Chr13 | 11,870,499 | G | A |
| SZ2365 | Fruit weight | Chr13 | 13,933,420 | C | G |
| zwyx1188 | Fruit ring rot disease | Chr13 | 19,612,655 | G | C |
| | resistance-Ls1 | | | | |
| Yzwy320 | Fruit ring rot disease | Chr13 | 24,655,606 | G | C |
| | resistance-Lw023 | | | | |
| wwbb1139 | Fruit storability | Chr14 | 3,790,454 | G | A |
| SZ9364 | Fruit weight | Chr14 | 7,682,281 | T | C |
| SZ2614 | Fruit weight | Chr14 | 10,291,436 | C | A |
| TSS247-02 | Soluble solid content | Chr14 | 11,534,642 | C | T |
| TSS248 | Soluble solid content | Chr14 | 12,911,779 | T | G |
| SZ2733 | Fruit weight | Chr14 | 12,985,267 | T | A |
| SZ2805 | Fruit weight | Chr14 | 20,448,307 | A | G |
| SZ2888 | Fruit weight | Chr14 | 23,187,202 | G | T |
| SZ2987 | Fruit weight | Chr14 | 25,545,532 | G | A |
| zwyxl207 | Fruit ring rot disease | Chr14 | 25,700,923 | C | A |
| | resistance-Zz26 | | | | |
| CY1709 | Fruit ripening date | Chr14 | 25,857,739 | G | A |
| SZ3067 | Fruit weight | Chr14 | 27,345,446 | T | A |
| SZ5253 | Fruit weight | Chr14 | 30,020,927 | C | T |
| Wzwy255 | Fruit ring rot disease | Chr14 | 32,273,186 | C | G |
| | resistance-LwO23 | | | | |
| S981 | Fruit ring rot disease | Chr15 | 1,851,440 | G | C |
| | resistance-Lw048 | | | | |
| S1005 | Fruit ring rot disease | Chr15 | 6,167,341 | C | T |
| | resistance-Lsl | | | | |
| SZ9471 | Fruit weight | Chr15 | 6,813,832 | A | G |
| dyy88 | Fruit weight | Chr15 | 6,832,683 | T | C |
| zwyxl080 | Fruit ring rot disease | Chr15 | 7,294,095 | G | C |
| | resistance-Lsl | | | | |
| S1202 | Glomerella leaf blotch | Chr15 | 7,518,836 | G | C |
| | resistance | | | | |
| zhwy64 | Glomerella leaf blotch | Chr15 | 7,535,961 | T | C |
| | resistance | | | | |
| s1_1 | Fruit fructose content | Chr15 | 7,961,469 | C | T |
| XDY359 | Fruit fructose content | Chr15 | 8,149,862 | A | T |
| HB15 | Fruit fructose content | Chr15 | 11,982,626 | A | G |
| SZ1609 | Fruit weight | Chr15 | 13,283,951 | G | A |
| XDY354 | Fruit fructose content | Chr15 | 14,556,321 | T | C |
| wwl9 | Spur tree architecture | Chr15 | 14,791,080 | A | G |
| ddyl9 | Fruit fructose content | Chr15 | 15,628,743 | C | T |
| TSS258-04 | Soluble solid content | Chr15 | 15,630,626 | G | A |
| TSS249 | Soluble solid content | Chr15 | 15,632,755 | A | G |
| TSS258-09 | Soluble solid content | Chr15 | 15,633,545 | T | C |
| s2_6 | Fruit fructose content | Chr15 | 16,053,147 | T | A |
| TSS250 | Soluble solid content | Chr15 | 16,541,855 | C | G |
| S313 | Fruit ripening date | Chr15 | 16,567,767 | G | A |
| SZ5388 | Fruit weight | Chr15 | 18,184,482 | A | C |
| S1252 | Spur tree architecture | Chr15 | 18,281,416 | C | A |
| XDY127 | Fruit weight | Chr15 | 18,810,707 | A | G |
| SZ9751 | Fruit weight | Chr15 | 20,670,273 | C | A |
| wwbb112 | Fruit storability | Chr15 | 20,994,595 | C | T |
| SZ5464 | Fruit weight | Chr15 | 22,313,813 | A | T |
| S321 | Fruit ripening date | Chr15 | 24,229,527 | A | G |
| XDY157 | Fruit weight | Chr15 | 24,269,269 | T | G |
| TSS251-02 | Soluble solid content | Chr15 | 24,616,358 | T | C |
| zwyx925 | Fruit ring rot disease | Chr15 | 25,553,244 | A | G |
| | resistance-Lw048 | | | | |
| LYY094 | Fruit ripening date | Chr15 | 25,641,444 | T | C |
| zwyx555 | Fruit ring rot disease | Chr15 | 25,773,261 | A | C |
| | resistance-Lw048 | | | | |
| XDY160 | Fruit weight | Chr15 | 26,425,887 | G | A |
| wwbb20 | Fruit storability | Chr15 | 26,869,888 | G | A |
| TSS252 | Soluble solid content | Chr15 | 26,963,022 | G | A |
| TSS253 | Soluble solid content | Chr15 | 26,994,265 | G | A |
| LYY347 | Fruit ripening date | Chr15 | 27,007,585 | G | C |
| SZ5769 | Fruit weight | Chr15 | 27,186,324 | A | G |
| TSS254 | Soluble solid content | Chr15 | 30,285,932 | G | T |
| neww441 | Fruit ripening date | Chr15 | 30,869,138 | G | A |
| colorl450 | Fruit cover color degree | Chr15 | 31,183,115 | T | C |
| SZ5929 | Fruit weight | Chr15 | 31,402,325 | C | T |
| TSS255 | Soluble solid content | Chr15 | 31,521,236 | C | T |
| wwbb181 | Fruit storability | Chr15 | 31,983,275 | G | A |
| SZ6076 | Fruit weight | Chr15 | 33,640,614 | C | A |
| neww146 | Fruit ripening date | Chr15 | 34,629,451 | A | G |
| SZ273 | Fruit weight | Chr15 | 37,109,478 | A | C |
| XDY231 | Fruit shape | Chr15 | 37,110,278 | A | G |
| SZ6268 | Fruit weight | Chr15 | 38,111,629 | G | T |
| S349 | Fruit ripening date | Chr15 | 38,480,628 | T | C |
| xccc60 | Fruit ripening date | Chr15 | 40,445,622 | A | G |
| SZ10554 | Fruit weight | Chr15 | 40,559,644 | G | A |
| TSS260 | Soluble solid content | Chr15 | 40,795,162 | C | A |
| TSS261 | Soluble solid content | Chr15 | 41,354,666 | G | C |
| SZ6459 | Fruit weight | Chr15 | 42,126,761 | G | A |
| SZ10597 | Fruit weight | Chr15 | 44,293,907 | T | C |
| TSS263 | Soluble solid content | Chr15 | 44,777,216 | C | A |
| XDY302 | Fruit shape | Chr15 | 44,788,654 | A | C |
| SZ6675 | Fruit weight | Chr15 | 46,848,583 | C | T |
| wwbb244 | Fruit storability | Chr15 | 47,522,379 | T | C |
| BDY17 | Fruit shape | Chr15 | 47,794,257 | T | G |
| SZ6815 | Fruit weight | Chr15 | 48,048,672 | G | C |
| SZ6840 | Fruit weight | Chr15 | 50,117,476 | A | G |
| NCVXX77 | Fruit shape | Chr15 | 50,136,492 | C | T |
| TSS264 | Soluble solid content | Chr15 | 50,153,850 | T | C |
| SZ7016 | Fruit weight | Chr15 | 51,632,414 | A | G |
| SZ7070 | Fruit weight | Chr15 | 53,318,871 | A | C |
| XDY315 | Fruit shape | Chr15 | 53,446,034 | G | A |
| S1234 | Spur tree architecture | Chr16 | 509,607 | A | T |
| SZ11309 | Fruit weight | Chr16 | 574,182 | T | A |
| GWB581 | Fruit storability | Chr16 | 1,440,735 | T | C |
| XWB137 | Fruit storability | Chr16 | 1,540,780 | G | A |
| XWB392 | Fruit storability | Chr16 | 3,059,129 | T | C |
| Ma | Fruit malate content | Chr16 | 3,179,027 | G | A |
| SZ3450 | Fruit weight | Chr16 | 3,299,638 | T | A |
| S616 | Fruit procyanidin | Chr16 | 3,404,957 | C | G |
| | B2 content | | | | |
| UDY1 | Soluble solid content | Chr16 | 3,441,632 | G | A |
| TSS266 | Soluble solid content | Chr16 | 3,452,478 | A | C |
| FDY190 | Fruit weight | Chr16 | 3,552,434 | A | T |
| TSS267 | Soluble solid content | Chr16 | 3,554,158 | G | A |
| S641 | Fruit procyanidin | Chr16 | 3,804,010 | T | C |
| | B2 content | | | | |
| S652 | Fruit procyanidin | Chr16 | 3,887,901 | T | G |
| | B2 content | | | | |
| TSS268 | Soluble solid content | Chr16 | 4,273,438 | C | A |
| DYS17 | Soluble solid content | Chr16 | 5,309,087 | A | G |
| TSS269 | Soluble solid content | Chr16 | 5,583,186 | T | C |
| S504 | Fruit storability | Chr16 | 5,979,221 | C | T |
| WBBBB132 | Fruit storability | Chr16 | 5,979,292 | T | A |
| XDY346 | Fruit weight | Chr16 | 6,334,724 | C | T |
| newdy292 | Fruit weight | Chr16 | 6,398,049 | T | C |
| XWB291 | Fruit storability | Chr16 | 9,127,269 | T | C |
| SZ3158 | Fruit weight | Chr16 | 9,187,936 | A | G |
| Y108 | Fruit ripening date | Chr16 | 9,422,810 | C | A |
| SZ7418 | Fruit weight | Chr16 | 9,428,581 | A | G |
| TSS271 | Soluble solid content | Chr16 | 9,470,752 | T | A |
| XWB111 | Fruit storability | Chr16 | 10,070,698 | A | G |
| SZ7516 | Fruit weight | Chr16 | 10,928,020 | T | G |
| S402 | Fruit storability | Chr16 | 13,200,599 | A | T |
| SZ7624 | Fruit weight | Chr16 | 13,203,946 | A | G |
| LY770 | Fruit ripening date | Chr16 | 13,243,241 | C | G |
| S412 | Fruit storability | Chr16 | 13,579,418 | A | C |
| SZ11912 | Fruit weight | Chr16 | 13,933,577 | G | A |
| GWB262-02 | Fruit storability | Chr16 | 14,398,932 | G | A |
| LYY3327 | Fruit ripening date | Chr16 | 14,729,859 | A | T |
| S358 | Fruit ripening date | Chr16 | 16,542,947 | C | A |
| S369 | Fruit ripening date | Chr16 | 18,318,696 | T | G |
| LC06 | Fruit ripening date | Chr16 | 20,598,023 | A | C |
| S432 | Fruit storability | Chr16 | 20,996,010 | A | C |
| XL13 | Fruit ripening date | Chr16 | 22,704,725 | T | C |
| S407 | Fruit storability | Chr16 | 23,366,479 | C | T |
| nc22 | Fruit storability | Chr16 | 38,573,461 | T | A |
| S468 | Fruit storability | Chr16 | 38,573,599 | C | A |
| TSS272_ | Soluble solid content | Chr16 | 38,914,076 | T | C |
| TSS274 | Soluble solid content | Chr17 | 5,996,233 | T | C |
| neww27 | Spur tree architecture | Chr17 | 7,843,681 | G | T |
| neww28 | Spur tree architecture | Chr17 | 9,275,707 | T | C |
| CHL1 | Fruit chlorogenate | Chr17 | 15,953,079 | TACT | del |
| | content | | | GCCC | |
| | | | | AA | |
| wwbb289 | Fruit storability | Chr17 | 21,947,295 | G | T |
| LY836 | Fruit ripening date | Chr17 | 22,524,548 | T | G |
| CA201 | Fruit chlorogenate | Chr17 | 23,199,602 | A | G |
| | content | | | | |
| S602 | Fruit chlorogenate | Chr17 | 27,353,986 | G | A |
| | content | | | | |

The present disclosure further provides a genotype effect value or a genotype combination effect value of each genotype of the molecular markers on a corresponding trait as shown in Table 2.

TABLE 2
Genotype effect value of each maker genotype or genotype combination on the
corresponding trait

| (1) Fruit malate content or juice pH: | | | | |
|---|---|---|---|---|
| Marker | pH | Malate | | |
| Ma | | | | |
| AA | 0.45 | −3.09 | | |
| GA | −0.06 | 0.18 | | |
| GG | −0.18 | 1.12 | | |
| MA202 | | | | |
| GG | 0.06 | −0.11 | | |
| GT | 0.03 | −1.16 | | |
| TT | −0.1 | 1.82 | | |
| mb44 | | | | |
| AA | 0.14 | −2.14 | | |
| A/insT | −0.84 | 1.22 | | |
| AT | 0.04 | −0.69 | | |
| insT | −0.94 | | | |
| TT | −0.07 | 2.12 | | |
| SAUR-05 | | | | |
| AA | −0.14 | 1.36 | | |
| GA | −0.01 | 0.07 | | |
| GG | 0.02 | −0.5 | | |
| SAUR-06 | | | | |
| AA | −0.09 | 1.78 | | |
| GA | 0 | −0.06 | | |
| GG | 0.03 | −0.52 | | |
| Genotype combination effect of Ma/MA202/SAUR-5 on fruit malate content | | | | |
| Ma | MA202 | SAUR-5 | | |
| AA | GG | GA | −3.25 | |
| AA | GG | GG | −4.44 | |
| AA | GT | AA | −1.54 | |
| AA | GT | GA | −3.5 | |
| AA | GT | GG | −2.87 | |
| AA | TT | AA | −3.09 | |
| AA | TT | GA | −2.55 | |
| AA | TT | GG | −3.09 | |
| GA | GG | AA | | |
| GA | GG | GA | 0.11 | |
| GA | GG | GG | 0.72 | |
| GA | GT | AA | | |
| GA | GT | GA | −0.72 | |
| GA | GT | GG | −1.85 | |
| GA | TT | AA | 1.96 | |
| GA | TT | GA | 2.01 | |
| GA | TT | GG | 1.53 | |
| GG | GG | AA | | |
| GG | GG | GA | 1.86 | |
| GG | GG | GG | 0.33 | |
| GG | GT | AA | | |
| GG | GT | GA | −0.38 | |
| GG | GT | GG | 3.32 | |
| GG | TT | AA | 5.97 | |
| GG | TT | GA | 2.36 | |
| GG | TT | GG | 1 | |
| (2) Genotype combination effect of fruit chlorogenate content: | | | | |
| CA201 | S602 | CHL1 | GPV | |
| AA | AG | ins/del | 324 | |
| AA | AG | ins | 363.93 | |
| AA | GG | ins/del | 914.31 | |
| AA | GG | ins | 1041.67 | |
| AG | AG | ins/del | 467.05 | |
| AG | AG | ins | 521.6 | |
| AG | GG | ins/del | 535.2 | |
| AG | GG | ins | 855.94 | |
| (3) Genotype combination effect of fruit procyanidin B2 content: | | | | |
| S616 | S641 | S652 | GPV | |
| CC | TT | GT | 149.74 | |
| CC | TT | TT | 179.91 | |
| GC | CC | GG | 133.4 | |
| GC | CT | GG | 365.82 | |
| GC | CT | GT | 392.45 | |
| GC | CT | TT | 237.36 | |
| GC | TT | GT | 62.01 | |
| GC | TT | TT | 367.72 | |
| GG | CT | GT | 1155.12 | |
| (4) Fruit cover color degree: | | | | |
| C400 | | C1169 | | |
| AA | 1.11 | CC | 5.07 | |
| AG | −0.45 | TC | −3.48 | |
| GG | 21.65 | TT | 1.34 | |
| C1245 | | C131 | | |
| CC | 0.5 | CC | −0.46 | |
| TC | 0.42 | CT | 1.03 | |
| TT | −4.9 | | | |
| C1450 | | C318 | | |
| CC | −2.93 | CC | 43.65 | |
| TA | 3.65 | TC | 1.03 | |
| TC | 0.84 | TT | −0.53 | |
| TT | −0.01 | | | |
| C852 | | Zwy6 | | |
| TC | 1.18 | AA | 1.31 | |
| TT | −0.55 | AC | −2.58 | |
| CC | −17.88 | | | |
| Zwy29 | | Z162 | | |
| CC | 33.65 | AA | 13.11 | |
| GC | 0.4 | TA | 4.08 | |
| GG | −0.23 | TT | −16.63 | |
| Genotype combination effect of fruit cover color degree: | | | | |
| Z162 | Zwy6 | | C1245 | |
| AA | AA | 13.47 | CC | 10.53 |
| AA | AC | 12.65 | TC | 18.72 |
| AA | CC | −13.85 | TT | 2.4 |
| TA | AA | 4.65 | CC | 2.93 |
| TA | AC | 3.69 | TC | 4.98 |
| TA | CC | −15.34 | TT | 5.11 |
| TT | AA | −14.66 | CC | −17.93 |
| TT | AC | −20.82 | TC | −16.04 |
| TT | CC | −51.35 | TT | −15.76 |

(5) Fruit ring rot disease resistance:

| S1005 | | Z1080 | | Z1188 | |
|---|---|---|---|---|---|
| CC | −1.74 | CC | −5.75 | GC | −0.25 |
| CT | −1.27 | GC | −0.59 | GG | 0.11 |
| TT | 2.19 | GG | 1.11 | | |
| W2255 | | Y2320 | | S710 | |
| CC | 1.12 | GC | −0.34 | AA | 0.54 |
| CG | −1.02 | GG | 0.23 | AG | 1.47 |
| GG | 0.54 | GG | −2.33 | | |
| S733 | | S981 | | Zwy448 | |
| AA | −3.19 | CC | 3.28 | CC | −2.39 |
| GA | 2.04 | GC | 1.74 | GC | 0.72 |
| GG | −0.52 | GG | −1.05 | GG | 12.9 |
| Zwy555 | | Zwy71 | | Zwy925 | |
| AA | 0.36 | AA | 0.83 | AA | 0.32 |
| AC | −0.25 | AT | 0.51 | AG | −1.03 |
| CC | −1.91 | TT | −2.42 | GG | 6.66 |
| Hzwy486 | | S1118 | | S1122 | |
| AA | −1.02 | GG | −0.13 | CC | −1.83 |
| TA | 0.09 | TG | −0.75 | TC | 0.14 |
| TT | 0.18 | TT | 1.02 | TT | −0.15 |
| Zwy1207 | | Zwy102 | | | |
| AA | −2.61 | AA | −17.65 | | |
| CA | 2.69 | GA | 1.11 | | |
| CC | 0.75 | GG | −0.41 | | |

(6) Fruit ripening date:

| C1399 | | C1591 | | C1709 | |
|---|---|---|---|---|---|
| CC | 2.74 | CC | −3.55 | AA | −9.92 |
| TC | 2.45 | CT | 2.5 | GA | 1.27 |
| TT | −9.33 | TT | 6.65 | GG | 1.59 |
| C1821 | | C242 | | C2464 | |
| AA | 9.81 | AA | −16.45 | CC | 1.85 |
| TA | −1.19 | GA | 8.22 | CT | −0.79 |
| TT | −1.6 | GG | −5.44 | TT | 0.53 |
| C564 | | C603 | | C874 | |
| GG | 1.27 | TG | 9.8 | CC | −6.41 |
| TG | −1.35 | TT | −5.96 | CG | 10.81 |
| TT | 2.45 | | | | |
| LC06 | | LL015 | | LL1149 | |
| AA | −10.45 | CC | 1 | AA | −11.45 |
| AC | 0.82 | TC | 0.62 | GA | 9.24 |
| CC | 2.21 | TT | −1.66 | GG | −5.88 |
| LL288 | | LL531 | | LL796 | |
| AA | −9.99 | AA | 1.38 | AA | −38.83 |
| AG | −1.44 | GA | −0.71 | AG | −2.69 |
| GG | 3.14 | GG | −16.49 | GG | 4.89 |
| LL984 | | LY064 | | LY284 | |
| CC | −5.51 | CC | 5.54 | AA | 1.47 |
| CT | 8.53 | TC | −0.23 | AG | 0.45 |
| TT | −23.12 | TT | −0.58 | GG | −23.04 |
| LY591 | | LY659 | | LY770 | |
| CC | 1.44 | CC | 5.31 | CC | 0.76 |
| TC | 0.27 | CG | 0.88 | CG | −0.31 |
| TT | −2.23 | GG | −1.6 | GG | −22.57 |
| LY836 | | LYY094 | | LYY2825 | |
| CC | −46.45 | CC | −0.34 | CC | 1.61 |
| GG | −46.45 | TC | 1.57 | TC | −3.63 |
| TG | 0.86 | TT | −0.88 | | |
| TT | −0.51 | | | | |
| LYY3327 | | LYY347 | | MY154 | |
| AA | −4.44 | CC | 7.06 | AA | 4.4 |
| AT | 2.56 | GC | 2.07 | TA | 0.64 |
| TT | −7.84 | GG | −3.82 | TT | −1.05 |
| MY355 | | new146 | | new278 | |
| GG | −11.92 | AA | −0.56 | CC | −11.45 |
| GT | −1.24 | AG | −0.52 | TC | 15.55 |
| TT | 2.96 | GG | 1.22 | TT | −4.89 |
| new378 | | new441 | | S313 | |
| CC | −16.45 | AA | −10.37 | AA | −7.36 |
| TC | 8.26 | GA | −0.23 | GA | −0.59 |
| TT | −5.34 | GG | 6.2 | GG | 0.67 |
| S321 | | S349 | | | |
| AA | −0.24 | CC | −17.54 | | |
| AG | 0.79 | TC | −1.1 | | |
| GG | −5.23 | TT | 5.48 | | |
| S369 | | S358 | | S372 | |
| CC | −9.62 | AA | 4.13 | AA | 4.69 |
| CG | 9.48 | AG | −38.69 | CA | 0.16 |
| GG | −3.98 | CA | 1.34 | CC | −11.3 |
| TC | 2.7 | CC | −10.12 | | |
| TG | −1.29 | CG | −25.9 | | |
| TT | −11.97 | GG | −43.85 | | |
| S391 | | S395 | | xcc60 | |
| AA | −16.45 | CC | −0.19 | AA | 5.91 |
| AG | 8.1 | CT | −0.81 | AG | −0.51 |
| GG | −5.31 | TT | 3.47 | GG | −20.88 |
| XL13 | | XLC042 | | XLC218 | |
| CC | −10.27 | CC | −3.38 | CC | −15.15 |
| TC | 2.68 | CT | 10.37 | CT | −1.13 |
| TT | −2.45 | TT | −6.45 | TT | 11.52 |
| XLC556 | | Y108 | | Y323 | |
| AA | −14.86 | AA | −2.87 | AA | 21.05 |
| AG | −1.42 | CA | 2.51 | AC | −4.39 |
| GG | 11.1 | CC | −2.36 | CC | 1.6 |
| Y397 | | ZXX125 | | GWB1 | |
| CC | −0.22 | AA | −5.24 | GC | 14.74 |
| CT | 1.44 | AG | 8.08 | GG | −4.37 |
| TT | −2.62 | GG | −16.45 | | |
| S477 | | XWB321 | | | |
| AA | −14.65 | AA | −16.52 | | |
| AG | −1.53 | CA | −0.58 | | |
| GG | 4.22 | CC | 0.65 | | |

(7) Fruit weight:
a. Fixed effect

| | | |
|---|---|---|
| XDY160 | AA | −104.8 |
| S4849 | GG | −100.7 |
| S4161 | GG | −101.4 |

b. Non−fixed effect

| BDY17 | | DDDD2 | | NBDY40 | |
|---|---|---|---|---|---|
| GG | 31.55 | AA | −31.7 | GG | −5.81 |
| TG | 17.13 | GA | −10.3 | GT | 7.12 |
| TT | −8.12 | GG | 8.86 | TT | −8.18 |
| newdy202 | | newdy33 | | SP011 | |
| CC | 16.69 | AA | −9.86 | CC | −5.11 |
| CT | −33.16 | TA | 0.85 | CT | 5.21 |
| TT | −102.68 | TT | 3.42 | TT | −6.59 |
| SP031 | | SP032 | | SP041 | |
| CC | −9.85 | AA | 18.06 | CC | 1.96 |
| TC | 4.85 | AC | −8.65 | CT | −1 |
| TT | 38.59 | CC | −14.94 | TT | −11.92 |
| SP042 | | SP043 | | SP044 | |
| AA | 11.03 | CC | 0.45 | AA | 13.91 |
| AT | −1.69 | CT | 3 | AT | 0.1 |
| TT | −37.88 | TT | −22.43 | TT | −7.2 |
| SP051 | | SP081 | | SP101 | |
| AA | 42.27 | AT | −94.56 | AA | 12.78 |
| GA | 6.78 | CA | −70.99 | AG | 0.59 |
| GG | −8.4 | CC | −19.98 | GG | −36.68 |
| CT | 19.3 | | | | |
| TT | −7.96 | | | | |
| XDY10 | | XDY103 | | XDY231 | |
| AA | 51.74 | AA | 61.78 | AA | 24.53 |
| AC | −101.63 | AC | 7.38 | AG | 17.27 |
| GA | −6.23 | CC | −3.14 | GG | −10.77 |
| GC | −96.44 | | | | |
| GG | −11.74 | | | | |
| XDY302 | | XDY315 | | XDY32 | |
| AA | 2.45 | AA | −58.28 | AA | −11.57 |
| AC | 5.96 | GA | 6.98 | GA | −13.36 |
| CC | −6.2 | GG | −2.13 | GG | 54.74 |
| XDY50 | | ddy6 | | dyy88 | |
| GG | −4.44 | AA | −103.2 | CC | 29.49 |
| TG | 3.89 | GA | −40.37 | TC | 37.89 |
| TT | −3.16 | GG | 18.37 | TT | −5.91 |
| FDY190 | | HB123 | | newdy292 | |
| AA | 15.23 | CC | 5.45 | CC | −104.13 |
| AT | −36.88 | TA | −103.18 | TC | −37.93 |
| TC | −9.37 | TT | 17.03 | | |
| TT | 6.96 | | | | |
| S10554 | | S10597 | | S11309 | |
| AA | −102.64 | CC | −102.64 | AA | −104.59 |
| GA | −39.72 | TC | −39.42 | TA | −36.68 |
| GG | 20.67 | TG | −103.63 | TT | 15.55 |
| TT | 20.64 | | | | |
| S11912 | | S12448 | | S12710 | |
| AA | −86.63 | AA | −102.8 | GG | −59.7 |
| GA | −37.79 | CA | −39.7 | TG | −35.66 |
| GG | 15.59 | CC | 18.18 | TT | 15.48 |
| S1348 | | S1413 | | S1609 | |
| AA | −6.15 | CC | −9.58 | AA | 0.97 |
| AT | 7.49 | CT | 13.62 | GA | −3.62 |
| TT | 29.47 | TT | 6.61 | GG | 3.72 |
| S2020 | | S2250 | | S2270 | |
| CC | 14.82 | AA | −28.59 | AA | −104.23 |
| CT | −40.42 | TA | −2.53 | GA | −38.14 |
| TT | −101.77 | TT | 2.61 | GG | 13.62 |
| S2365 | | S2614 | | S273 | |
| CC | 12.46 | AA | −80.18 | AA | 17.03 |
| CG | −36.54 | CA | −33.46 | AC | −36.99 |
| GG | −104.22 | CC | 21.78 | CC | −104.31 |
| S2733 | | S2805 | | S2888 | |
| AA | −96.57 | AA | 21.63 | GG | 16.51 |
| TA | −38.26 | AG | −38.28 | GT | −15.79 |
| TT | 18.93 | GG | −84.4 | TT | −61.16 |
| S2987 | | S3067 | | S3158 | |
| GA | −24.13 | AA | −82.09 | AA | 16.74 |
| GG | 7.91 | TA | −39.61 | AG | −37.57 |
| TT | 21.58 | GG | −104.13 | | |
| S3450 | | S3463 | | S3571 | |
| AA | −13.46 | AA | −61.89 | AA | 15.89 |
| TA | 7.66 | GA | −23.28 | AC | −37.41 |
| TT | −9.77 | GG | 9.13 | CC | −40.29 |
| S3790 | | S4161 | | S4250 | |
| CC | −102.1 | AA | 18.66 | AA | 16.02 |
| TC | −39.31 | AG | −39.18 | AG | −30.38 |
| TT | 17.47 | GG | −96.71 | GG | −51.89 |
| S4378 | | S4457 | | S4595 | |
| CC | 19.81 | CC | 19.04 | AA | 15 |
| CG | −34.4 | CT | −34.9 | AC | −11.62 |
| GG | −78.84 | TT | −70.14 | CC | −70.86 |
| CT | −88.3 | | | | |
| S4849 | | S4976 | | S5112 | |
| GG | −101.95 | AA | 14.86 | CC | 13.74 |
| TG | −41.21 | AC | −39.84 | CT | −39.1 |
| TT | 18.24 | CC | −99.67 | TT | −101.14 |
| S5253 | | S5388 | | S5464 | |
| CC | 20.05 | AA | 16.92 | AA | 21.12 |
| CT | −38.2 | AC | −36.69 | AT | −40.13 |
| TT | −97.5 | CC | −90.88 | TT | −99.31 |
| S5769 | | S5929 | | S6076 | |
| AA | 19.67 | CA | −100.45 | AA | −103.05 |
| AG | −39.38 | CC | 18.88 | CA | −40.28 |
| GG | −102.88 | CT | −38.24 | CC | 20.95 |
| TT | −102.88 | | | | |
| S6268 | | S6459 | | S6675 | |
| AT | −104.92 | AA | −88.2 | CC | 17.87 |
| GA | −103.63 | AG | −39.69 | CT | −36.9 |
| GG | 20.19 | GG | 20.51 | TT | −102.97 |
| GT | −39.65 | | | | |
| TT | −101.87 | | | | |
| S6815 | | S6840 | | S7016 | |
| CC | −103.05 | AA | 18.25 | AA | 19.12 |
| GC | −38.89 | AG | −37.43 | AG | −37.91 |
| GG | 20.16 | GG | −105.25 | GG | −105.57 |
| S7070 | | S7418 | | S7516 | |
| AA | 17.49 | AA | 16.08 | TG | −33.66 |
| AC | −36.86 | AG | −36.83 | TT | 13.28 |
| CC | −83.3 | | | | |
| S7624 | | S8111 | | S8165 | |
| AA | 1.35 | AA | −63.34 | CC | 38.37 |
| AG | −38.89 | GA | −8.31 | TC | 31.1 |
| GG | 6.65 | TT | −4.96 | | |
| S8286 | | S832 | | S8397 | |
| CC | 14.37 | CC | −28.4 | AA | −6.42 |
| CG | −37.03 | GC | 3.28 | AG | −5.81 |
| GG | 11.54 | GG | 13.76 | | |
| S8558 | | S8646 | | S8824 | |
| CC | 5.74 | CC | −48.12 | GA | −59.11 |
| TC | −4.98 | CT | 2.58 | GG | −17.84 |
| TT | −22.42 | TT | −0.16 | GT | 21.36 |
| TT | 29.89 | | | | |
| S906 | | S9100 | | S9195 | |
| CC | 21.17 | GG | 18.22 | AA | 15.1 |
| CT | 9.25 | GT | −40.89 | AG | −40.43 |
| TT | −27.6 | TT | −101.78 | GG | −102.53 |
| S9364 | | S9471 | | S9751 | |
| CC | −82.43 | AA | −5.91 | AA | 4.08 |
| TC | −35.83 | AG | 35.09 | CA | 0.25 |
| TT | 20.18 | GG | 10.76 | CC | −7.17 |
| XDY127 | | XDY157 | | XDY160 | |
| AA | −4.34 | GG | −100 | AA | −104.46 |
| AG | 31.12 | TG | −40.69 | GA | −39.4 |
| TT | 21.67 | GG | 20.13 | | |
| XDY346 | | NCVX7 | | | |
| CC | 10.22 | CC | −8.19 | | |
| CT | −27.32 | CT | 0.05 | | |
| TT | 55.47 | | | | |

(8) Fruit storability, including four traits: flesh firmness at harvest, flesh crispness at harvest, flesh firmness retainability,
and flesh crispness retainability:

| | flesh firmness at | flesh crispness at | flesh firmness | flesh crispness |
|---|---|---|---|---|
| Marker | harvest | harvest | retainability | retainability |
| GWB1 | | | | |
| GC | 1.7 | 0.14 | 1.01 | 0.8 |
| GG | −0.52 | −0.04 | −0.35 | −0.28 |
| S477 | | | | |
| AA | −1.3 | 0.02 | −0.82 | −0.58 |
| AG | −0.33 | −0.01 | −0.31 | −0.25 |
| GG | 0.56 | 0.01 | 0.39 | 0.31 |
| XWB321 | | | | |
| AA | −0.49 | 0.12 | | |
| CA | −0.42 | −0.02 | −0.28 | −0.22 |
| CC | 0.32 | 0.02 | 0.19 | 0.16 |
| GWB262 | | | | |
| AA | 0.34 | 0.04 | 0.33 | 0.27 |
| delG | −4.98 | −0.51 | 1.59 | 1.81 |
| G/delG | −2 | −0.27 | −1.74 | −1.52 |
| GA | −0.05 | −0.01 | −0.15 | |
| GG | −1.19 | −0.08 | −0.65 | |
| GWB581 | | | | |
| CC | | | | |
| TC | 0.99 | 0.09 | 0.44 | 0.3 |
| TT | −0.45 | −0.04 | −0.27 | −0.17 |
| GWB636 | | | | |
| CC | −0.33 | −0.01 | −0.32 | −0.25 |
| CT | 0.56 | 0.01 | 0.47 | 0.36 |
| TT | −0.64 | 0.19 | 0.59 | 0.81 |
| nc22 | | | | |
| AA | 1.13 | 0.16 | 0.82 | 0.77 |
| TA | 0.07 | 0.01 | 0.04 | 0 |
| TT | −0.45 | −0.06 | −0.31 | −0.24 |
| nc91 | | | | |
| CC | 0.35 | 0 | 0.32 | 0.24 |
| TC | −0.31 | −0.01 | −0.21 | −0.14 |
| TT | 0.18 | 0.09 | −0.24 | −0.25 |
| S402 | | | | |
| A/delA | 3.56 | 0.35 | | |
| AA | −1.14 | −0.14 | −0.8 | −0.64 |
| AT | −0.1 | 0 | −0.12 | −0.1 |
| delA | | | | |
| TT | 0.51 | 0.05 | 0.43 | 0.36 |
| S407 | | | | |
| CC | −0.88 | −0.07 | −0.54 | −0.41 |
| CT | 0.61 | 0.04 | 0.43 | 0.29 |
| TT | 1.66 | 0.18 | 1 | 0.9 |
| S412 | | | | |
| AA | −1.25 | −0.11 | −0.81 | −0.63 |
| AC | −0.08 | −0.01 | −0.13 | −0.11 |
| CC | 0.56 | 0.06 | 0.46 | 0.38 |
| S432 | | | | |
| AA | −1.09 | −0.07 | −0.72 | −0.58 |
| AC | −0.23 | −0.01 | −0.14 | −0.11 |
| CC | 0.68 | 0.04 | 0.43 | 0.35 |
| S468 | | | | |
| AA | 1.3 | 0.1 | 0.79 | 0.55 |
| CA | −0.45 | −0.04 | −0.31 | −0.29 |
| CC | −0.18 | −0.01 | −0.11 | −0.01 |
| S484 | | | | |
| AA | 0.28 | 0.27 | 1.59 | 2.81 |
| GA | −0.38 | −0.03 | −0.33 | −0.25 |
| GG | 0.3 | 0.02 | 0.24 | 0.18 |
| S504 | | | | |
| CC | −0.59 | −0.04 | −0.35 | −0.28 |
| CT | 1.14 | 0.09 | 0.59 | 0.48 |
| TT | 3.5 | −0.39 | | |
| WBB71 | | | | |
| AA | 0.81 | 0.04 | 0.3 | 0.23 |
| AT | −0.85 | −0.06 | −0.37 | −0.27 |
| TT | −0.61 | 0.18 | −0.78 | −0.67 |
| WBB13 | | | | |
| GC | 1.69 | 0.12 | 1.14 | 0.86 |
| GG | −0.58 | −0.04 | −0.43 | −0.32 |
| WBB132 | | | | |
| AA | 3.5 | −0.39 | | |
| TA | 1.14 | 0.09 | 0.59 | 0.48 |
| TT | −0.59 | −0.04 | −0.35 | −0.28 |
| WBB219 | | | | |
| CA | 1.57 | 0.13 | 0.84 | 0.71 |
| CC | −0.47 | −0.04 | −0.28 | −0.23 |
| WBB85 | | | | |
| CC | 0.31 | −0.01 | 0.24 | 0.18 |
| TC | −0.87 | 0 | −0.65 | −0.49 |
| TT | 0.44 | 0.05 | 0.14 | 0.15 |
| wr154 | | | | |
| AA | −0.6 | −0.05 | −0.22 | −0.22 |
| AG | 1.26 | 0.11 | 0.41 | 0.41 |
| GG | 1.28 | 0.1 | | |
| wr197 | | | | |
| AA | 1.17 | 0.01 | | |
| GA | 0.77 | 0.05 | 0.31 | 0.25 |
| GG | −0.38 | −0.02 | −0.18 | −0.14 |
| wr301 | | | | |
| AA | −1.03 | 0.31 | | |
| GA | 1.09 | 0.07 | 0.79 | 0.52 |
| GG | −0.35 | −0.02 | −0.24 | −0.15 |
| wr70 | | | | |
| CC | −1.81 | −0.19 | | |
| TC | 0.97 | 0.09 | 0.35 | 0.38 |
| TT | −0.49 | −0.04 | −0.2 | −0.21 |
| wwbb1063 | | | | |
| AA | 1.09 | 0.13 | 0.88 | 0.55 |
| GA | −0.42 | −0.04 | −0.35 | −0.26 |
| GG | −0.16 | −0.02 | −0.12 | −0.02 |
| wwbb112 | | | | |
| cc | −0.56 | −0.04 | −0.28 | −0.2 |
| CT | −0.01 | −0.02 | 0.05 | 0.03 |
| TT | 0.43 | 0.06 | 0.13 | 0.13 |
| wwbb1139 | | | | |
| AA | −1.51 | −0.15 | −0.68 | −0.45 |
| GA | −0.1 | 0.02 | −0.07 | −0.05 |
| GG | 0.88 | 0.05 | 0.35 | 0.25 |
| wwbb1159 | | | | |
| CC | −0.14 | 0 | −0.25 | −0.17 |
| CT | −0.15 | −0.01 | −0.08 | −0.02 |
| TT | 0.95 | 0.05 | 0.93 | 0.53 |
| wwbb1200 | | | | |
| CC | −0.41 | −0.02 | −0.15 | −0.16 |
| GC | 0.08 | 0 | 0.05 | 0.09 |
| GG | 0.83 | 0.08 | 0.14 | 0.08 |
| wwbb1277 | | | | |
| GG | −1.3 | 0.19 | | |
| TG | 0.55 | 0.02 | 0.53 | 0.34 |
| TT | −0.28 | −0.01 | −0.28 | −0.18 |
| wwbb1332 | | | | |
| TC | 0.62 | 0.02 | 0.52 | 0.34 |
| TT | −0.29 | −0.01 | −0.27 | −0.18 |
| wwbb1373 | | | | |
| AA | −0.36 | −0.03 | −0.26 | −0.16 |
| AG | 0.66 | 0.05 | 0.51 | 0.31 |
| GG | 0.58 | 0.1 | | |
| wwbb1441 | | | | |
| CC | 3.82 | −0.09 | | |
| TC | 0.71 | 0.04 | 0.26 | 0.26 |
| TT | −0.3 | −0.01 | −0.11 | −0.11 |
| wwbb1444 | | | | |
| AA | −7.05 | −0.84 | | |
| GA | 1.02 | 0.07 | 0.78 | 0.54 |
| GG | −0.31 | −0.02 | −0.23 | −0.16 |
| wwbb181 | | | | |
| AA | 1.29 | 0.11 | 0.81 | 0.6 |
| GA | −0.35 | 0.02 | −0.24 | −0.18 |
| GG | −0.84 | −0.09 | −0.61 | −0.44 |
| wwbb20 | | | | |
| AA | 9.14 | 0.56 | | |
| GA | 1.04 | 0.07 | 0.46 | 0.38 |
| GG | −0.64 | −0.04 | −0.31 | −0.25 |
| wwbb244 | | | | |
| CC | 0.83 | 0.08 | 0.48 | 0.43 |
| TC | 0.09 | 0 | 0.01 | 0 |
| TT | −0.73 | −0.05 | −0.32 | −0.27 |
| wwbb289 | | | | |
| GG | −0.1 | −0.01 | −0.1 | −0.08 |
| GT | 0.71 | 0.12 | 0.78 | 0.63 |
| wwbb378 | | | | |
| AA | 1.07 | 0.1 | 0.73 | 0.54 |
| GA | −0.51 | −0.04 | −0.27 | −0.2 |
| GG | −0.03 | −0.03 | −0.06 | −0.02 |
| wwbb411 | | | | |
| AA | 0.26 | 0.03 | 0.12 | 0.09 |
| AT | −0.25 | −0.02 | −0.12 | −0.09 |
| TT | 0.03 | −0.03 | 0.03 | 0.1 |
| wwbb623 | | | | |
| CC | −0.55 | −0.03 | −0.45 | −0.36 |
| CT | −0.4 | −0.06 | −0.06 | −0.09 |
| TT | 0.54 | 0.08 | 0.17 | 0.19 |
| wwbb944 | | | | |
| AA | −0.68 | −0.05 | −0.2 | −0.2 |
| AC | 1.33 | 0.11 | 0.38 | 0.39 |
| CC | 1.57 | 0.02 | | |
| XWB111 | | | | |
| AA | −0.23 | −0.01 | −0.01 | 0.03 |
| AG | 0.47 | 0.01 | 0.01 | −0.07 |
| GG | 0.43 | 0.1 | 2.59 | 2.81 |
| XWB137 | | | | |
| AA | 3.5 | −0.39 | | |
| GA | 1.02 | 0.09 | 0.44 | 0.3 |
| GG | −0.48 | −0.04 | −0.26 | −0.18 |
| XWB291 | | | | |
| CC | 1.65 | 0.19 | 1.24 | 1.12 |
| CG | | | | |
| TC | 0.03 | 0.01 | 0.1 | 0.05 |
| TG | 26.05 | 0.62 | | |
| TT | −0.65 | −0.08 | −0.62 | −0.5 |
| XWB392 | | | | |
| CC | | | | |
| TC | 1.04 | 0.1 | 0.5 | 0.33 |
| TT | −0.48 | −0.04 | −0.3 | −0.19 |

(9) Soluble solid content:

| ddy19 | | HB15 | | s1_1 | |
|---|---|---|---|---|---|
| CC | 0.06 | AA | −0.28 | CC | 0 |
| CT | −0.04 | AG | 0.17 | CT | −0.04 |
| TT | −0.42 | GG | −0.01 | TT | 0.09 |
| s2_6 | | s5_42 | | s6_75 | |
| AA | −0.05 | AA | 0.03 | CC | −0.06 |
| TA | 0.08 | GA | −0.11 | CT | 0.14 |
| TT | −0.13 | GG | 0.04 | TT | −1.65 |
| s7_27 | | s8_17 | | XDY354 | |
| GG | 0.07 | CC | −1.2 | CC | −1.7 |
| GT | −0.04 | T/insAC | −1.63 | TC | −0.23 |
| TT | −0.49 | TC | 0.04 | TT | 0.12 |
| TT | −0.05 | | | | |
| XDY359 | | f10_114 | | f2_1 | |
| AA | −0.17 | AA | −0.12 | GG | −0.36 |
| AT | 0.05 | AG | 0.08 | GT | −0.03 |
| TT | 0.06 | GG | 0.2 | TT | 0.54 |
| f4_1 | | f5_3 | | f6_25 | |
| AA | −0.29 | GG | 0.02 | CC | −0.44 |
| GA | 0.15 | GT | 0 | TC | 0.11 |
| GG | −0.12 | TT | −1.19 | TT | 0.17 |
| f8_14 | | NNEVE | | SU201 | |
| AA | 0.12 | CC | −2.96 | AA | −0.24 |
| AT | 0.11 | TC | −0.01 | TA | 0.07 |
| TT | −0.4 | TT | 0.06 | TT | 0.05 |
| SU203 | | SU204 | | XDY368 | |
| CC | 0.16 | AA | −3.84 | AA | 0.36 |
| TA | −0.63 | AG | −2.67 | AG | 0.13 |
| TC | 0.17 | CA | −2.03 | GG | −0.24 |
| TT | −0.09 | CC | −0.14 | | |
| CG | 0.32 | | | | |
| GG | 0.4 | | | | |
| DYS17 | | TSS202 | | TSS203 | |
| AA | 0.05 | CC | 0.04 | CC | 0.16 |
| AG | −0.1 | CT | −0.17 | TC | −0.03 |
| GG | −1.62 | TT | −1.76 | | |
| TSS206 | | TSS209 | | TSS210 | |
| AA | −0.14 | CC | 0.07 | AA | −0.05 |
| AG | 0.2 | CT | −0.04 | AC | 0.31 |
| GG | −0.16 | TT | −0.08 | CC | 0 |
| TSS212 | | TSS213 | | TSS214 | |
| TA | −0.22 | CC | 0.36 | CC | −0.11 |
| TT | 0.1 | CT | −0.09 | CT | 0.32 |
| TT | −0.34 | TT | −0.88 | | |
| TSS215 | | TSS216 | | TSS217 | |
| CC | 0.04 | CC | 0.02 | CC | 0.05 |
| CT | −0.01 | CT | 0.05 | TC | −0.06 |
| TT | −0.59 | TT | −0.12 | TT | 0.02 |
| TSS218 | | TSS219 | | TSS221 | |
| GG | 0.17 | CC | −2.49 | insC/insCTCC | −3.27 |
| GT | −0.15 | TC | 0.18 | insCTCC | −0.96 |
| TT | −0.63 | TT | −0.09 | T/insCTCC | 0.25 |
| TA | 4.64 | | | | |
| TT | −0.07 | | | | |
| TSS222 | | TSS223 | | TSS225 | |
| AA | 0 | CC | −0.05 | CT | |
| AG | 0.07 | TC | 0.06 | GG | 0.14 |
| GG | −0.1 | TT | 0.03 | GT | 0.01 |
| TT | −0.22 | | | | |
| TSS226 | | TSS228 | | TSS229 | |
| AA | −0.1 | CC | 0.1 | AA | −0.02 |
| AG | 0.06 | TC | 0.12 | AG | 0.01 |
| GG | −0.47 | TT | −0.16 | GG | 0.01 |
| TSS232 | | TSS233 | | TSS234 | |
| AA | 0.09 | AA | 0.19 | AA | 0.08 |
| CA | −0.2 | GA | −0.27 | delGT | |
| CC | −0.43 | GG | −0.18 | G/delGT | 0.56 |
| GA | −0.29 | | | | |
| GG | 0.07 | | | | |
| TSS235 | | TSS236 | | TSS237 | |
| AA | −0.58 | AA | −0.41 | AA | −0.04 |
| AC | 0.04 | AG | −0.14 | AG | −0.02 |
| CC | 0.09 | GG | 0.42 | GG | 0.18 |
| TSS238 | | TSS240 | | TSS241 | |
| CC | 0.09 | CC | 0.03 | AA | −0.27 |
| TC | −0.05 | TC | −0.08 | AG | 0.18 |
| TT | −0.41 | TT | 0.15 | GG | 0.09 |
| GT | 0.15 | | | | |
| TT | 0.15 | | | | |
| TSS242 | | TSS245 | | TSS247 | |
| AA | −0.02 | CC | 0 | CC | 0 |
| AG | −0.1 | CT | 0.05 | CT | 0.1 |
| GG | 0.24 | TT | −0.07 | TT | −0.65 |
| TSS248 | | TSS249 | | TSS250 | |
| GG | 0.29 | AA | −0.08 | CC | −0.15 |
| TG | 0.03 | AG | 0.01 | CG | 0.03 |
| TT | −0.13 | GG | 0.06 | GG | 0.33 |
| TSS251 | | TSS252 | | TSS253 | |
| CC | 0.32 | AA | −0.19 | AA | 0.13 |
| TC | −0.12 | GA | 0.12 | GA | 0.14 |
| TT | −0.03 | GG | −0.08 | GG | −0.2 |
| TSS254 | | TSS255 | | TSS258−4 | |
| CC | −2.85 | CC | −0.18 | AA | −0.46 |
| GG | 0.09 | CT | 0.16 | GA | 0.01 |
| GT | 0.03 | TT | 0.49 | GG | 0.02 |
| TT | −0.36 | | | | |
| TSS260 | | TSS261 | | TSS263 | |
| AA | −0.18 | CC | 0 | AA | 0.56 |
| CA | 0.05 | CT | −1.31 | CA | 0.08 |
| CC | 0.11 | GC | 0.1 | CC | −0.29 |
| | | GG | −0.05 | C/M3 | −0.03 |
| | | GT | −1.47 | C/M4 | 0.32 |
| | | | | C/N4 | −2 |
| | | | | C/N3′ | 2.2 |
| TSS264 | | TSS266 | | | |
| CC | 0.38 | AA | −0.4 | | |
| TC | 0.07 | AC | 0.06 | | |
| TT | −0.14 | CC | 0.23 | | |
| TSS267 | | TSS268 | | TSS269 | |
| AA | 0.25 | AA | 0.15 | CC | 0.42 |
| GA | −0.11 | CA | 0.17 | TC | −0.14 |
| GG | −0.54 | CC | −0.53 | TT | −0.08 |
| TSS271 | | TSS272 | | TSS274 | |
| AA | −0.1 | | | AC | 1 |
| TA | 0.23 | TC | −0.04 | CC | 0.22 |
| TT | −0.16 | TT | 0.09 | TA | −2.85 |
| TC | 0.03 | | | | |
| TT | −0.36 | | | | |
| UDY1 | | TSS258-9 | | | |
| AA | −0.63 | CC | −0.09 | | |
| GA | 0.29 | TC | 0.03 | | |
| GG | −0.43 | TT | 0.01 | | |

(10) Spur tree architecture:

| | | | | wwl9 | |
|---|---|---|---|---|---|
| neww45 | | S1234 | | (neww45 ≠ CC) | |
| CC | −0.29 | TA | 0.11 | AA | 0.1 |
| TC | 0.04 | TT | −0.18 | AG | −0.09 |
| TT | 0.22 | | | | |
| ww19 (S1234 = TT; | | ww19 (S1234 = TA: | | S1246 | |
| neww45 = CC) | | neww45 = CC) | | (neww45 = CC) | |
| AA | −0.17 | AA | −0.03 | C/delC | 0.04 |
| AG | 0.15 | AG | 0.03 | CC | −0.03 |
| S1246 | | S1246 | | zxx57 | |
| (neww45 = TC) | | (neww45 = TT) | | (neww45 = CC) | |
| C/delC | −0.07 | C/delC | 0.16 | del/ins | 0.12 |
| CC | 0.06 | CC | −0.14 | ins/ins | −0.13 |
| zxx57 | | | | | |
| (neww45 ≠ CC) | | | | | |
| del/ins | −0.08 | | | | |
| ins/ins | 0.09 | | | | |

The present disclosure further provides a primer combination for marker PCR amplification when marker genotyping, including a primer combination shown in SEQ ID NO. 1 to SEQ ID NO. 638.

The present disclosure provides a marker genotyping protocol, including the following steps:

1) extracting a genomic DNA of a Malus sample to be tested;

2) conducting multiplex polymerase chain reaction (PCR) amplification on the genomic DNA sample using the primer combination to obtain an amplified product; and

3) genotyping of the amplified product by next-generation sequencing, to obtain a genotype of a Malus sample to be tested.

Preferably, a reaction system of the multiplex PCR amplification in step 2), calculated in 30 μL, may include the following components: 8 μL of the primer combination, 8 μL of MP004_Cu Panel Mix, 50-200 ng of DNA, 10 μL of 3×T enzyme and H₂O as a balance; each primer in the primer combination may have a concentration of 0.24 μM; and a reaction program of the multiplex PCR amplification may include: 95° C. for 3 min; 95° C. for 30 s, and 60° C. for 4 min, conducting 16 cycles; and extension at 72° C. for 4 min.

Preferably, the next-generation sequencing in step 3) may have a depth of 1200×.

The present disclosure provides a method for determining a trait phenotype or calculating a genomics-predicted phenotype value of a trait of the Malus sample, including the following steps:

obtaining a population average phenotype of a trait corresponding to the molecular marker as follows: fruit ripening date 159.45 DAFB, fruit cover color degree 56.35%, fruit weight 106.63 g, soluble solid content 14.85%, fruit juice pH value 3.34, fruit malate content 5.83 mg/mL, flesh firmness at harvest 12.18 kg/cm², flesh crispness at harvest 1.31 kg/cm², flesh firmness retainability 2.41 months, flesh crispness retainability 2.19 months, Fruit ring rot disease resistance 21.34 mm, and spur tree architecture 0.99;

obtaining a genomics predicted phenotype value of a Malus sample to be tested by the method; according to the genotype of the Malus sample to be tested, and according to the population average and the genotype effect value or the genotype combination effect value, determining a genomics predicted phenotype value for a trait of the Malus sample to be tested using the following criteria or calculating a predicted phenotype value using the following prediction model; where

the standard comprises:

(1) resistance to Glomerella leaf blotch:

when a genotype of S1202 is CC and a genotype of zhwy64 is CC, it is determined as disease-resistant; other genotypes are determined as susceptible;

(2) fruit shape:

when a genotype of newdy202 is CC, a genotype of SIZE2270 is GG, a genotype of SIZE5253 is CC, a genotype of SIZE9100 is GG or a genotype of SIZE9195 is AA, the fruit shape is determined to be conical-round;

when a genotype of SP031 is CC, a genotype of SP081 is not CT or a genotype of XDY231 is GG, the fruit shape is determined to be oblate-round;

the prediction model comprises:

(3) a chlorogenate content or a procyanidin B2 content adopts a genotype combination model;

an effect value is estimated according to a genotype combination of molecular markers for chlorogenate content or procyanidin B2 content, and a prediction model is established using a genotype combination effect value, with a formula as follows:

GPV=α×(GcE+μ)+β; where

GPV is a genomics predicted phenotype value; GcE is a genotype combination effect value of markers of the trait; μ is a mean of a phenotype of the trait in a training population; and α and β are a linear regression coefficient and a residual parameter, respectively;

(4) Fruit ripening date, soluble solid content, fruit juice pH, flesh firmness at harvest, flesh crispness at harvest, flesh firmness retainability, flesh crispness retainability, or fruit ring rot disease resistance adopts an additive model, with a formula as follows:

$$G P V = α \times ( {\sum }_{i = 1}^{k} G E + μ ) + β$$

where GPV is the genomics predicted phenotype value; GE is a genotype effect value of the marker; k is a number of markers for the trait; μ is the mean of a phenotype of the trait in the training population; and α and β are the linear regression coefficient and the residual parameter, respectively;

fruit weight, malate content, fruit cover color degree and spur tree architecture adopt a fixed-effect model, with a prediction formula as follows:

$$G P V = α \times ( Fx + γ \times {\sum }_{i = 1}^{k} GnE + μ ) + β$$

where GPV is the genomics predicted phenotype value; Fx is a fixed genotype effect value of a fixed-effect marker; GnE is a genotype effect value of a non-fixed-effect marker of the trait; k is a number of markers for a non-fixed-effect of the trait; μ is the mean of a phenotype of the trait in a training population; γ is a shrinkage factor; and α and β are the linear regression coefficient and the residual parameter, respectively;

a fixed-effect of the fruit weight is as follows: a genotype of XDY160 is AA, or a genotype of SIZE4849 is GG or a genotype of SIZE4161 is GG, Fx is −104.8, −100.7 and −101.4, respectively;

a fixed-effect of fruit malate content is a genotype combination effect value of Ma, MA202 and SAUR-5;

a fixed-effect of fruit cover color degree is a genotype combination effect value of ZZZ162 with zwy6, and ZZZ162 with color1245;

a fixed-effect of spur tree architecture is a genotype combination effect value of neww45 with S1245, and neww45 with ww19.

The present disclosure further provides following one or more uses of the Malus trait-associated molecular marker, or the genotype effect value or the genotype combination effect value, or the primer combination:

1) Trait phenotype prediction of Malus; 2) construction of fingerprint or molecular ID card of Malus; 3) genotype identification of Malus germplasm accessions; 4) hybrid breeding of Malus; and 5) molecular distinctness, uniformity and stability (DUS) test of new cultivars of Malus; where

the hybrid breeding of Malus comprises one or more of selection of hybrid parental materials and cross combinations, design of hybrid generations, and molecular-assisted selection of hybrids.

The present disclosure provides a Malus trait-associated molecular marker. There are a total of 319 molecular markers, including 318 single-nucleotide polymorphism (SNP) markers and 1 InDel marker; and the molecular markers are related to 16 traits including fruit ripening date, fruit shape, fruit cover color degree, fruit weight, soluble solid content, fruit juice pH value, malate content, chlorogenate content, procyanidin B2 content, flesh firmness at harvest, flesh crispness at harvest, flesh firmness retainability, flesh crispness retainability, fruit ring rot disease resistance, Glomerella leaf blotch resistance, and spur tree architecture. The molecular markers of the present disclosure can be used for apple germplasm resource evaluation and breeding, can greatly improve apple breeding efficiency and shorten breeding cycle.

### DETAILED DESCRIPTION OF THE EMBODIMENTS

The present disclosure provides a Malus trait-associated molecular marker, including one or more of molecular markers in the Table 1.

In the present disclosure, the molecular marker includes 318 SNP markers and 1 InDel marker.

In the present disclosure, the SNP molecular marker has a version number based on a apple genome sequence information of GDDH13 v1.1.

The present disclosure further provides a genotype effect value or a genotype combination effect value of each genotype of the molecular marker on a corresponding trait as shown in the Table 2.

The present disclosure further provides a primer combination for detecting the molecular marker, including a primer combination shown in SEQ ID NO. 1 to SEQ ID NO. 638. In the present disclosure, marker names, upstream primers and downstream primers corresponding to the primer combination are shown in Table 3.

TABLE 3
Primer combinations for detecting different
molecular markers

| Marker | upstream primer | downstream primer |
|---|---|---|
| TSS202 | agataaatgactcgg | tgtctccagattgcg |
| | caagttcaaa | aaagataatc |
| | SEQ ID NO: 1 | SEQ ID NO: 2 |
| TSS203 | tgccatcttccggtt | ccgatagagaatgtc |
| | gaatt | ttgctacgt |
| | SEQ ID NO: 3 | SEQ ID NO: 4 |
| LY064 | attttagctcttccc | tgtgcgcaatgtttg |
| | aaaagtacgac | aattttgaagt |
| | SEQ ID NO: 5 | SEQ ID NO: 6 |
| TSS206 | tgtccactctttcgt | tgtagctaatctcta |
| | tccaaaaatgaag | aactttaaagtcgat |
| | SEQ ID NO: 7 | tat |
| | | SEQ ID NO: 8 |
| SP011 | gaaggggcacggcac | actgaccttggacca |
| | aac | tggggcct |
| | SEQ ID NO: 9 | SEQ ID NO: 10 |
| XDY10 | tgaagtagctctttc | aggaaagttgatgtg |
| | atcaaaccc | gactcgaatga |
| | SEQ ID NO: 11 | SEQ ID NO: 12 |
| s7_27 | tcgaaatttgttgag | ggtcagtcttttctg |
| | tcattg | tacatctgcagg |
| | SEQ ID NO: 13 | SEQ ID NO: 14 |
| SU203 | ccttgagacacaacc | tgagagtggatatct |
| | ctataccaactg | agggtttcgt |
| | SEQ ID NO: 15 | SEQ ID NO: 16 |
| s8_17 | caaagtgtattaagt | gaaggaaatcgaggg |
| | ttgtgtcagcact | aaaataga |
| | SEQ ID NO: 17 | SEQ ID NO: 18 |
| TSS209 | gtgcaaatttataaa | acaggaagcaacgaa |
| | aatattgatattgat | aagaatccaattcaa |
| | ag | c |
| | SEQ ID NO: 19 | SEQ ID NO: 20 |
| f2_1 | ataaatgataaaaac | attaggtgatctgat |
| | cccggcaaatc | tcggtggatg |
| | SEQ ID NO: 21 | SEQ ID NO: 22 |
| NNENE21 | gtaagcgtgcacaac | aagggtgaacgagta |
| | ttcaattaacaat | taattagcaac |
| | SEQ ID NO: 23 | SEQ ID NO: 24 |
| SZ8111 | tcgtaaaaattttgc | caaaaagaccaagaa |
| | caacccaagcaa | gcccaaaca |
| | SEQ ID NO: 25 | SEQ ID NO: 26 |
| TSS210 | ttgccttgcttccaa | tctacccaagttacc |
| | atattgaacat | ttgccaatagc |
| | SEQ ID NO: 27 | SEQ ID NO: 28 |
| LL01502 | cttttcctgctctcc | tgatcttgtcccttg |
| | ttcaca | tcgttgatataccg |
| | SEQ ID NO: 29 | SEQ ID NO: 30 |
| SU204 | cagtaaaacgacatg | attcatgcatcgtct |
| | gtccaattgatgca | tgttcttgttag |
| | SEQ ID NO: 31 | SEQ ID NO: 32 |
| SZ8165 | agcgtacatggcttt | tcaattcccactctc |
| | tatggtatgag | caaaagttt |
| | SEQ ID NO: 33 | SEQ ID NO: 34 |
| LY284 | gaagcaaaacctgtc | tggactgagattttt |
| | ccataa | caagcca |
| | SEQ ID NO: 35 | SEQ ID NO: 36 |
| MY154 | attaatgccatgcac | caaatagctaacaag |
| | ataatttcac | aacagtggt |
| | SEQ ID NO: 37 | SEQ ID NO: 38 |
| TSS212 | gagcccaaactgtta | attcctcaattggtt |
| | ttgcaaa | tgtctgcgtt |
| | SEQ ID NO: 39 | SEQ ID NO: 40 |
| TSS21302 | attgagtgtcaatga | tcagggggtcaaacg |
| | accaaagatatactt | acaatt |
| | g | SEQ ID NO: 42 |
| | SEQ ID NO: 41 | |
| f41 | agacagcaaacgact | tctgcaagaacatct |
| | aaaagag | tcacagttctgaa |
| | SEQ ID NO: 43 | SEQ ID NO: 44 |
| TSS214 | tgcctagtcttgaag | atatgaggatccttt |
| | cagtccatg | aatttgtggtgcc |
| | SEQ ID NO: 45 | SEQ ID NO: 46 |
| TSS215 | gttctaataaaaggg | gggcctgatgatatt |
| | gtggtcc | atgggttttgattg |
| | SEQ ID NO: 47 | SEQ ID NO: 48 |
| f5_3 | ttttatttaagtgac | ctctctcctctatct |
| | gggtacagctcag | caaacccaaag |
| | SEQ ID NO: 49 | SEQ ID NO: 50 |
| TSS216 | gtagtaaagttgcag | acattccttcactaa |
| | tctttaagtga | ttcctccctaaat |
| | SEQ ID NO: 51 | SEQ ID NO: 52 |
| S710 | ataaagtaattggtg | aaacatcaatcctac |
| | gaacctcca | attccattcacg |
| | SEQ ID NO: 53 | SEQ ID NO: 54 |
| S733 | accaaaacgagtcaa | attttccaagtcgtc |
| | agttca | tcgttt |
| | SEQ ID NO: 55 | SEQ ID NO: 56 |
| wwbb1444 | tttcctggatctgct | cgaggtaagaggata |
| | ggatttt | tacttg |
| | SEQ ID NO: 57 | SEQ ID NO: 58 |
| SZ3463 | gtggatgaggccatg | ctcccaaaagttagg |
| | actctaagagaa | gcaagataagg |
| | SEQ ID NO: 59 | SEQ ID NO: 60 |
| wr301 | cttgactttttcctt | ggaagcggctctgaa |
| | atcgtctt | gactagtattagg |
| | SEQ ID NO: 61 | SEQ ID NO: 62 |
| SZ3571 | caaacgactttagtc | acatcatggagagta |
| | tttcaatagttgagt | tttcctcc |
| | t | SEQ ID NO: 64 |
| | SEQ ID NO: 63 | |
| TSS217 | ctaatatagctggat | accaaatggatgaat |
| | ttaatgt | tttggagt |
| | SEQ ID NO: 65 | SEQ ID NO: 66 |
| SZ8286 | ccgaacggagaaatt | ttaaagttgttcaag |
| | cttcataagg | agtttctcgg |
| | SEQ ID NO: 67 | SEQ ID NO: 68 |
| zwyx71 | tgaaatcatgctata | aactactaactgagg |
| | tttagatgtacttt | agaatgtgcgc |
| | SEQ ID NO: 69 | SEQ ID NO: 70 |
| XDY32 | gcttcagttcataga | aaactcagagatggg |
| | ggagacgaccaggaa | atgaga |
| | c | SEQ ID NO: 72 |
| | SEQ ID NO: 71 | |
| ddy6 | gaagaagtacgagct | caacaacactgaggc |
| | tgtcataacaac | attgtcaat |
| | SEQ ID NO: 73 | SEQ ID NO: 74 |
| SZ3790 | gcataagtgatttaa | cattggaaatgtggt |
| | tccaccctc | gcttatgagtaat |
| | SEQ ID NO: 75 | SEQ ID NO: 76 |
| wwbb1063 | gaagatgaggcatgc | tgggggaccgataag |
| | ttaaaagattcc | tctttagattaat |
| | SEQ ID NO: 77 | SEQ ID NO: 78 |
| SZ839702 | tttcgatcatgaagg | cgaaagcagaggcac |
| | ttaggaagg | tttatatcc |
| | SEQ ID NO: 79 | SEQ ID NO: 80 |
| SZ12448 | attcacgcttcatct | gaaaaggggcatgca |
| | ctttcctaagatagg | aaaatcattttectt |
| | SEQ ID NO: 81 | SEQ ID NO: 82 |
| SZ8558 | aatcttgagtgatgg | gataaagggctgtag |
| | gaagagg | atccttcca |
| | SEQ ID NO: 83 | SEQ ID NO: 84 |
| SZ4161 | cgtttgttgaagctg | catgctttgttcgat |
| | agtataggggt | catgttgtaccaaa |
| | SEQ ID NO: 85 | SEQ ID NO: 86 |
| f6_25 | tgatgaaatgagagg | tcgaacttgtcccta |
| | aacatggctg | cagttcagagca |
| | SEQ ID NO: 87 | SEQ ID NO: 88 |
| newdy202 | gccaaatttaaaatt | aaaggcacttacaaa |
| | tagcaagttttgggc | caagtcataaaacaa |
| | tcg | atg |
| | SEQ ID NO: 89 | SEQ ID NO: 90 |
| XDY50 | ccgtacatttcccag | tgtccagtctctctg |
| | ttattag | taatactctggaa |
| | SEQ ID NO: 91 | SEQ ID NO: 92 |
| f8_14 | tagattaaggagctg | agtagttttcaacct |
| | acagagttcg | atccatcaccggtt |
| | SEQ ID NO: 93 | SEQ ID NO: 94 |
| XDY368 | tgaaaagattcacca | gctcagtttgctctt |
| | aatcccagcc | ggagtacttgatggc |
| | SEQ ID NO: 95 | gc |
| | | SEQ ID NO: 96 |
| zwyx448 | ttctcccatactgat | attgccaaagctatt |
| | catgtgact | attggtaatga |
| | SEQ ID NO: 97 | SEQ ID NO: 98 |
| SP031 | gctgaaattctaact | cagtgtcaagctatg |
| | ctgtttgacatc | gcaaatg |
| | SEQ ID NO: 99 | SEQ ID NO: 100 |
| SP032 | aattggttcaaaatc | tttttctttgataaa |
| | ctcaaaggcctt | atctatggcagg |
| | SEQ ID NO: 101 | SEQ ID NO: 102 |
| NBDY40 | ccgaggtgctacttg | ttgtaccaatgtcct |
| | attgttgcaaa | acaaccgtgg |
| | SEQ ID NO: 103 | SEQ ID NO: 104 |
| zxxl25 | aagaagtagagggag | ttttctgggtgtttt |
| | agagagagcca | aatgcg |
| | SEQ ID NO: 105 | SEQ ID NO: 106 |
| neww378 | attacgttcttgata | gcaagatgagtttta |
| | tgcggattt | atgaaccc |
| | SEQ ID NO: 107 | SEQ ID NO: 108 |
| S391 | gaagcatcattatca | atatagaaccacagg |
| | cacgaatcag | aggaatgttg |
| | SEQ ID NO: 109 | SEQ ID NO: 110 |
| XLCO42 | ataaatattaaatag | ccctcactegttccc |
| | tctaaggggtg | ttcttttcttttctt |
| | SEQ ID NO: 111 | t |
| | | SEQ ID NO: 112 |
| LL984 | aatgatgtttgtctt | atcgtgacttgggaa |
| | cataatcaccccca | acaattctttc |
| | SEQ ID NO: 113 | SEQ ID NO: 114 |
| WBBBB219 | aaaacataacgaggt | tgacatagtggcaaa |
| | gattatggggtcgta | taagaggtt |
| | | SEQ ID NO: 116 |
| | SEQ ID NO: 115 | |
| DDDD2 | ctagaacgtctagaa | agatcgacaagtatt |
| | aaccaggatcc | cttcgcttacaact |
| | SEQ ID NO: 117 | SEQ ID NO: 118 |
| CY242 | tccaaaatttgcttc | tttcgacatttggat |
| | ataagacacagt | ttgaaagttta |
| | SEQ ID NO: 119 | SEQ ID NO: 120 |
| CY564 | ttcaaacagaggtaa | agtagaggtgtttaa |
| | cctgag | ggtgagatgc |
| | SEQ ID NO: 121 | SEQ ID NO: 122 |
| XDY103 | tctttccattggttt | ctataacgccatttc |
| | gacactgatagattg | ttcttctgtgttggt |
| | SEQ ID NO: 123 | SEQ ID NO: 124 |
| S39502 | tctttgacgagaatc | catgggtaatgtttt |
| | gaatctaagacctc | gttttgagtgc |
| | SEQ ID NO: 125 | SEQ ID NO: 126 |
| CY603 | ttaaacccaagacca | ctcatagctgcatat |
| | agcataagtct | ttcgtcgttg |
| | SEQ ID NO: 127 | SEQ ID NO: 128 |
| TSS218 | acctcaaaatttgtc | aaatcccagatcaga |
| | tggttga | atttttcccaaaaaa |
| | SEQ ID NO: 129 | |
| | | SEQ ID NO: 130 |
| GWBI | aatacaccgctcttg | agatcactctaccag |
| | aataatttcccc | gtacgacaaatttcc |
| | SEQ ID NO: 131 | aa |
| | | SEQ ID NO: 132 |
| CY874 | aagggtcaatttgtt | gcgttaattatttgt |
| | aaatcttatcatgac | tgcttatttcaattt |
| | a | |
| | SEQ ID NO: 133 | SEQ ID NO: 134 |
| XLC218 | tttgatgacgatgaa | ttggatcaaactaag |
| | aacttctgggatc | agaatgcagcaaa |
| | SEQ ID NO: 135 | SEQ ID NO: 136 |
| WBBB71 | tttgaaaactgctgt | gaagttgaacatggt |
| | cagattcttgtga | gtggtttgaaa |
| | SEQ ID NO: 137 | SEQ ID NO: 138 |
| XLC556 | tagaggaaaccatct | aggtatctggttcct |
| | tcgtctttgttttc | gctgatttgactgtt |
| | SEQ ID NO: 139 | g |
| | | SEQ ID NO: 140 |
| nc91 | tgccattttcagagt | tccatagcattatta |
| | gcaatatgcta | cggtttgtgcttga |
| | SEQ ID NO: 141 | SEQ ID NO: 142 |
| XWB321 | aaaatgaggatatat | ctcatcccagatatg |
| | tcacgcaca | acatggcat |
| | SEQ ID NO: 143 | SEQ ID NO: 144 |
| LL1499 | tggccacaaaggaaa | cctccagcatggtta |
| | aatatccacata | ttaacaaga |
| | SEQ ID NO: 145 | SEQ ID NO: 146 |
| S372 | gaatctggaccaaca | aggctctgaataatc |
| | ctgatagtcaa | tcgttt |
| | SEQ ID NO: 147 | SEQ ID NO: 148 |
| neww278 | gcgatatgttgacaa | ggtgcacatatcaaa |
| | tttctccaat | gaatctgctaca |
| | SEQ ID NO: 149 | SEQ ID NO: 150 |
| GWB636 | gtgtatactctatgt | taagaataatcctat |
| | gtaatttctcctta | gttgca |
| | SEQ ID NO: 151 | SEQ ID NO: 152 |
| LL288 | tggaagtttgtcgat | caacgtgtgtgatat |
| | atgcctttgt | ttgagtct |
| | SEQ ID NO: 153 | SEQ ID NO: 154 |
| WBBBB13 | ctcgttggatgtcgg | gctttaacactctct |
| | attctg | cctcgctata |
| | SEQ ID NO: 155 | SEQ ID NO: 156 |
| LL531 | ctttctgggtaggaa | gagaccaaactcttt |
| | taacacgatttgtt | tctcttgt |
| | SEQ ID NO: 157 | SEQ ID NO: 158 |
| WBBBB85 | tttataagacgaaga | aaagtcaacatcttg |
| | ccccttga | ttcgcg |
| | SEQ ID NO: 159 | SEQ ID NO: 160 |
| LL796 | gttccagtgacatca | aaattgaaggatgtc |
| | agcataagtctc | catctttac |
| | SEQ ID NO: 161 | SEQ ID NO: 162 |
| S477 | tttgggaattattcg | caccgtacttggtta |
| | ttgtttcgca | tgtccagtat |
| | SEQ ID NO: 163 | SEQ ID NO: 164 |
| MY355 | ttgatcctctactct | aaagttgaaagagga |
| | cgaagaccaagatca | tgctggccc |
| | tc | SEQ ID NO: 166 |
| | SEQ ID NO: 165 | |
| neww45 | cattctgttgggttt | gagaccgactcaaca |
| | ccgc | atggtagt |
| | SEQ ID NO: 167 | SEQ ID NO: 168 |
| S484 | gttatgtgatgtatt | ctaacacacttggta |
| | atttcacgtgt | ttattatttcagt |
| | SEQ ID NO: 169 | SEQ ID NO: 170 |
| TSS219 | aattctctgtttgat | tttttgtaggcattt |
| | tgccat | gatcgggtt |
| | SEQ ID NO: 171 | SEQ ID NO: 172 |
| TSS221 | tcgtaagcaacacgg | agaagctgatgaatc |
| | cgaaaagatat | tcagtgtttctga |
| | SEQ ID NO: 173 | SEQ ID NO: 174 |
| TSS22202 | tccttcggaaagaga | atcgaataaatacca |
| | gagaaacgag | actcccacttggaat |
| | SEQ ID NO: 175 | SEQ ID NO: 176 |
| TSS223 | atgaccaataaatct | atctgcactgtacac |
| | agaacaagagtagca | gtgtatacatc |
| | SEQ ID NO: 177 | SEQ ID NO: 178 |
| TSS225 | tgcacatttccacta | aattaaactactgta |
| | accgaatatgtat | tgtgegect |
| | SEQ ID NO: 179 | SEQ ID NO: 180 |
| SP041 | ccagtcatactgact | ttgtaaatttcaatt |
| | gtagaggaacctcgt | ctcacc |
| | SEQ ID NO: 181 | SEQ ID NO: 182 |
| SP043 | tcaaggcacgattcc | ccctgaagttgattt |
| | taattggattt | gagtgaaggt |
| | SEQ ID NO: 183 | SEQ ID NO: 184 |
| SP044 | gccaaatttatgcca | ggttcgtacagaaat |
| | catggcaa | gggagca |
| | SEQ ID NO: 185 | SEQ ID NO: 186 |
| SP042 | atgtttatggcattg | gtcgaagtgaacatg |
| | ctgataca | taatagtgcca |
| | SEQ ID NO: 187 | SEQ ID NO: 188 |
| CY139902 | gagtcacacctaccg | ttgaagtaagggtag |
| | aactgttaggtag | attgtgt |
| | SEQ ID NO: 189 | SEQ ID NO: 190 |
| CY1591 | atttccataaacttc | cttctaaacccggca |
| | acaggcaatga | aactaagttctattg |
| | SEQ ID NO: 191 | SEQ ID NO: 192 |
| SP051 | taaccatgttgaaat | gatgtaacccttctt |
| | tctcgccctaca | gagctgttca |
| | SEQ ID NO: 193 | SEQ ID NO: 194 |
| zwy6 | aacgagatatcgaca | catgcatcaagaaga |
| | tgatttgt | agctca |
| | SEQ ID NO: 195 | SEQ ID NO: 196 |
| colorll69 | tgatgatgtcaaaac | aaatgttgcattctg |
| | aacacgcttaac | tagagctcat |
| | SEQ ID NO: 197 | SEQ ID NO: 198 |
| S1118 | ctcatatcctttcaa | tacataacctcctgt |
| | tgtccagc | cctgattgttcag |
| | SEQ ID NO: 199 | SEQ ID NO: 200 |
| LYY2825 | caagagcaattaacg | cgagtggtgtttttg |
| | ccatcctaattt | tttgattcttg |
| | SEQ ID NO: 201 | SEQ ID NO: 202 |
| s5_42 | aaatggccacatctt | tccagacatattttg |
| | caatttgca | agtgtcacttgtca |
| | SEQ ID NO: 203 | SEQ ID NO: 204 |
| SZ8646 | atgcagacataaggg | ccagaatacagtggg |
| | gaacaag | aacttctcaacg |
| | SEQ ID NO: 205 | SEQ ID NO: 206 |
| wrl97 | tggtttgctcactaa | gcacaaatcattttc |
| | attggatgg | gcgaagatgacaca |
| | SEQ ID NO: 207 | SEQ ID NO: 208 |
| TSS226 | atcacaacatatcca | ggatggttcttgtca |
| | attttgcattt | tttaagct |
| | SEQ ID NO: 209 | SEQ ID NO: 210 |
| zwyy29 | aaaacagttcgaagg | agtcttggaaacgaa |
| | ttggtct | gtacatct |
| | SEQ ID NO: 211 | SEQ ID NO: 212 |
| colorl31 | cagtttcaatatacc | cttttgtgaatccaa |
| | aaaggcagcagta | gtaaatgggg |
| | SEQ ID NO: 213 | SEQ ID NO: 214 |
| TSS228 | ggaatacaacttttc | cacccttttccttta |
| | caaccccaagtgcac | tgcttcatagaac |
| | SEQ ID NO: 215 | SEQ ID NO: 216 |
| color318 | cagcaaactcatgtt | tggagctgtcttgtg |
| | cttcaagtagctct | gcctaattaatcac |
| | SEQ ID NO: 217 | SEQ ID NO: 218 |
| mb44 | ctcgttaaactttgt | ttttggtcaaagtca |
| | tggataactct | tccgtttgcttacgc |
| | SEQ ID NO: 219 | |
| | | SEQ ID NO: 220 |
| MA202 | atggttgcatatgaa | caagttcttcaacat |
| | gaagcaactct | atcaagccca |
| | SEQ ID NO: 221 | SEQ ID NO: 222 |
| TSS229 | cttggaaatcccagt | ttatatagcttagag |
| | tgaggttgaaggg | ggctttcttacgggc |
| | SEQ ID NO: 223 | t |
| | | SEQ ID NO: 224 |
| s6_75 | accccatttttgttg | ggtgaggttagtgtg |
| | tggacc | gtcgaattctaat |
| | SEQ ID NO: 225 | SEQ ID NO: 226 |
| SAURO5 | agtgatgatgttcct | ggcttagagtatacc |
| | aagattgt | aaaataccg |
| | SEQ ID NO: 227 | SEQ ID NO: 228 |
| SAUR06 | ccaatgtgggatgaa | tcaatcacagttcgt |
| | tcaagaagcatt | ggctgttc |
| | SEQ ID NO: 229 | SEQ ID NO: 230 |
| SP081 | gttgagcgtatgaat | atggaggactataga |
| | cctatat | aagagaaaacgaaag |
| | SEQ ID NO: 231 | t |
| | | SEQ ID NO: 232 |
| SZ8824 | gctatgtttagggac | tgagatcacattaac |
| | aggaagt | atgtagc |
| | SEQ ID NO: 233 | SEQ ID NO: 234 |
| TSS232 | acatgtacgattcat | gtgcaaaggtgaaca |
| | gggcaacc | caggacaaca |
| | SEQ ID NO: 235 | SEQ ID NO: 236 |
| C400 | cgagtcagtttagga | catcatgtccacaag |
| | cccctatat | aaccctaa |
| | SEQ ID NO: 237 | SEQ ID NO: 238 |
| TSS233 | cttggtattatttac | tttacaaccaaaatc |
| | tttacgttttatttt | attgcagatgcggt |
| | gtctttat | SEQ ID NO: 240 |
| | SEQ ID NO: 239 | |
| TSS234 | cactcgctgcatctt | ttgtgaggggatcat |
| | aaactattatga | atctgtagtcgt |
| | SEQ ID NO: 241 | SEQ ID NO: 242 |
| TSS235 | actacacaaatggga | tacagagaccttctt |
| | tgagactttcca | ctcataacca |
| | SEQ ID NO: 243 | SEQ ID NO: 244 |
| TSS236 | gagttcgcgtattct | aaagtctttacatct |
| | acaattat | ccgcatgaatc |
| | SEQ ID NO: 245 | SEQ ID NO: 246 |
| TSS237 | ttectaagaaaccct | gatttgttcagacca |
| | agcttccattt | gtatgttcctt |
| | SEQ ID NO: 247 | SEQ ID NO: 248 |
| TSS238 | caaaatttctccttc | atgtgcctacaagaa |
| | aaactca | attaaatgggt |
| | SEQ ID NO: 249 | SEQ ID NO: 250 |
| f10_1149 | gegttgtcttccatg | aattttggccagaat |
| | attttcccaagt | acgaacaa |
| | SEQ ID NO: 251 | SEQ ID NO: 252 |
| TSS240 | tcgcttttagccaac | agaggaagaagaaaa |
| | actaattatt | catggggta |
| | SEQ ID NO: 253 | SEQ ID NO: 254 |
| TSS241 | aatagctagacaatt | aattccatctcaagt |
| | taatgagtttgt | gtcagttgccaa |
| | SEQ ID NO: 255 | SEQ ID NO: 256 |
| TSS242 | atgagtgagcgtttg | ccgcagcatcaatca |
| | gttttccgt | taattatatgcaact |
| | SEQ ID NO: 257 | |
| | | SEQ ID NO: 258 |
| ZZZ162 | acaaatatctctcgc | gttctctctttaatt |
| | atgtctttgtaatt | tcctggtgttgc |
| | SEQ ID NO: 259 | SEQ ID NO: 260 |
| TSS245 | aggttccaaatttaa | gegcagtgtaagagg |
| | atctccctcgca | aaaatgttga |
| | SEQ ID NO: 261 | SEQ ID NO: 262 |
| SU201 | aagaccaactagagt | tccataaaaaggaca |
| | ggatagctaacttta | caagacctg |
| | a | SEQ ID NO: 264 |
| | SEQ ID NO: 263 | |
| color852 | gtgggctcatataaa | accactaaatgtttg |
| | caacaaataag | atccttgtc |
| | SEQ ID NO: 265 | SEQ ID NO: 266 |
| neww26 | atctgctacagagaa | cacttgtgcaattca |
| | aaacggtaaaccatc | aaatttc |
| | t | SEQ ID NO: 268 |
| | SEQ ID NO: 267 | |
| wwbb1l59 | aattatcttgccgag | ccctagctcctttcg |
| | ctttttgtcct | acccttttaatgacc |
| | SEQ ID NO: 269 | |
| | | SEQ ID NO: 270 |
| wwbb1277 | tctgttctgtctcct | agagagtgaaggtag |
| | agtttgtatgg | ctccatc |
| | SEQ ID NO: 271 | SEQ ID NO: 272 |
| wwbb1332 | gtttctagtgtatgg | acgataatgtaactc |
| | gccaactc | agctca |
| | SEQ ID NO: 273 | SEQ ID NO: 274 |
| wwbb1200 | taggaggttcaaatt | tcatgagagtcttgt |
| | tgagatatttttcaa | atagggttggt |
| | c | SEQ ID NO: 276 |
| | SEQ ID NO: 275 | |
| wwbb1373 | ctaaaaaccatacca | aattttttcacaccg |
| | gtgaaccaacc | tttgaactcga |
| | SEQ ID NO: 277 | SEQ ID NO: 278 |
| S1122 | tgcagacacaacagt | ctggaatgtgtacca |
| | aagttctcc | aaatctcaaata |
| | SEQ ID NO: 279 | SEQ ID NO: 280 |
| Zzwyl02 | tttcaccaatggtct | gagaattcccclatt |
| | tatattcagaaagac | ttgaa |
| | ta | SEQ ID NO: 282 |
| | SEQ ID NO: 281 | |
| Hzwy486 | caaccaccatccgtg | aaagggacccaatat |
| | aacatataaac | tttgtttgtttgt |
| | SEQ ID NO: 283 | SEQ ID NO: 284 |
| SP101 | caacagaatgtccgt | atgagtatgcagacg |
| | tagaactcagatt | ggaaagaactag |
| | SEQ ID NO: 285 | SEQ ID NO: 286 |
| wwbb378 | gtgagagtgatggtc | tgctacattccccca |
| | aagtgataaga | aatttagtt |
| | SEQ ID NO: 287 | SEQ ID NO: 288 |
| wwbb411 | caaccaaatcaatga | aacgtgatcatgcat |
| | acaagggaagaata | ctgttgaag |
| | SEQ ID NO: 289 | SEQ ID NO: 290 |
| HB123 | tgaaaaaacatgaaa | gcgccttatacaagt |
| | gtcgcatgaatgt | gttatatggtg |
| | SEQ ID NO: 291 | SEQ ID NO: 292 |
| newdy33 | tgaagagacgtaagg | ctcaagtgttgcgaa |
| | atgcctcac | gatgatacag |
| | SEQ ID NO: 293 | SEQ ID NO: 294 |
| SZ1348 | aacgtaaatttgttt | gaacaataacttgcc |
| | ggtttgag | tgaggaaaagtctcc |
| | SEQ ID NO: 295 | a |
| | | SEQ ID NO: 296 |
| colorl245 | cctcatggagattca | aacgagctggtgtaa |
| | tttcccttect | gtttcctataaat |
| | SEQ ID NO: 297 | gg |
| | | SEQ ID NO: 298 |
| SZ832 | ctgagagacttggcc | ggtgcaaagccaatg |
| | aagaaatacgg | tttgtatcacc |
| | SEQ ID NO: 299 | SEQ ID NO: 300 |
| SZ906 | agaagatacgcggtt | aaaagtcacccctta |
| | tgtttt | aattggaagaggcg |
| | SEQ ID NO: 301 | SEQ ID NO: 302 |
| wwbb1441 | acaagactaccat | acttgaaaggcggga |
| | aagcccccttatg | aattgtatttgag |
| | cttc | SEQ ID NO: 304 |
| | SEQ ID NO: 303 | |
| Y323 | caaacgtgaaaatct | tggggttgttctaat |
| | gaggataagttccct | gtaatctgcagc |
| | g | SEQ ID NO: 306 |
| | SEQ ID NO: 305 | |
| LY591 | cactgegtgcatata | tattatttcttagat |
| | catacattcatta | tcacaggtttcg |
| | SEQ ID NO: 307 | SEQ ID NO: 308 |
| SZ4250 | caaggagttcctttc | actgaagtgttgctg |
| | attctcatccaaccc | aagaatgag |
| | | SEQ ID NO: 310 |
| | SEQ ID NO: 309 | |
| Y397 | ggggtgttcacatag | agaacaacctctttc |
| | gatttgtatg | atggca |
| | SEQ ID NO: 311 | SEQ ID NO: 312 |
| SZ4378 | agtgatacttgccat | tgtgtccaacgagat |
| | gtaggactctc | taggtgtg |
| | SEQ ID NO: 313 | SEQ ID NO: 314 |
| S1246 | gcatctagtccattc | gaggtcttatcatcc |
| | gtaatgtaggcgcca | tgtccatctccaga |
| | a | SEQ ID NO: 316 |
| | SEQ ID NO: 315 | |
| zxx51 | gcaaatctttgtatt | cttacccttggtttg |
| | ctgcttgcag | tactgtt |
| | SEQ ID NO: 317 | SEQ ID NO: 318 |
| SZ4457 | agttgtatcaaagtc | aacacatggaaagag |
| | acgaatca | attgatcacctt |
| | SEQ ID NO: 319 | SEQ ID NO: 320 |
| SZ4595 | gtaataaaaggagga | cattaaatgggtgga |
| | agtggctgcg | tgaggatgaccccg |
| | SEQ ID NO: 321 | SEQ ID NO: 322 |
| neww5 | tgtgaccagaccaaa | atgggtcttgaaata |
| | cggaaaaactctaga | ttaatgagattccgt |
| | t | tt |
| | SEQ ID NO: 323 | SEQ ID NO: 324 |
| CY1821 | cggcctatttttgct | gaaagtatgaaatgg |
| | aatggtatgct | aaccagtctttaa |
| | SEQ ID NO: 325 | SEQ ID NO: 326 |
| SZ12710 | gatagatggaagggg | agggtgggtatatta |
| | gaaaa | gcactctcaatta |
| | SEQ ID NO: 327 | SEQ ID NO: 328 |
| neww61 | tgacaccaaccattc | ttgcaaatggagagt |
| | aaaagtgctattg | ggtgtagtgt |
| | SEQ ID NO: 329 | SEQ ID NO: 330 |
| wr70 | gatgtaagcacagtt | ggcttctttttatca |
| | tttgtcgatcca | ccgagttgaaaacg |
| | SEQ ID NO: 331 | SEQ ID NO: 332 |
| neww16 | aaaatgacagactcg | agtagtgttactggc |
| | attgcagc | tctgtatcttgttaa |
| | SEQ ID NO: 333 | |
| | | SEQ ID NO: 334 |
| SZ9100 | aaccaagaaacaagt | attcagttgacatct |
| | catagct | gcgaaagga |
| | SEQ ID NO: 335 | SEQ ID NO: 336 |
| CY2464 | cgatttctcgactgg | aaatccagatctgaa |
| | ttaatcgatcatcg | agcacgagatg |
| | SEQ ID NO: 337 | SEQ ID NO: 338 |
| wwbb623 | accagtgtaaacccc | tctcttccttctgct |
| | tgggattctg | ttctcac |
| | SEQ ID NO: 339 | SEQ ID NO: 340 |
| SZ4849 | ggtagaaagtttcga | tcacacgggaacttt |
| | tcatggttgg | tctcatct |
| | SEQ ID NO: 341 | SEQ ID NO: 342 |
| LY659 | cacgatcagaaaagc | aacccatctgatgat |
| | tatctcgtctaaca | aagggtag |
| | SEQ ID NO: 343 | SEQ ID NO: 344 |
| wrl54 | gacagaagtgaaaca | gcaaagttccaagga |
| | gaaacatctgcag | aatctgatatg |
| | SEQ ID NO: 345 | SEQ ID NO: 346 |
| wwbb944 | atgcataatccaaag | ccacaactccaacaa |
| | ctgcctgaagtg | taactaaccct |
| | SEQ ID NO: 347 | SEQ ID NO: 348 |
| neww19 | gccgttgattcctgg | attgccgaaagcttt |
| | gtaggtga | aaagactccg |
| | SEQ ID NO: 349 | SEQ ID NO: 350 |
| SZ1413 | taccagatacgcata | cttacctgaaatcag |
| | acatagtggga | tttgcttcatact |
| | SEQ ID NO: 351 | SEQ ID NO: 352 |
| SZ4976 | ccatttccgggaagg | gtgtcacaatcttct |
| | tgattaattgc | atcgagat |
| | SEQ ID NO: 353 | SEQ ID NO: 354 |
| SZ2020 | atagaaaccttgaca | gggttcaacacaaaa |
| | gccaaagac | cggtcctatcatta |
| | SEQ ID NO: 355 | SEQ ID NO: 356 |
| SZ5112 | agttttgtggacgaa | ataagaaatcgtccc |
| | attgtcttttt | actcttc |
| | SEQ ID NO: 357 | SEQ ID NO: 358 |
| SZ9195 | aacaatttgcgattt | gaattgggatgagga |
| | cctgtacttgt | gataagaaa |
| | SEQ ID NO: 359 | SEQ ID NO: 360 |
| SZ2250 | taacccttgtcataa | tgttcatgttgttgt |
| | tgcagtgtttc | acagcacgc |
| | SEQ ID NO: 361 | SEQ ID NO: 362 |
| SZ2270 | attagtaagacacca | gaagcatttgtaatc |
| | atgacctcaca | agtccgacattc |
| | SEQ ID NO: 363 | SEQ ID NO: 364 |
| SZ2365 | agttggaaatccttt | tcgcacatcccatca |
| | ttcatccccttt | gtttcaatctga |
| | SEQ ID NO: 365 | SEQ ID NO: 366 |
| zwyxll88 | ctacatctgatgact | catgttttccattgg |
| | acagcttgtgcat | atctggat |
| | SEQ ID NO: 367 | SEQ ID NO: 368 |
| Yzwy320 | attcatctcgtgcct | tgaagacaagggaga |
| | atcaactt | tttaacacattaa |
| | SEQ ID NO: 369 | SEQ ID NO: 370 |
| wwbb1l39 | cgactcatccatccg | gatccaagaatttaa |
| | attataa | cgactccacaacca |
| | SEQ ID NO: 371 | SEQ ID NO: 372 |
| SZ9364 | aattgttttcataat | gcttttcgaggatcc |
| | actcctctc | acttgaattttta |
| | SEQ ID NO: 373 | SEQ ID NO: 374 |
| SZ2614 | ctagccaatagagac | gaaaatttgggagtt |
| | cgatgaacaattat | caacctgtcg |
| | SEQ ID NO: 375 | SEQ ID NO: 376 |
| TSS24702 | caaggaatatagcaa | attggcccaatttcg |
| | cgcttgaagaacaag | tggtgtttg |
| | ac | SEQ ID NO: 378 |
| | SEQ ID NO: 377 | |
| TSS248 | agtgggcctaaaaat | caaaggctgtaaaaa |
| | aaagctt | gtgtacaaaaatcat |
| | SEQ ID NO: 379 | tta |
| | | SEQ ID NO: 380 |
| SZ2733 | aacaacccctatgcc | cgaagaggaacagca |
| | atgaact | taagagtgtaccaaa |
| | SEQ ID NO: 381 | t |
| | | SEQ ID NO: 382 |
| SZ2805 | gtggaattggtggag | cagaaaagaaggtgg |
| | ctctgcataca | gaaacttactc |
| | SEQ ID NO: 383 | SEQ ID NO: 384 |
| SZ2888 | ttcggggtcgtgaga | ggtatgattaaactg |
| | atgttat | acggttccctc |
| | SEQ ID NO: 385 | SEQ ID NO: 386 |
| SZ2987 | agtaggtttggcaga | agtgagttgtttttg |
| | ttgactttgaaatcg | gaccaa |
| | | SEQ ID NO: 388 |
| | SEQ ID NO: 387 | |
| zwyxl207 | ttgtcccgtaagtca | gactggaaaacacat |
| | acagattcaaat | taaacagcatta |
| | SEQ ID NO: 389 | SEQ ID NO: 390 |
| CY1709 | ttcagtgtactgccg | gtcttaaactgaaga |
| | aattgaag | acacctctgccg |
| | SEQ ID NO: 391 | SEQ ID NO: 392 |
| SZ3067 | gcaagttaatcatgc | gttcgtccataacct |
| | agaagtcat | agctctttcttca |
| | SEQ ID NO: 393 | SEQ ID NO: 394 |
| SZ5253 | tcttgttccttggag | tatagacatggaggg |
| | attcaaagttg | acaatggtgaa |
| | SEQ ID NO: 395 | SEQ ID NO: 396 |
| Wzwy255 | tacgtcaaaacccag | cagaacccatacgct |
| | tttgatt | tcaac |
| | SEQ ID NO: 397 | SEQ ID NO: 398 |
| S981 | aggggaagcttattt | gttaagcgtttgaat |
| | tcttagggaggttg | ctgatcaactgttgt |
| | SEQ ID NO: 399 | |
| | | SEQ ID NO: 400 |
| S1005 | ggccttctcaagttc | taatgggaagctctt |
| | ttctgctgtgaagat | ggttgtatttggg |
| | t | SEQ ID NO: 402 |
| | SEQ ID NO: 401 | |
| SZ9471 | ttcttttcagttctt | acattacacaatggc |
| | aactccacacca | ttcgagaatgca |
| | SEQ ID NO: 403 | SEQ ID NO: 404 |
| dyy88 | tggaagttgatggtt | ttaactcaaattgca |
| | gaattatcat | ggatggaggtgt |
| | SEQ ID NO: 405 | SEQ ID NO: 406 |
| zwyxl080 | tttattagaagcatg | cttccgttttccttt |
| | tatcagcgg | gttgt |
| | SEQ ID NO: 407 | SEQ ID NO: 408 |
| S1202 | tagatatacaagatt | aacggctctaatgat |
| | cagcatttttt | ttgattatttgata |
| | SEQ ID NO: 409 | SEQ ID NO: 410 |
| zhwy64 | tattgctagcactcg | tgaaatgaatcgcag |
| | agagaattg | tggacaacaatag |
| | SEQ ID NO: 411 | SEQ ID NO: 412 |
| sl_1 | cccacaaagtaaagt | gaaattggagttttt |
| | ttctcccacaa | cagggtttgtgttat |
| | SEQ ID NO: 413 | |
| | | SEQ ID NO: 414 |
| XDY359 | attctatgtggtgaa | gtactgtaaaggctc |
| | agatgacaagact | aattaccttg |
| | SEQ ID NO: 415 | SEQ ID NO: 416 |
| HB15 | agttgtcctggaagt | aggtcaaatccttca |
| | catcagaccag | aaccaac |
| | SEQ ID NO: 417 | SEQ ID NO: 418 |
| SZ1609 | ccaactacattgacg | ggtttttaatcgggc |
| | attggattgttgaa | aaaggtatatt |
| | SEQ ID NO: 419 | SEQ ID NO: 420 |
| XDY354 | gtcctggctgcgtta | tccaattcatgtttt |
| | acatttttaatagg | gtggga |
| | SEQ ID NO: 421 | SEQ ID NO: 422 |
| wwl9 | ccaaacactttagat | acaagagtttgatgc |
| | atcaccctcaa | ctctacttccaacg |
| | SEQ ID NO: 423 | SEQ ID NO: 424 |
| ddyl9 | ctgcatggaaaagca | gagttggttatgttg |
| | aaattcctacaat | atgttgagttag |
| | SEQ ID NO: 425 | SEQ ID NO: 426 |
| TSS25804 | tattatcttcgactc | gatttcgttgtaagg |
| | ttcctctttgaatat | taaagggaa |
| | a | SEQ ID NO: 428 |
| | SEQ ID NO: 427 | |
| TSS249 | acaatagccagatta | catgtgtcagcttga |
| | ttaggtccgg | attttgtcttt |
| | SEQ ID NO: 429 | SEQ ID NO: 430 |
| TSS25809 | aaagatcctatagct | actagtcgatttgag |
| | tccggtcgca | ggggaaaggcta |
| | SEQ ID NO: 431 | SEQ ID NO: 432 |
| s2_6 | tagttttaacgtgca | caatgggatttctct |
| | tgcaatctc | tctccagaacaaatg |
| | SEQ ID NO: 433 | SEQ ID NO: 434 |
| TSS250 | aagtgggtcacgatt | attcttgaatccagc |
| | gaagttcttatcag | aatccaaatac |
| | SEQ ID NO: 435 | SEQ ID NO: 436 |
| S313 | tcaacctcaaccgat | caaagaagggctctc |
| | tatggccatatag | aaagactacaattct |
| | SEQ ID NO: 437 | ag |
| | | SEQ ID NO: 438 |
| SZ5388 | agaaaagtttctcga | tcatctegttttgat |
| | gggtttagtgaggga | ggttttcatg |
| | | SEQ ID NO: 440 |
| | SEQ ID NO: 439 | |
| S1252 | ggggtgatgtttatt | tctcatggcttcctc |
| | gagattgagagct | cactccaaaa |
| | SEQ ID NO: 441 | SEQ ID NO: 442 |
| XDY127 | ttaacccaaaagaag | gattcagaaccttcc |
| | agccattc | ttgect |
| | SEQ ID NO: 443 | SEQ ID NO: 444 |
| SZ9751 | aacggtaattaagcc | agagttaaaatgtgc |
| | agatatttata | actggattg |
| | SEQ ID NO: 445 | SEQ ID NO: 446 |
| wwbb1l2 | gaaatgataacatgt | attttcaaagtttga |
| | tttggaatggcgta | gggagagaacg |
| | SEQ ID NO: 447 | SEQ ID NO: 448 |
| SZ5464 | tggactattggggtt | cttatgcaacgacta |
| | tgttgatgttgt | gatctgc |
| | SEQ ID NO: 449 | SEQ ID NO: 450 |
| S321 | attattgctctttct | ccgtacctctctaaa |
| | tctttgctg | tacggaatggag |
| | SEQ ID NO: 451 | SEQ ID NO: 452 |
| XDY157 | agaattgtttcagag | tgatatgcaccattg |
| | gctaacaccat | gttgtcgga |
| | SEQ ID NO: 453 | SEQ ID NO: 454 |
| TSS25102 | tttttgtcgaagctc | attgtcaagcagcaa |
| | acaaattgtgt | acgataaat |
| | SEQ ID NO: 455 | SEQ ID NO: 456 |
| zwyx925 | aaagccaatccaact | aggtgtgaatttgtc |
| | ttacatctgaa | ctagttttcta |
| | SEQ ID NO: 457 | SEQ ID NO: 458 |
| LYY094 | gtggagaactaggtc | ctcttacatgtttta |
| | ttttcttgga | ctcttcattga |
| | SEQ ID NO: 459 | SEQ ID NO: 460 |
| zwyx555 | tacaaacttttacat | atcgcaaattacgtg |
| | gagacgctccaaata | attttaagcagtcaa |
| | ac | g |
| | SEQ ID NO: 461 | SEQ ID NO: 462 |
| XDY160 | gcattaaaaccaaag | atggtggggatgatt |
| | ccttccctgtccgaa | aagacgtaattaaa |
| | SEQ ID NO: 463 | SEQ ID NO: 464 |
| wwbb20 | ggtttctctctctcc | tgaacttaccagatc |
| | ccctatt | agcaaagagtt |
| | SEQ ID NO: 465 | SEQ ID NO: 466 |
| TSS252 | gtttggatgcttatg | ttcaggggtacaaaa |
| | gattttggag | tctagatagcaatgc |
| | SEQ ID NO: 467 | |
| | | SEQ ID NO: 468 |
| TSS253 | gatctgaagaccaag | atataatgtagttgt |
| | acaataacaatttt | tgatgattg |
| | SEQ ID NO: 469 | SEQ ID NO: 470 |
| LYY347 | ctcaacgggaagaaa | acatgcgtctaatga |
| | agggtgtttaat | agctgat |
| | SEQ ID NO: 471 | SEQ ID NO: 472 |
| SZ5769 | tttcattcgcctaac | attgtacgtggagtt |
| | tgagtttggt | tgatggacttt |
| | SEQ ID NO: 473 | SEQ ID NO: 474 |
| TSS254 | tgtgatgatcgtggg | aattgtctgcaacta |
| | catttatct | ctaaacatgacctt |
| | SEQ ID NO: 475 | SEQ ID NO: 476 |
| neww441 | aatctcctttttcct | cccttgtactggatt |
| | ggtcatcta | ccaatgacg |
| | SEQ ID NO: 477 | SEQ ID NO: 478 |
| colorl450 | cttcttcacatagcc | tgaggtacggaatta |
| | ttgtttccat | agtcatatggcacgt |
| | SEQ ID NO: 479 | |
| | | SEQ ID NO: 480 |
| SZ5929 | agctttcttggcaag | gtctgacagcttgat |
| | gaataatctt | tttgctatgaat |
| | SEQ ID NO: 481 | SEQ ID NO: 482 |
| TSS255 | gcttgcaggagggcc | tcaaaccccgacgtt |
| | ctacc | tgcatc |
| | SEQ ID NO: 483 | SEQ ID NO: 484 |
| wwbb181 | gaggaagaaaaaggg | ttcatagcctcctgc |
| | gaggaaagcc | caaatagtgc |
| | SEQ ID NO: 485 | SEQ ID NO: 486 |
| SZ6076 | ctctgttttggctag | atgcatggagaaaag |
| | tgaaacaagtaaga | ttttccct |
| | SEQ ID NO: 487 | SEQ ID NO: 488 |
| neww146 | ttgttcttgtccaaa | actttgaatggggat |
| | aatcacagcagct | tttgaggtgg |
| | SEQ ID NO: 489 | SEQ ID NO: 490 |
| SZ273 | aacatactctaaggt | ggatggatcggtttt |
| | acgctcc | ggagataa |
| | SEQ ID NO: 491 | SEQ ID NO: 492 |
| XDY231 | ctccaagccctaagg | aaaagtccacgtgtc |
| | atggtattgacc | atg |
| | SEQ ID NO: 493 | SEQ ID NO: 494 |
| SZ6268 | cattttcccgatgct | gacctttactgttac |
| | ttatgtaatca | taacccctgattta |
| | SEQ ID NO: 495 | SEQ ID NO: 496 |
| S349 | ttaatattgacgcca | acgccatcaataacg |
| | gtagaatggacca | ggtatgattcatg |
| | SEQ ID NO: 497 | SEQ ID NO: 498 |
| xccc60 | tctccctacactaca | gaagctctcgtgttt |
| | cacttttgttg | ttatcttgcatttt |
| | SEQ ID NO: 499 | SEQ ID NO: 500 |
| SZ10554 | caaaacagattagat | gcataatatacaaac |
| | cgattgggt | acttcactgc |
| | SEQ ID NO: 501 | SEQ ID NO: 502 |
| TSS260 | tatggcacaagcttg | gctgaattcatagct |
| | tatataatgtcacat | cattagtcaatac |
| | SEQ ID NO: 503 | SEQ ID NO: 504 |
| TSS261 | tgcattttcctctgt | gtatattcgacgtac |
| | gtatgtgt | aaaagatcctcca |
| | SEQ ID NO: 505 | SEQ ID NO: 506 |
| SZ6459 | ttaattaagccttat | ttgcaaacttgggat |
| | ttgctgggtg | ccatatg |
| | SEQ ID NO: 507 | SEQ ID NO: 508 |
| SZ10597 | aaagcatcgaaaaca | aaatccacgcaatat |
| | aaatcg | caaatagcgtgg |
| | SEQ ID NO: 509 | SEQ ID NO: 510 |
| TSS263 | agtgattagctagtt | ggaaggtcttgaatg |
| | tcatggcttacg | tctatgggtcttt |
| | SEQ ID NO: 511 | SEQ ID NO: 512 |
| XDY302 | actctccctatgtgg | tttgttcaccaactt |
| | tctttcaaaatt | tacaccatcc |
| | SEQ ID NO: 513 | SEQ ID NO: 514 |
| SZ6675 | caaacaagacaaaga | ggcgatggagacgga |
| | ttccattttcccaat | ggtccaa |
| | cac | SEQ ID NO: 516 |
| | SEQ ID NO: 515 | |
| wwbb244 | tacccatttgccaat | aaacccagaaggaaa |
| | cgttgatt | tttaaacaaa |
| | SEQ ID NO: 517 | SEQ ID NO: 518 |
| BDY17 | ctatgaaagtgagag | gcttaatcgaattga |
| | ctttttgt | gtcgctaaccca |
| | SEQ ID NO: 519 | SEQ ID NO: 520 |
| SZ6815 | agtagcaagcttctg | tcctctaaacatctc |
| | tgcaaaaca | cacctgattg |
| | SEQ ID NO: 521 | SEQ ID NO: 522 |
| SZ6840 | aaatgatcaaagacg | ctccaatcatcggtg |
| | tatccacaagacaac | tgaaaa |
| | ta | SEQ ID NO: 524 |
| | SEQ ID NO: 523 | |
| NCVXX77 | actgtttgggatttg | agtcagggtctgtgt |
| | tcttccacata | atgacttaaaat |
| | SEQ ID NO: 525 | SEQ ID NO: 526 |
| TSS264 | ggcttacagcgaatt | attgtttgaatttcc |
| | tcagtaacacctgt | tagacgctctcgt |
| | SEQ ID NO: 527 | SEQ ID NO: 528 |
| SZ7016 | gtttttaaatccaaa | atggggaagagagca |
| | aaggccaca | atttct |
| | SEQ ID NO: 529 | SEQ ID NO: 530 |
| SZ7070 | cttggtatacctacg | gtattgctccttgat |
| | ttcttgtat | atctgtt |
| | SEQ ID NO: 531 | SEQ ID NO: 532 |
| XDY315 | atgcaatgtgcttca | aacgactttctaagg |
| | acatcttctcactca | ttttgatgctc |
| | gg | SEQ ID NO: 534 |
| | SEQ ID NO: 533 | |
| S1234 | atcaaattaatggga | cttcagctcttgaaa |
| | gctgagtgatgac | gactccaaatg |
| | SEQ ID NO: 535 | SEQ ID NO: 536 |
| SZ11309 | tcaaaatcctaattg | ttagtggtttacact |
| | aatgcacc | ttacactacatgc |
| | SEQ ID NO: 537 | SEQ ID NO: 538 |
| GWB581 | ccacatttgataatc | ctttcagagttcatg |
| | caattcgtattt | caaaggttcttca |
| | SEQ ID NO: 539 | SEQ ID NO: 540 |
| XWB137 | cttttccattatcca | cactcatctgttctc |
| | aaccagaccaccct | atacgaagcaa |
| | SEQ ID NO: 541 | SEQ ID NO: 542 |
| XWB392 | ctctaagtaaagaga | gaacctgttttgtct |
| | atcgagctcc | gaggatt |
| | SEQ ID NO: 543 | SEQ ID NO: 544 |
| Ma | caacgaaaataaaca | ttttgattcttcagg |
| | ggtggtgatcg | ttcct |
| | SEQ ID NO: 545 | SEQ ID NO: 546 |
| SZ3450 | tttcgagcacacaaa | ttcaagggtccctaa |
| | ttttgtcaatcg | aaatcggggac |
| | SEQ ID NO: 547 | SEQ ID NO: 548 |
| S616 | ttagtaacaaattca | tattgacacgattga |
| | acgtacccctcta | ggtacgagaaga |
| | SEQ ID NO: 549 | SEQ ID NO: 550 |
| UDY1 | taaaaaaccaagaga | gacgaatttaaaatc |
| | ctgagactct | tttcactc |
| | SEQ ID NO: 551 | SEQ ID NO: 552 |
| TSS266 | gcaactgcaaaggtg | catcttctcccttgt |
| | aattcatct | tctctcccttgcc |
| | SEQ ID NO: 553 | SEQ ID NO: 554 |
| FDY190 | atctgggtagaaaac | ttagaggtagaggag |
| | gagatccga | gaggtg |
| | SEQ ID NO: 555 | SEQ ID NO: 556 |
| TSS267 | gattaggccacctga | gtggaaactagctac |
| | gtttgaatcc | ttccatct |
| | SEQ ID NO: 557 | SEQ ID NO: 558 |
| S641 | tgcaaaaccatcttc | ggttgttgctctccc |
| | taagggcttta | ttatcttgaaatt |
| | SEQ ID NO: 559 | SEQ ID NO: 560 |
| S652 | cccacatgctaatgt | acccggacaagaaaa |
| | ttgctccatcaa | tctcagata |
| | SEQ ID NO: 561 | SEQ ID NO: 562 |
| TSS268 | ttgacacaaaagata | agaggtcaataaatt |
| | ttgaggaagga | tggggacttc |
| | SEQ ID NO: 563 | SEQ ID NO: 564 |
| DYS17 | aatattgtagccatt | teggatttcatctca |
| | tegtatttteggtac | cttcagctgttcaat |
| | g | a |
| | SEQ ID NO: 565 | SEQ ID NO: 566 |
| TSS269 | cagagaggacggcga | tccaacttgcgccac |
| | aga | tttgtg |
| | SEQ ID NO: 567 | SEQ ID NO: 568 |
| S504 | aaaactgatatatgc | tttggcttgtgctcc |
| | atgagacggcttct | ttttatagta |
| | SEQ ID NO: 569 | SEQ ID NO: 570 |
| WBBBB132 | aaaactgatatatgc | tttggcttgtgctcc |
| | atgagacggcttct | ttttatagta |
| | SEQ ID NO: 571 | SEQ ID NO: 572 |
| XDY346 | ggctagacaacttaa | gcagttcatttttcc |
| | caaactccggcc | catcaaaacccatg |
| | SEQ ID NO: 573 | SEQ ID NO: 574 |
| newdy292 | gatttgtttggtttt | tgccatatttegtgt |
| | aagcaggta | ggtgtgttaattt |
| | SEQ ID NO: 575 | SEQ ID NO: 576 |
| XWB291 | ccatgcattgcctat | attgtaattttactc |
| | tccctaaattc | caatgcaatcacata |
| | SEQ ID NO: 577 | t |
| | | SEQ ID NO: 578 |
| SZ3158 | attgtagagtagtgc | taactgctaatccga |
| | caaaagtgcaaatca | aaattaagcga |
| | SEQ ID NO: 579 | SEQ ID NO: 580 |
| Y108 | tcctgttcagaacac | actagcagaagaaga |
| | attggtttttact | actagaaacctc |
| | SEQ ID NO: 581 | tcc |
| | | SEQ ID NO: 582 |
| SZ7418 | ggagattcatttgca | gagatttccttctca |
| | aagagggttg | cacttttgc |
| | SEQ ID NO: 583 | SEQ ID NO: 584 |
| TSS271 | atacaatcttttaaa | atgagacttaactca |
| | agggtgtcctgcata | gctcaactcctt |
| | SEQ ID NO: 585 | SEQ ID NO: 586 |
| XWB111 | ttttaccatttgtat | aaaccccataaccat |
| | gegcaatgagattt | aagctgcatagcca |
| | SEQ ID NO: 587 | SEQ ID NO: 588 |
| SZ7516 | tctggctccttgtaa | tgctgtgtcagagga |
| | atatgag | gcagaaaaacata |
| | SEQ ID NO: 589 | SEQ ID NO: 590 |
| S402 | ctcaatcccttgcac | taggaggagtgcaaa |
| | tggctctagc | ctttgtca |
| | SEQ ID NO: 591 | SEQ ID NO: 592 |
| SZ7624 | ttgtgtttaccacct | ttaacgatctagtca |
| | acttatgeg | accacacttcttc |
| | SEQ ID NO: 593 | SEQ ID NO: 594 |
| LY770 | ttctttctttattcg | cgagatcgaaagctc |
| | cgcacatt | agaagaatgacagca |
| | SEQ ID NO: 595 | ct |
| | | SEQ ID NO: 596 |
| S412 | ctggttcggtgagtt | aatccatctatcacc |
| | tggcaatagag | gaacacg |
| | SEQ ID NO: 597 | SEQ ID NO: 598 |
| SZ11912 | ttgggaaatatttgt | tgcaccaccatgaga |
| | tgatgtcattag | ccaattg |
| | SEQ ID NO: 599 | SEQ ID NO: 600 |
| GWB26202 | gaggccaaaaccaaa | ctatggtggcgcgtg |
| | gagaaaaatatt | gagatatcgctt |
| | SEQ ID NO: 601 | SEQ ID NO: 602 |
| LYY3327 | aggcgatcctttacg | ccagaaattcgagga |
| | tgatggctct | tgaagaagcagg |
| | SEQ ID NO: 603 | SEQ ID NO: 604 |
| S358 | gatccttttaagtac | gctgtatttgctgac |
| | ggattttcc | cgttgatgaat |
| | SEQ ID NO: 605 | SEQ ID NO: 606 |
| S369 | agttgcgaaattcta | gtagaagcttgccag |
| | caccttctaccactg | tttgtcgagg |
| | SEQ ID NO: 607 | SEQ ID NO: 608 |
| LC06 | ctacttaaagcattc | taaatcttagtgtat |
| | aggct | gctcaccat |
| | SEQ ID NO: 609 | SEQ ID NO: 610 |
| S432 | catttcagtattcct | ctcccaaacttgact |
| | gtcgtgg | ttcctcatcag |
| | SEQ ID NO: 611 | SEQ ID NO: 612 |
| XL13 | gactgtagataccaa | ctgaatcatttgatt |
| | gtgagtactgt | gttttggacaac |
| | SEQ ID NO: 613 | SEQ ID NO: 614 |
| S407 | tgactcctcggataa | ttgtgtgtggttggt |
| | ctctcactt | ttacatg |
| | SEQ ID NO: 615 | SEQ ID NO: 616 |
| nc22 | ctatgtttataacta | aactaaggtcgttta |
| | gaaccgg | gtcaaccaacgta |
| | SEQ ID NO: 617 | SEQ ID NO: 618 |
| S468 | aagccccaagttttg | gtaaataccctgcca |
| | tgaacagacac | actaggtac |
| | SEQ ID NO: 619 | SEQ ID NO: 620 |
| TSS272 | ggcgatgaagaagtt | tagaacaagcacgaa |
| | tgtccctctct | ctttgttttcga |
| | SEQ ID NO: 621 | SEQ ID NO: 622 |
| TSS274 | gacatggaagtattt | agaaatccctgtgca |
| | taatagtaaaatgct | taaagggctatata |
| | | SEQ ID NO: 624 |
| | SEQ ID NO: 623 | |
| neww27 | ctgctcctatactgg | ttgttctttgccatc |
| | caattta | ttgtcattcat |
| | SEQ ID NO: 625 | SEQ ID NO: 626 |
| neww28 | caaggggaaggaaca | tgcttgtgatgttca |
| | aaagatatta | tcagaaatgcggtgc |
| | SEQ ID NO: 627 | |
| | | SEQ ID NO: 628 |
| CHL1 | ggaagttttgaagca | gccaaggcacatcaa |
| | ataagaggcctta | tgtatttt |
| | SEQ ID NO: 629 | SEQ ID NO: 630 |
| wwbb289 | aggattcaatttgga | gctttgggttggtaa |
| | ttttcacccacgg | ttggctact |
| | SEQ ID NO: 631 | SEQ ID NO: 632 |
| LY836 | cttctgcaaaaccat | ctatatgaggatttc |
| | attgatag | agacagtctg |
| | SEQ ID NO: 633 | SEQ ID NO: 634 |
| CA201 | gcagcacaaaattca | tgggggttatattgg |
| | tcacaacaaaatcc | gggttgtgatt |
| | SEQ ID NO: 635 | SEQ ID NO: 636 |
| S602 | attgttctatgcttc | acaaatttaattttt |
| | taaccaccg | ctagcattacttatg |
| | SEQ ID NO: 637 | tttag |
| | | SEQ ID NO: 638 |

The present disclosure further provides a marker genotyping protocol, including the following steps:

1) extracting a genomic DNA of a Malus sample to be tested;

2) conducting multiplex PCR amplification on the genomic DNA sample using the primer combination to obtain an amplified product; and

3) genotyping of the amplified product by next-generation sequencing, to obtain a genotype of a Malus sample to be tested.

In the present disclosure, the genomic DNA of the Malus to be tested is extracted.

In the present disclosure, there is no special restriction on a method for extracting the genomic DNA of the Malus to be tested, and conventional methods in the field can be used.

In the present disclosure, multiplex PCR amplification is conducted on the genomic DNA of the Malus to be tested using the primer combination to obtain the amplified product.

In the present disclosure, a reaction system of the multiplex PCR amplification, calculated in 30 μL, preferably includes the following components: 8 μL of the primer combination, 8 μL of MP004_Cu Panel Mix, 50-200 ng of DNA, 10 μL of 3×T enzyme and H₂O as a balance; each primer in the primer combination has a concentration of preferably 0.24 μM; and a reaction program of the multiplex PCR amplification preferably includes: 95° C. for 3 min; 95° C. for 30 s, and 60° C. for 4 min, conducting 16 cycles; and extension at 72° C. for 4 min.

In the present disclosure, a genotype of the amplified product is measured by next-generation sequencing, to obtain a genotype of a sample of the Malus to be tested; the next-generation sequencing preferably has a depth of 1200×.

The present disclosure further provides a method for determining a trait phenotype or calculating a genomics-predicted phenotype value of a trait of the Malus sample, including the following steps:

obtaining a population average phenotype of a trait corresponding to the molecular marker as follows: fruit ripening date 159.45 DAFB, fruit cover color degree 56.35%, fruit weight 106.63 g, soluble solid content 14.85%, fruit juice pH value 3.34, fruit malate content 5.83 mg/mL, flesh firmness at harvest 12.18 kg/cm², flesh crispness at harvest 1.31 kg/cm², flesh firmness retainability 2.41 months, flesh crispness retainability 2.19 months, Fruit ring rot disease-resistance 21.34 mm, and spur tree architecture 0.99;

obtaining a genomics predicted phenotype value of a Malus sample to be tested by the method; according to the genotype of the Malus sample to be tested, and according to the population average and the genotype effect value or the genotype combination effect value, determining a genomics predicted phenotype value for a trait of the Malus sample to be tested using the following criteria or calculating a predicted phenotype value using the following prediction model:

(1) resistance to Glomerella leaf blotch:

when a genotype of S1202 is CC and a genotype of zhwy64 is CC, it is determined as disease-resistant; other genotypes are determined as susceptible;

(2) fruit shape:

when a genotype of newdy202 is CC, a genotype of SIZE2270 is GG, a genotype of SIZE5253 is CC, a genotype of SIZE9100 is GG or a genotype of SIZE9195 is AA, the fruit shape is determined to be conical-round;

when a genotype of SP031 is CC, a genotype of SP081 is not CT or a genotype of XDY231 is GG, the fruit shape is determined to be oblate-round;

(3) a chlorogenate content or a procyanidin B2 content adopts a genotype combination model;

an effect value is estimated according to a genotype combination of molecular markers for chlorogenate content or procyanidin B2 content, and a prediction model is established using a genotype combination effect value, with a formula as follows:

GPV=α×(GcE+μ)+β; where

GPV is a genomics predicted phenotype value; GcE is a genotype combination effect value of markers of the trait; μ is a mean of a phenotype of the trait in a training population; and α and β are a linear regression coefficient and a residual parameter, respectively;

(4) Fruit ripening date, soluble solid content, fruit juice pH, flesh firmness at harvest, flesh crispness at harvest, flesh firmness retainability, flesh crispness retainability, or fruit ring rot disease resistance adopts an additive model, with a formula as follows:

$$G P V = α \times ( {\sum }_{i = 1}^{k} G E + μ ) + β$$

where GPV is the genomics predicted phenotype value; GE is a genotype effect value of the marker; k is a number of markers for the trait; μ is the mean of a phenotype of the trait in the training population; and α and β are the linear regression coefficient and the residual parameter, respectively;

Fruit weight, malate content, fruit cover color degree and spur tree architecture adopt a fixed-effect model, with a prediction formula as follows:

$$G P V = α \times ( Fx + γ \times {\sum }_{i = 1}^{k} GnE + μ ) + β$$

where GPV is the genomics predicted phenotype value; Fx is a fixed genotype effect value of a fixed-effect marker; GnE is a genotype effect value of a non-fixed-effect marker of the trait; k is a number of markers for a non-fixed-effect of the trait; μ is the mean of a phenotype of the trait in a training population; γ is a shrinkage factor; and α and β are the linear regression coefficient and the residual parameter, respectively;

a fixed-effect of the fruit weight is as follows: a genotype of XDY160 is AA, or a genotype of SIZE4849 is GG or a genotype of SIZE4161 is GG, Fx is −104.8, −100.7 and −101.4, respectively;

a fixed-effect of fruit malate content is a genotype combination effect value of Ma, MA202 and SAUR-5;

a fixed-effect of fruit cover color degree is a genotype combination effect value of ZZZ162 with zwy6, and ZZZ162 with color1245;

a fixed-effect of spur tree architecture is a genotype combination effect value of neww45 with S1245, and neww45 with ww19.

The present disclosure further provides following one or more uses of the Malus trait-related molecular marker, or the genotype effect value or the genotype combination effect value, or the primer combination:

1) Trait phenotype prediction of Malus; 2) construction of fingerprint or molecular ID card of Malus; 3) genotype identification of Malus germplasm accessions; 4) hybrid breeding of Malus; and 5) molecular distinctness, uniformity and stability (DUS) test of new cultivars of Malus; wherein

the hybrid breeding of Malus comprises one or more of selection of hybrid parental materials and cross combinations, design of hybrid generation, and molecular-assisted selection of hybrid.

The technical solutions in the present disclosure will be clearly and completely described below in conjunction with the Examples of the present disclosure. Apparently, the described examples are merely some rather than all of the examples of the present disclosure. All other examples obtained by a person of ordinary skill in the art based on the examples of the present disclosure without creative efforts shall fall within the protection scope of the present disclosure.

### Example 1

1. Mining of Quantitative Trait Loci (QTL) in Whole Genome

To make the obtained QTL more versatile, segregating populations of four hybrid progenies ‘Jonathan’×‘Golden Delicious’, ‘Zisai Mingzhu’×‘Red Fuji’, ‘Zisai Mingzhu’×‘Golden Delicious’ and ‘Starkrimson’×‘Spur Fuji Miyazaki’ were selected as test materials, with a number of hybrid progeny individual plants of 1568, 1679, 2211 and 2019, respectively. ‘Jonathan’ and ‘Golden Delicious’ are one of the 7 founder cultivars of modern apple (Malus domestica Borkh.), ‘Starkrimson’ is a bud sport cultivar of another ancestor cultivar ‘Delicious’, and ‘Red Fuji’ is a direct descendant of other two ancestor cultivars ‘Ralls Janet’ and ‘Delicious’. ‘Zisai Mingzhu’ belongs to M. asiatica Nakai, and is a representative of ancient Chinese apple cultivars. From 2014 to 2020, phenotypic determination of the individual plants of the hybrid progenies above was conducted annually. Determined traits included fruit ripening date, fruit shape, fruit weight, fruit cover color degree, fruit chlorogenate content, fruit procyanidin B2 content, fruit soluble solid content, fruit juice pH value, fruit malate content, flesh firmness at harvest, flesh crispness at harvest, flesh firmness retainability, flesh crispness retainability, Glomerella leaf blotch resistance, fruit ring rot disease resistance, and spur tree architecture.

QTL mapping was conducted using phenotypic data of a single plant of the above hybrid populations for at least 3 years through MapQT and BSA-seq methods. A total of 459 QTL loci for the above fruit quality and disease resistance traits were obtained (Table 4).

TABLE 4
QTL statistics for apple fruit quality and disease resistance traits

| | | Number | Number of |
|---|---|---|---|
| Number | Trait | of | QTLs |
| 1 | Fruit weight | 3 | 72 |
| 2 | Fruit shape | 3 | 144 |
| 3 | Fruit cover color degree | 3 | 10 |
| 4 | Fruit ripening date | 3 | 54 |
| 5 | Soluble solid content | 3 | 78 |
| 6 | Sugar content (fructose or sucrose) | 3 | 5 |
| 7 | Fruit acidity (fruit malate content | 3 | 6 |
| | or juice | | |
| 8 | Chlorogenate content | 1 | 3 |
| 9 | Procyanidin B2 content | 1 | 3 |
| 10 | Flesh crispness at harvest or flesh | 1 | 24 |
| | crispness retainability | | |
| 11 | Flesh firmness at harvest or flesh | 1 | 28 |
| | firmness retainability | | |
| 12 | Glomerella leaf blotch resistance | 1 | 1 |
| 13 | Fruit ring rot disease resistance | 1 | 17 |
| 14 | Spur tree architecture | 1 | 14 |
| | Total | 4 | 459 |

2. Development of SNP Markers Based on QTL

There were many overlapped intervals in the 459 QTLs, the overlapped intervals were removed, and candidate genes were predicted near a peak of the QTL. SNP or InDel markers were developed using candidate gene coding regions, upstream regulatory sequences, or mutation loci in intergenic regions. A total of 318 SNP markers and 1 InDel marker were screened (Table 1). PCR primers were designed for the 319 markers, respectively; the amplification effect of PCR primers and the actual separation of each marker in the hybrid progenies were verified using 4 parental materials of “Jonathan”, “Golden Delicious”, “Red Fuji” and “Zisai Mingzhu” and 6-8 hybrid progenies in each of the 3 cross combinations. Determining from the actual segregation, the genotypes of the hybrid progeny of each cross combination show Mendelian segregation ratio. It is proved that the PCR primer design is successful. Sequences of the 319 labeled PCR primers are shown in Table 3.

3. Design of Multiple PCR Amplification System and Development of AppleGAP v2.0 Liquid Chip

Genomic DNA of a small tested sample was extracted, and genome was accurately quantified using Qubit®dsDNAHSAssayKit or fluorescence quantitative PCR. A 30 μL reaction system was configured using a 0.2 ml PCR tube/96-well PCR plate with the following components: 8 μL of primer combination, 8 μL of MP004_Cu Panel Mix, 50-200 ng of DNA, 10 μL of 3×T enzyme and the H₂O as the balance; each primer in the primer combination had a concentration of 0.24 μM. The PCR amplification was conducted according to the following procedures: thermal starting at 95° C. for 3 min; 95° C. for 30 s, and 60° C. for 4 min, conducting 16 cycles; and extension at 72° C. for 4 min; heat preservation at 10° C.

PCR-amplified products were purified using an AMPureXPBeads purification kit. An Illumina sequencing library was constructed using recovered PCR amplified products. A 30 μL reaction system was configured in the above PCR product purification tube with magnetic beads using the following components: 10 μL of 3×M enzyme; 1 μL of PCR primer F; 1 μL of Barcode XXR (10 μM); and 18 μL of H₂O. PCR amplification was conducted according to the following procedures: thermal starting at 95° C. for 3 min; 95° C. for 15 s, 58° C. 15 s and 72° C. 30 s, conducting 6-8 cycles; and extension at 72° C. for 4 min; heat preservation at 10° C. PCR amplified products were purified using the AMPureXPBeads purification kit.

DNA concentration of the above purified PCR products was determined, the PCR products linked to different Barcodes were mixed in equal amounts to form the AppleGAP v2.0 liquid chip, and the liquid chip was directly sequenced on the computer. Sequencing was conducted using an Illumina X10 sequencing platform and a PE150 strategy, a sequencing depth was 1000× to 1200×. Small sample test results are consistent with KASP typing of previous tests, indicating that the AppleGAP v2.0 is successfully developed.

4. Construction and Genotyping of Training Populations

A total of 1936 individual plants were selected, including random 350 hybrid progenies with more than 3 years of trait phenotypic data from 4 cross combinations (‘Jonathan’×‘Golden Delicious’, ‘Zisai Mingzhu’×‘Red Fuji’, ‘Zisai Mingzhu’×‘Golden Delicious’ and ‘Starkrimson’×‘Spur Fuji Miyazaki’), respectively, and 536 copies of Malus germplasm resources with at least 3 years of phenotypic data, to form a training population. The specific 16 phenotypic traits included fruit ripening date, fruit weight, fruit shape, fruit cover color degree, fruit soluble solid content, fruit juice pH value, fruit malate content, fruit chlorogenate content, fruit procyanidin B2 content, flesh firmness at harvest, flesh crispness at harvest, flesh firmness retainability, flesh crispness retainability, fruit ring rot disease resistance, Glomerella leaf blotch resistance, and spur tree architecture. Leaf samples of the 1936 individual plants were collected, genomic DNA was extracted, and genotyping was conducted with AppleGAP v2.0 to obtain genotype data of 319 markers of 1936 individual plants.

5. Estimation of Marker Genotype Effect Value

The marker genotype effect value was estimated using the genotype data of 319 markers and the phenotype data of 16 traits of 1936 individual plants in the above training population, with a formula as follows:

$$G E = {\sum }_{i = 1}^{m} P / m - μ$$

GE (marker genotype effect) was an effect value of a certain genotype of a certain marker;

P (individual phenotype value) was a phenotype value of a certain individual of the genotype of the marker;

m is a number of individuals with the genotype in the training population; and

μ is an average phenotype value of the trait in the training population.

The genotype effect values of the above 319 markers of the above 16 traits were estimated using the above formula, as shown in Table 2.

6. GAP Model Establishment and Testing

The above 16 traits were applicable to 5 GAP prediction models, respectively.

The above GAP models were tested by simulation selection. Selection accuracy, selection efficiency and selection progress are shown in Table 5.

TABLE 5
Technical parameters of auxiliary prediction chip AppleGAP v2.0
of apple genome

| | | | | Number | Individuals | | | |
|---|---|---|---|---|---|---|---|---|
| | Trait | Unit | Method | of | in training | Accuracy* | Selection | Exhaustiveness** |
| 1 | Fruit ripening | DAF | GAP | 52 | 1335 | 0.62 | 77.6 | 39.9 |
| 2 | Fruit shape | | MAS | 23 | 1339 | — | 34.1 | 77.2 |
| 3 | Fruit weight | g | GAP | 72 | 1450 | 0.85 | 66.1 | 68.7 |
| 4 | Fruit cover | % | GAP | 10 | 1327 | 0.56 | 65.5 | 67.9 |
| | color degree | | | | | | | |
| 5 | Soluble solid | % | GAP | 76 | 1435 | 0.40 | 62.7 | 59.0 |
| 6 | Juice pH | | GAP | 5 | 1326 | 0.64 | 71.8 | 72.1 |
| 7 | Malate | mg/m | GAP | 5 | 201 | 0.66 | 82.2 | 82.9 |
| 8 | Chlorogenate | mg/kg | GAP | 3 | 274 | 0.50 | 85.5 | 65.3 |
| | content | | | | | | | |
| 9 | Procyanidin | mg/kg | GAP | 3 | 301 | 0.95 | 65.9 | 98.2 |
| 10 | Flesh | kg/cm² | GAP | 46 | 1272 | 0.55 | 81.8 | 98.3 |
| | firmness at | | | | | | | |
| | harvet | | | | | | | |
| 11 | Flesh | kg/cm² | GAP | 46 | 1272 | 0.29 | 62.9 | 84.0 |
| | crispness at | | | | | | | |
| | harvest | | | | | | | |
| 12 | Flesh | month | GAP | 46 | 917 | 0.49 | 65.4 | 42.9 |
| | firmness | | | | | | | |
| | retainability | | | | | | | |
| 13 | Flesh crisness | month | GAP | 46 | 918 | 0.40 | 62.1 | 24.0 |
| | retainability | | | | | | | |
| 14 | Fruit ring rot | | GAP | 18 | 399 | 0.32 | 65.7 | 43.2 |
| | disease | | | | | | | |
| | resistance | | | | | | | |
| 15 | Glomerella | | MAS | 2 | 780 | — | 96.0 | 94.5 |
| | leaf blotch | | | | | | | |
| | resistance | | | | | | | |
| 16 | Spur tree | | GAP | 13 | 487 | 0.86 | 72.7 | 34.8 |
| | architecture | | | | | | | |

*Accuracy is: a linear correlation coefficient between a genomics predicted phenotype value and a measured phenotype value.
**Selection efficiency is: percentage of individuals whose genomics predicted phenotype value matches the genomics measured phenotype value among GAP-selected individuals.
***Exhaustiveness is: percentage of individuals whose genomics predicted phenotype value matches the genomics measured phenotype value among the GAP-selected individuals to the total number of individuals that meet the selection criteria in the population to be tested.

### Example 2: Hybrid Progeny Selection

In March, 2020, genomics-assisted prediction was conducted on 16,214 hybrid progenies of 13 apple cross combinations configured from 2016 to 2018 using the above AppleGAP v2.0 chip. The hybrid progenies of the batch were planted in Qianzhujiantuo Village, Beidaihe New District, Qinhuangdao City, Hebei Province. The hybrid progenies were self-rooted seedlings of 2-4 years old, and a planting density was 0.6×0.2. When sampling the leaf samples required for AppleGAP v2.0 testing, the seedling height was 2.0 m. 6 leaf discs were made using a 0.5 cm hole punch, placed in a 96-well plate, and marked; after fully filled, the 96-well plate was placed in a plastic bag pre-filled with 10 g of blue silica gel, tied and sealed. After 3 days, the leaf discs were naturally dried. The 96-well plate were taken out of the plastic bag, a soft latex cover was added, and sent to the company for DNA extraction, microarray testing and genotyping. After obtaining genotype data of all the markers, the genotype data was substituted into GAP models of each trait, and the system automatically calculated GPV values. Assisted-selection of the following traits was conducted based on GPV value. The selection criteria were as follows: the fruit ripening date was late maturity (170-185 DAFB), the fruit weight was 100-250 g, the fruit cover color degree was not less than 70%, the fruit sugar content was not less than 14.5%, the fruit malate content was 3.0-10.0 mg/mL, the fruit chlorogenate content was not more than 1.0 mg/g, the firmness and crispness of frozen flesh were maintained for more than 5 months, and there was Glomerella leaf blotch resistance. According to the above selection criteria, 77 excellent individual plants were selected from 16,214 seedlings, with a selection rate of 0.475% and a theoretical selection efficiency of 9.38%. It was expected that 7 new cultivars that meet the above selection criteria may be selected (Table 6).

TABLE 6
Actual effect of AppleGAP v2.0-selected apple hybrid progenies

| | | Selection | Selection | Selection |
|---|---|---|---|---|
| SN | Trait | criteria | ratio | efficiency |
| 1 | Fruit weight | 100~250 g | 45.5% | 61.1% |
| 2 | Fruit cover color degree | >70% | 8.04% | 63.64% |
| 3 | Fruit ripening date | 170~185 | 25.0% | 61.1% |
| | | DAFB | | |
| 4 | Fruit soluble sugar | >14.5% | 15.9% | 61.4% |
| 5 | Malate content | 3.0~10.0 | 91.0% | 100% |
| | | mg/mL | | |
| 6 | Chlorogenate content | <1.0 mg/g | 86.7% | 100% |
| | | DW | | |
| 7 | Flesh crispness retainability | ≥5.0 month | 3.48% | 64.29% |
| | | | (22.67%) | |
| 8 | Flesh firmness retainability | ≥5.0 month | 2.54% | 100% |
| | | | (100%) | |
| 9 | Glomerellaleaf blotch resistance | Resistant | 88.9% | 100% |
| | Total | | 0.475% | 9.38% |

### Example 3: Hybrid Progeny Selection

In May 2020, genomisc-assisted prediction was conducted on 3,404 hybrid progenies of 4 apple cross combinations configured in 2019 using the above AppleGAP v2.0 chip. AppleGAP v2.0 detection method and trait selection criteria were the same as in Example 1. The batch of hybrid seedlings was sown in a 32-hole plug in March 2020, and leaf samples were detected using the AppleGAP v2.0 chip in April, 2020. At that time, the seedlings consisted of 4 true leaves. One true leaf was taken with a sampling method the same as that in Example 1. According to the above selection criteria, 11 excellent individual plants were selected from 3,404 seedlings, with a selection rate of 0.323% and a theoretical selection efficiency of still 9.38%. It was expected that one new cultivar that meet the above selection criteria may be selected.

### Example 4: Hybrid Parent Selection

In February 2021, assisted-selection of hybrid parental materials was conducted using the AppleGAP v2.0. A new apple cultivar ‘Zhongnong 101’ was a new late-maturing, disease-resistant and storage-resistant cultivar bred by cross breeding in the laboratory. The parental material combination was ‘Zisai Mingzhu’×‘Red Fuji’. ‘Zhongnong 101’ matures in late October. The field incidence rate of major diseases apple rot, apple ring rot and apple early defoliation diseases of the ‘Zhongnong 101’ are significantly lower than that of main planted cultivars such as ‘Red Fuji’. But the ‘Zhongnong 101’ has fruit weight of 97 g, a relatively small fruit weight, and a hard but non-crisp flesh. It was planned to use ‘Zhongnong 101’ as one of the parental materials to configure a cross combination to select new cultivars with large fruit, red appearance, storage resistance and Glomerella leaf blotch resistance.

(1) Cultivars with large fruit shape and red appearance: genotyping was conducted on all 319 markers of apple germplasm resources using the Apple GAP v2.0. Through genotyping, an optimal cross combination was selected as ‘Zhongnong 101’×‘66-014’. ‘66-014’ is an excellent hybrid progeny of ‘Red Tsugaru’×‘Red Fuji’. The major gene markers of the coloring degree of the two cultivars are all heterozygous genotypes, such that a selection rate of the single plant of the whole red fruit of the hybrid progeny is 1/4. The parental materials are resistant to Glomerella leaf blotch, such that the hybrid progenies are all resistant to diseases. The genotypes of the major markers S2987 and XDY160 for the fruit weight of ‘Zhongnong 101’ are all heterozygous, while all the major markers for the fruit weight of ‘66-014’ are homozygous for large fruit. Therefore, a selection rate of the hybrid progeny with large fruit type is 1/4. The four main markers for the flesh crispness retainability trait are all 1:1-separated in the hybrid progeny, such that a selection rate of flesh crispness and storage durability is 1/16. In summary, the selection rate of this cross combination is 1/256, and the scale of the hybrid population can be greater than 256. This cross combination was subjected to field pollination hybridization from Apr. 21-24, 2021.

(2) Cultivars with storage durability and red fruit cover: after Apple GAP v2.0 genotyping, an optimal cross combination was selected as ‘Zhongnong 101’×‘17-199’. ‘17-199’ is a full sibling line of ‘Zhongnong 101’. The major gene markers of the coloring degree of the two cultivars are all heterozygous genotypes, such that a selection rate of the single plant of the whole red fruit of the hybrid progeny is <1/4. The parental materials are resistant to Glomerella leaf blotch, such that the hybrid progenies are all resistant to diseases. The genotypes of the major markers S2987 and XDY160 for the fruit weight of ‘Zhongnong 101’ are heterozygous, while the major markers S2987, S4161 and XDY160 for the fruit weight of ‘17-199’ are all heterozygous genotypes. Therefore, a selection rate of the hybrid progeny with large fruit type is 1/32. Three in the four main markers for the flesh crispness retainability trait are all 1:1-separated in the hybrid progeny, such that a selection rate of flesh crispness and storage durability is 1/8. In summary, the selection rate of this cross combination is 1/1024, and the scale of the hybrid population can be greater than 1024. This cross combination was subjected to field pollination hybridization from Apr. 21-24, 2021.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1) (canceled)

2) A genotype effect value or a genotype combination effect value of each genotype of a Malus trait-related molecular marker on a corresponding trait, as shown in Table 1:
TABLE 1
Genotype effect value of each genotype of each molecular marker on corresponding trait

| (1) Fruit malate content or juice pH: | | | | |
|---|---|---|---|---|
| Marker | pH | Malate | | |
| Ma | | | | |
| AA | 0.45 | −3.09 | | |
| GA | −0.06 | 0.18 | | |
| GG | −0.18 | 1.12 | | |
| MA202 | | | | |
| GG | 0.06 | −0.11 | | |
| GT | 0.03 | −1.16 | | |
| TT | −0.1 | 1.82 | | |
| mb44 | | | | |
| AA | 0.14 | −2.14 | | |
| A/insT | −0.84 | 1.22 | | |
| AT | 0.04 | −0.69 | | |
| insT | −0.94 | | | |
| TT | −0.07 | 2.12 | | |
| SAUR-05 | | | | |
| AA | −0.14 | 1.36 | | |
| GA | −0.01 | 0.07 | | |
| GG | 0.02 | −0.5 | | |
| SAUR-06 | | | | |
| AA | −0.09 | 1.78 | | |
| GA | 0 | −0.06 | | |
| GG | 0.03 | −0.52 | | |
| Genotype combination effect of Ma/MA202/SAUR-5 on fruit malate content: | | | | |
| Ma | MA202 | SAUR-5 | | |
| AA | GG | GA | −3.15 | |
| AA | GG | GG | −4.44 | |
| AA | GT | AA | −1.54 | |
| AA | GT | GA | −3.5 | |
| AA | GT | GG | −2.87 | |
| AA | TT | AA | −3.09 | |
| AA | TT | GA | −2.55 | |
| AA | TT | GG | −3.09 | |
| GA | GG | AA | | |
| GA | GG | GA | 0.11 | |
| GA | GG | GG | 0.72 | |
| GA | GT | AA | | |
| GA | GT | GA | −0.72 | |
| GA | GT | GG | −1.85 | |
| GA | TT | AA | 1.96 | |
| GA | TT | GA | 2.01 | |
| GA | TT | GG | 1.53 | |
| GG | GG | AA | | |
| GG | GG | GA | 1.86 | |
| GG | GG | GG | 0.33 | |
| GG | GT | AA | | |
| GG | GT | GA | −0.38 | |
| GG | GT | GG | 3.32 | |
| GG | TT | AA | 5.97 | |
| GG | TT | GA | 2.36 | |
| GG | TT | GG | 1 | |
| (2) Genotype combination effect of fruit chlorogenate content: | | | | |
| CA201 | S602 | CHL1 | GPV | |
| AA | AG | ins/del | 324 | |
| AA | AG | ins | 363.93 | |
| AA | GG | ins/del | 914.31 | |
| AA | GG | ins | 1041.67 | |
| AG | AG | ins/del | 467.05 | |
| AG | AG | ins | 521.6 | |
| AG | GG | ins/del | 535.2 | |
| AG | GG | ins | 855.94 | |
| (3) Genotype combination effect of fruit procyanidin B2 content: | | | | |
| S616 | S641 | S652 | GPV | |
| CC | TT | GT | 149.74 | |
| CC | TT | TT | 179.91 | |
| GC | CC | GG | 133.4 | |
| GC | CT | GG | 365.82 | |
| GC | CT | GT | 392.45 | |
| GC | CT | TT | 237.36 | |
| GC | TT | GT | 62.01 | |
| GC | TT | TT | 367.72 | |
| GG | CT | GT | 1155.12 | |
| (4) Fruit cover color degree: | | | | |
| C400 | | C1169 | | |
| AA | 1.11 | CC | 5.07 | |
| AG | −0.45 | TC | −3.48 | |
| GG | 21.65 | TT | 1.34 | |
| C1245 | | C131 | | |
| CC | 0.5 | CC | −0.46 | |
| TC | 0.42 | CT | 1.03 | |
| TT | −4.9 | | | |
| C1450 | | C318 | | |
| CC | −2.93 | CC | 43.65 | |
| TA | 3.65 | TC | 1.03 | |
| TC | 0.84 | TT | −0.53 | |
| TT | −0.01 | | | |
| C852 | | Zwy6 | | |
| TC | 1.18 | AA | 1.31 | |
| TT | −0.55 | AC | −2.58 | |
| CC | −17.88 | | | |
| Zwy29 | | Z162 | | |
| CC | 33.65 | AA | 13.11 | |
| GC | 0.4 | TA | 4.08 | |
| GG | −0.23 | TT | −16.63 | |
| Genotype combination effect of fruit cover color degree: | | | | |
| Z162 | Zwy6 | | C1245 | |
| AA | AA | 13.47 | CC | 10.53 |
| AA | AC | 12.65 | TC | 18.72 |
| AA | CC | −13.85 | TT | 2.4 |
| TA | AA | 4.65 | CC | 2.93 |
| TA | AC | 3.69 | TC | 4.98 |
| TA | CC | −15.34 | TT | 5.11 |
| TT | AA | −14.66 | CC | −17.93 |
| TT | AC | −20.82 | TC | −16.04 |
| TT | CC | −51.35 | TT | −15.76 |

(5) Fruit ring rot disease resistance:

| S1005 | | Z1080 | | Z1188 | |
|---|---|---|---|---|---|
| CC | −1.74 | CC | −5.75 | GC | −0.25 |
| CT | −1.27 | GC | −0.59 | GG | 0.11 |
| TT | 2.19 | GG | 1.11 | | |
| W2255 | | Y2320 | | S710 | |
| CC | 1.12 | GC | −0.34 | AA | 0.54 |
| CG | −1.02 | GG | 0.23 | AG | 1.47 |
| GG | 0.54 | GG | −2.33 | | |
| S733 | | S981 | | Zwy448 | |
| AA | −3.19 | CC | 3.28 | CC | −2.39 |
| GA | 2.04 | GC | 1.74 | GC | 0.72 |
| GG | −0.52 | GG | −1.05 | GG | 12.9 |
| Zwy555 | | Zwy71 | | Zwy925 | |
| AA | 0.36 | AA | 0.83 | AA | 0.32 |
| AC | −0.25 | AT | 0.51 | AG | −1.03 |
| CC | −1.91 | TT | −2.42 | GG | 6.66 |
| Hzwy486 | | S1118 | | S1122 | |
| AA | −1.02 | GG | −0.13 | CC | −1.83 |
| TA | 0.09 | TG | −0.75 | TC | 0.14 |
| TT | 0.18 | TT | 1.02 | TT | −0.15 |
| Zwy1207 | | Zwy102 | | | |
| AA | −2.61 | AA | −17.65 | | |
| CA | 2.69 | GA | 1.11 | | |
| CC | 0.75 | GG | −0.41 | | |

(6) Fruit ripening date:

| C1399 | | C1591 | | C1709 | |
|---|---|---|---|---|---|
| CC | 2.74 | CC | −3.55 | AA | −9.92 |
| TC | 2.45 | CT | 2.5 | GA | 1.27 |
| TT | −9.33 | TT | 6.65 | GG | 1.59 |
| C1821 | | C242 | | C2464 | |
| AA | 9.81 | AA | −16.45 | CC | 1.85 |
| TA | −1.19 | GA | 8.22 | CT | −0.79 |
| TT | −1.6 | GG | −5.44 | TT | 0.53 |
| C564 | | C603 | | C874 | |
| GG | 1.27 | TG | 9.8 | CC | −6.41 |
| TG | −1.35 | TT | −5.96 | CG | 10.81 |
| TT | 2.45 | | | | |
| LC06 | | LL015 | | LL1149 | |
| AA | −10.45 | CC | 1 | AA | −11.45 |
| AC | 0.82 | TC | 0.62 | GA | 9.24 |
| CC | 2.21 | TT | −1.66 | GG | −5.88 |
| LL288 | | LL531 | | LL796 | |
| AA | −9.99 | AA | 1.38 | AA | −38.83 |
| AG | −1.44 | GA | −0.71 | AG | −2.69 |
| GG | 3.14 | GG | −16.49 | GG | 4.89 |
| LL984 | | LY064 | | LY284 | |
| CC | −5.51 | CC | 5.54 | AA | 1.47 |
| CT | 8.53 | TC | −0.23 | AG | 0.45 |
| TT | −23.12 | TT | −0.58 | GG | −23.04 |
| LY591 | | LY659 | | LY770 | |
| CC | 1.44 | CC | 5.31 | CC | 0.76 |
| TC | 0.27 | CG | 0.88 | CG | −0.31 |
| TT | −2.23 | GG | −1.6 | GG | −22.57 |
| LY836 | | LYY094 | | LYY2825 | |
| CC | −46.45 | CC | −0.34 | CC | 1.61 |
| GG | −46.45 | TC | 1.57 | TC | −3.63 |
| TG | 0.86 | TT | −0.88 | | |
| TT | −0.51 | | | | |
| LYY3327 | | LYY347 | | MY154 | |
| AA | −4.44 | CC | 7.06 | AA | 4.4 |
| AT | 2.56 | GC | 2.07 | TA | 0.64 |
| TT | −7.84 | GG | −3.82 | TT | −1.05 |
| MY355 | | new146 | | new278 | |
| GG | −11.92 | AA | −0.56 | CC | −11.45 |
| GT | −1.24 | AG | −0.52 | TC | 15.55 |
| TT | 2.96 | GG | 1.22 | TT | −4.89 |
| new378 | | new441 | | S313 | |
| CC | −16.45 | AA | −10.37 | AA | −7.36 |
| TC | 8.26 | GA | −0.23 | GA | −0.59 |
| TT | −5.34 | GG | 6.2 | GG | 0.67 |
| S321 | | S349 | | | |
| AA | −0.24 | CC | −17.54 | | |
| AG | 0.79 | TC | −1.1 | | |
| GG | −5.23 | TT | 5.48 | | |
| S369 | | S358 | | S372 | |
| CC | −9.62 | AA | 4.13 | AA | 4.69 |
| CG | 9.48 | AG | −38.69 | CA | 0.16 |
| GG | −3.98 | CA | 1.34 | CC | −11.3 |
| TC | 2.7 | CC | −10.12 | | |
| TG | −1.29 | CG | −25.9 | | |
| TT | −11.97 | GG | −43.85 | | |
| S391 | | S395 | | xcc60 | |
| AA | −16.45 | CC | −0.19 | AA | 5.91 |
| AG | 8.1 | CT | −0.81 | AG | −0.51 |
| GG | −5.31 | TT | 3.47 | GG | −20.88 |
| XL13 | | XLC042 | | XLC218 | |
| CC | −10.27 | CC | −3.38 | CC | −15.15 |
| TC | 2.68 | CT | 10.37 | CT | −1.13 |
| TT | −2.45 | TT | −6.45 | TT | 11.52 |
| XLC556 | | Y108 | | Y323 | |
| AA | −14.86 | AA | −2.87 | AA | 21.05 |
| AG | −1.42 | CA | 2.51 | AC | −4.39 |
| GG | 11.1 | CC | −2.36 | CC | 1.6 |
| Y397 | | ZXX125 | | GWB1 | |
| CC | −0.22 | AA | −5.24 | GC | 14.74 |
| CT | 1.44 | AG | 8.08 | GG | −4.37 |
| TT | −2.62 | GG | −16.45 | | |
| S477 | | XWB321 | | | |
| AA | −14.65 | AA | −16.52 | | |
| AG | −1.53 | CA | −0.58 | | |
| GG | 4.22 | CC | 0.65 | | |

(7) Fruit weight:
a. Fixed effect

| | | |
|---|---|---|
| XDY160 | AA | −104.8 |
| S4849 | GG | −100.7 |
| S4161 | GG | −101.4 |

b. Non-fixed effect:

| BDY17 | | DDDD2 | | NBDY40 | |
|---|---|---|---|---|---|
| GG | 31.55 | AA | −31.7 | GG | −5.81 |
| TG | 17.13 | GA | −10.3 | GT | 7.12 |
| TT | −8.12 | GG | 8.86 | TT | −8.18 |
| newdy202 | | newdy33 | | SP011 | |
| CC | 16.69 | AA | −9.86 | CC | −5.11 |
| CT | −33.16 | TA | 0.85 | CT | 5.21 |
| TT | −102.68 | TT | 3.42 | TT | −6.59 |
| SP031 | | SP032 | | SP041 | |
| CC | −9.85 | AA | 18.06 | CC | 1.96 |
| TC | 4.85 | AC | −8.65 | CT | −1 |
| TT | 38.59 | CC | −14.94 | TT | −11.92 |
| SP042 | | SP043 | | SP044 | |
| AA | 11.03 | CC | 0.45 | AA | 13.91 |
| AT | −1.69 | CT | 3 | AT | 0.1 |
| TT | −37.88 | TT | −22.43 | TT | −7.2 |
| SP051 | | SP081 | | SP101 | |
| AA | 42.27 | AT | −94.56 | AA | 12.78 |
| GA | 6.78 | CA | −70.99 | AG | 0.59 |
| GG | −8.4 | CC | −19.98 | GG | −36.68 |
| CT | 19.3 | | | | |
| TT | −7.96 | | | | |
| XDY10 | | XDY103 | | XDY231 | |
| AA | 51.74 | AA | 61.78 | AA | 24.53 |
| AC | −101.63 | AC | 7.38 | AG | 17.27 |
| GA | −6.23 | CC | −3.14 | GG | −10.77 |
| GC | −96.44 | | | | |
| GG | −11.74 | | | | |
| XDY302 | | XDY315 | | XDY32 | |
| AA | 2.45 | AA | −58.28 | AA | −11.57 |
| AC | 5.96 | GA | 6.98 | GA | −13.36 |
| CC | −6.2 | GG | −2.13 | GG | 54.74 |
| XDY50 | | ddy6 | | dyy88 | |
| GG | −4.44 | AA | −103.2 | CC | 29.49 |
| TG | 3.89 | GA | −40.37 | TC | 37.89 |
| TT | −3.16 | GG | 18.37 | TT | −5.91 |
| FDY190 | | HB123 | | newdy292 | |
| AA | 15.23 | CC | 5.45 | CC | −104.13 |
| AT | −36.88 | TA | −103.18 | TC | −37.93 |
| TC | −9.37 | TT | 17.03 | | |
| TT | 6.96 | | | | |
| S10554 | | S10597 | | S11309 | |
| AA | −102.64 | CC | −102.64 | AA | −104.59 |
| GA | −39.72 | TC | −39.42 | TA | −36.68 |
| GG | 20.67 | TG | −103.63 | TT | 15.55 |
| TT | 20.64 | | | | |
| S11912 | | S12448 | | S12710 | |
| AA | −86.63 | AA | −102.8 | GG | −59.7 |
| GA | −37.79 | CA | −39.7 | TG | −35.66 |
| GG | 15.59 | CC | 18.18 | TT | 15.48 |
| S1348 | | S1413 | | S1609 | |
| AA | −6.15 | CC | −9.58 | AA | 0.97 |
| AT | 7.49 | CT | 13.62 | GA | −3.62 |
| TT | 29.47 | TT | 6.61 | GG | 3.72 |
| S2020 | | S2250 | | S2270 | |
| CC | 14.82 | AA | −28.59 | AA | −104.23 |
| CT | −40.42 | TA | −2.53 | GA | −38.14 |
| TT | −101.77 | TT | 2.61 | GG | 13.62 |
| S2365 | | S2614 | | S273 | |
| CC | 12.46 | AA | −80.18 | AA | 17.03 |
| CG | −36.54 | CA | −33.46 | AC | −36.99 |
| GG | −104.22 | CC | 21.78 | CC | −104.31 |
| S2733 | | S2805 | | S2888 | |
| AA | −96.57 | AA | 21.63 | GG | 16.51 |
| TA | −38.26 | AG | −38.28 | GT | −15.79 |
| TT | 18.93 | GG | −84.4 | TT | −61.16 |
| S2987 | | S3067 | | S3158 | |
| GA | −24.13 | AA | −82.09 | AA | 16.74 |
| GG | 7.91 | TA | −39.61 | AG | −37.57 |
| TT | 21.58 | GG | −104.13 | | |
| S3450 | | S3463 | | S3571 | |
| AA | −13.46 | AA | −61.89 | AA | 15.89 |
| TA | 7.66 | GA | −23.28 | AC | −37.41 |
| TT | −9.77 | GG | 9.13 | CC | −40.29 |
| S3790 | | S4161 | | S4250 | |
| CC | −102.1 | AA | 18.66 | AA | 16.02 |
| TC | −39.31 | AG | −39.18 | AG | −30.38 |
| TT | 17.47 | GG | −96.71 | GG | −51.89 |
| S4378 | | S4457 | | S4595 | |
| CC | 19.81 | CC | 19.04 | AA | 15 |
| CG | −34.4 | CT | −34.9 | AC | −11.62 |
| GG | −78.84 | TT | −70.14 | CC | −70.86 |
| CT | −88.3 | | | | |
| S4849 | | S4976 | | S5112 | |
| GG | −101.95 | AA | 14.86 | CC | 13.74 |
| TG | −41.21 | AC | −39.84 | CT | −39.1 |
| TT | 18.24 | CC | −99.67 | TT | −101.14 |
| S5253 | | S5388 | | S5464 | |
| CC | 20.05 | AA | 16.92 | AA | 21.12 |
| CT | −38.2 | AC | −36.69 | AT | −40.13 |
| TT | −97.5 | CC | −90.88 | TT | −99.31 |
| S5769 | | S5929 | | S6076 | |
| AA | 19.67 | CA | −100.45 | AA | −103.05 |
| AG | −39.38 | CC | 18.88 | CA | −40.28 |
| GG | −102.88 | CT | −38.24 | CC | 20.95 |
| TT | −102.88 | | | | |
| S6268 | | S6459 | | S6675 | |
| AT | −104.92 | AA | −88.2 | CC | 17.87 |
| GA | −103.63 | AG | −39.69 | CT | −36.9 |
| GG | 20.19 | GG | 20.51 | TT | −102.97 |
| GT | −39.65 | | | | |
| TT | −101.87 | | | | |
| S6815 | | S6840 | | S7016 | |
| CC | −103.05 | AA | 18.25 | AA | 19.12 |
| GC | −38.89 | AG | −37.43 | AG | −37.91 |
| GG | 20.16 | GG | −105.25 | GG | −105.57 |
| S7070 | | S7418 | | S7516 | |
| AA | 17.49 | AA | 16.08 | TG | −33.66 |
| AC | −36.86 | AG | −36.83 | TT | 13.28 |
| CC | −83.3 | | | | |
| S7624 | | S8111 | | S8165 | |
| AA | 1.35 | AA | −63.34 | CC | 38.37 |
| AG | −38.89 | GA | −8.31 | TC | 31.1 |
| GG | 6.65 | TT | −4.96 | | |
| S8286 | | S832 | | S8397 | |
| CC | 14.37 | CC | −28.4 | AA | −6.42 |
| CG | −37.03 | GC | 3.28 | AG | −5.81 |
| GG | 11.54 | GG | 13.76 | | |
| S8558 | | S8646 | | S8824 | |
| CC | 5.74 | CC | −48.12 | GA | −59.11 |
| TC | −4.98 | CT | 2.58 | GG | −17.84 |
| TT | −22.42 | TT | −0.16 | GT | 21.36 |
| TT | 29.89 | | | | |
| S906 | | S9100 | | S9195 | |
| CC | 21.17 | GG | 18.22 | AA | 15.1 |
| CT | 9.25 | GT | −40.89 | AG | −40.43 |
| TT | −27.6 | TT | −101.78 | GG | −102.53 |
| S9364 | | S9471 | | S9751 | |
| CC | −82.43 | AA | −5.91 | AA | 4.08 |
| TC | −35.83 | AG | 35.09 | CA | 0.25 |
| TT | 20.18 | GG | 10.76 | CC | −7.17 |
| XDY127 | | XDY157 | | XDY160 | |
| AA | −4.34 | GG | −100 | AA | −104.46 |
| AG | 31.12 | TG | −40.69 | GA | −39.4 |
| TT | 21.67 | GG | 20.13 | | |
| XDY346 | | NCVX7 | | | |
| CC | 10.22 | CC | −8.19 | | |
| CT | −27.32 | CT | 0.05 | | |
| TT | 55.47 | | | | |

(8)Fruit storability, including four traits: flesh firmness at harvest, flesh crispness at harvest, flesh
firmness retainability, and flesh crispness retainability:

| | flesh firmness | flesh crispness at | flesh firmness | flesh crispness |
|---|---|---|---|---|
| Marker | at harvest | harvest | retainability | retainability |
| GWB1 | | | | |
| GC | 1.7 | 0.14 | 1.01 | 0.8 |
| GG | −0.52 | −0.04 | −0.35 | −0.28 |
| S477 | | | | |
| AA | −1.3 | 0.02 | −0.82 | −0.58 |
| AG | −0.33 | −0.01 | −0.31 | −0.25 |
| GG | 0.56 | 0.01 | 0.39 | 0.31 |
| XWB321 | | | | |
| AA | −0.49 | 0.12 | | |
| CA | −0.42 | −0.02 | −0.28 | −0.22 |
| CC | 0.32 | 0.02 | 0.19 | 0.16 |
| GWB262 | | | | |
| AA | 0.34 | 0.04 | 0.33 | 0.27 |
| delG | −4.98 | −0.51 | 1.59 | 1.81 |
| G/delG | −2 | −0.27 | −1.74 | −1.52 |
| GA | −0.05 | −0.01 | −0.15 | |
| GG | −1.19 | −0.08 | −0.65 | |
| GWB581 | | | | |
| CC | | | | |
| TC | 0.99 | 0.09 | 0.44 | 0.3 |
| TT | −0.45 | −0.04 | −0.27 | −0.17 |
| GWB636 | | | | |
| CC | −0.33 | −0.01 | −0.32 | −0.25 |
| CT | 0.56 | 0.01 | 0.47 | 0.36 |
| TT | −0.64 | 0.19 | 0.59 | 0.81 |
| nc22 | | | | |
| AA | 1.13 | 0.16 | 0.82 | 0.77 |
| TA | 0.07 | 0.01 | 0.04 | 0 |
| TT | −0.45 | −0.06 | −0.31 | −0.24 |
| nc91 | | | | |
| cc | 0.35 | 0 | 0.32 | 0.24 |
| TC | −0.31 | −0.01 | −0.21 | −0.14 |
| TT | 0.18 | 0.09 | −0.24 | −0.25 |
| S402 | | | | |
| A/delA | 3.56 | 0.35 | | |
| AA | −1.14 | −0.14 | −0.8 | −0.64 |
| AT | −0.1 | 0 | −0.12 | −0.1 |
| delA | | | | |
| TT | 0.51 | 0.05 | 0.43 | 0.36 |
| S407 | | | | |
| CC | −0.88 | −0.07 | −0.54 | −0.41 |
| CT | 0.61 | 0.04 | 0.43 | 0.29 |
| TT | 1.66 | 0.18 | 1 | 0.9 |
| S412 | | | | |
| AA | −1.25 | −0.11 | −0.81 | −0.63 |
| AC | −0.08 | −0.01 | −0.13 | −0.11 |
| CC | 0.56 | 0.06 | 0.46 | 0.38 |
| S432 | | | | |
| AA | −1.09 | −0.07 | −0.72 | −0.58 |
| AC | −0.23 | −0.01 | −0.14 | −0.11 |
| CC | 0.68 | 0.04 | 0.43 | 0.35 |
| S468 | | | | |
| AA | 1.3 | 0.1 | 0.79 | 0.55 |
| CA | −0.45 | −0.04 | −0.31 | −0.29 |
| CC | −0.18 | −0.01 | −0.11 | −0.01 |
| S484 | | | | |
| AA | 0.28 | 0.27 | 1.59 | 2.81 |
| GA | −0.38 | −0.03 | −0.33 | −0.25 |
| GG | 0.3 | 0.02 | 0.24 | 0.18 |
| S504 | | | | |
| CC | −0.59 | −0.04 | −0.35 | −0.28 |
| CT | 1.14 | 0.09 | 0.59 | 0.48 |
| TT | 3.5 | −0.39 | | |
| WBB71 | | | | |
| AA | 0.81 | 0.04 | 0.3 | 0.23 |
| AT | −0.85 | −0.06 | −0.37 | −0.27 |
| TT | −0.61 | 0.18 | −0.78 | −0.67 |
| WBB13 | | | | |
| GC | 1.69 | 0.12 | 1.14 | 0.86 |
| GG | −0.58 | −0.04 | −0.43 | −0.32 |
| WBB132 | | | | |
| AA | 3.5 | −0.39 | | |
| TA | 1.14 | 0.09 | 0.59 | 0.48 |
| TT | −0.59 | −0.04 | −0.35 | −0.28 |
| WBB219 | | | | |
| CA | 1.57 | 0.13 | 0.84 | 0.71 |
| CC | −0.47 | −0.04 | −0.28 | −0.23 |
| WBB85 | | | | |
| CC | 0.31 | −0.01 | 0.24 | 0.18 |
| TC | −0.87 | 0 | −0.65 | −0.49 |
| TT | 0.44 | 0.05 | 0.14 | 0.15 |
| wr154 | | | | |
| AA | −0.6 | −0.05 | −0.22 | −0.22 |
| AG | 1.26 | 0.11 | 0.41 | 0.41 |
| GG | 1.28 | 0.1 | | |
| wr197 | | | | |
| AA | 1.17 | 0.01 | | |
| GA | 0.77 | 0.05 | 0.31 | 0.25 |
| GG | −0.38 | −0.02 | −0.18 | −0.14 |
| wr301 | | | | |
| AA | −1.03 | 0.31 | | |
| GA | 1.09 | 0.07 | 0.79 | 0.52 |
| GG | −0.35 | −0.02 | −0.24 | −0.15 |
| wr70 | | | | |
| CC | −1.81 | −0.19 | | |
| TC | 0.97 | 0.09 | 0.35 | 0.38 |
| TT | −0.49 | −0.04 | −0.2 | −0.21 |
| wwbb1063 | | | | |
| AA | 1.09 | 0.13 | 0.88 | 0.55 |
| GA | −0.42 | −0.04 | −0.35 | −0.26 |
| GG | −0.16 | −0.02 | −0.12 | −0.02 |
| wwbb112 | | | | |
| CC | −0.56 | −0.04 | −0.28 | −0.2 |
| CT | −0.01 | −0.02 | 0.05 | 0.03 |
| TT | 0.43 | 0.06 | 0.13 | 0.13 |
| wwbb1139 | | | | |
| AA | −1.51 | −0.15 | −0.68 | −0.45 |
| GA | −0.1 | 0.02 | −0.07 | −0.05 |
| GG | 0.88 | 0.05 | 0.35 | 0.25 |
| wwbb1159 | | | | |
| CC | −0.14 | 0 | −0.25 | −0.17 |
| CT | −0.15 | −0.01 | −0.08 | −0.02 |
| TT | 0.95 | 0.05 | 0.93 | 0.53 |
| wwbb1200 | | | | |
| CC | −0.41 | −0.02 | −0.15 | −0.16 |
| GC | 0.08 | 0 | 0.05 | 0.09 |
| GG | 0.83 | 0.08 | 0.14 | 0.08 |
| wwbb1277 | | | | |
| GG | −1.3 | 0.19 | | |
| TG | 0.55 | 0.02 | 0.53 | 0.34 |
| TT | −0.28 | −0.01 | −0.28 | −0.18 |
| wwbb1332 | | | | |
| TC | 0.62 | 0.02 | 0.52 | 0.34 |
| TT | −0.29 | −0.01 | −0.27 | −0.18 |
| wwbb1373 | | | | |
| AA | −0.36 | −0.03 | −0.26 | −0.16 |
| AG | 0.66 | 0.05 | 0.51 | 0.31 |
| GG | 0.58 | 0.1 | | |
| wwbb1441 | | | | |
| CC | 3.82 | −0.09 | | |
| TC | 0.71 | 0.04 | 0.26 | 0.26 |
| TT | −0.3 | −0.01 | −0.11 | −0.11 |
| wwbb1444 | | | | |
| AA | −7.05 | −0.84 | | |
| GA | 1.02 | 0.07 | 0.78 | 0.54 |
| GG | −0.31 | −0.02 | −0.23 | −0.16 |
| wwbb181 | | | | |
| AA | 1.29 | 0.11 | 0.81 | 0.6 |
| GA | −0.35 | 0.02 | −0.24 | −0.18 |
| GG | −0.84 | −0.09 | −0.61 | −0.44 |
| wwbb20 | | | | |
| AA | 9.14 | 0.56 | | |
| GA | 1.04 | 0.07 | 0.46 | 0.38 |
| GG | −0.64 | −0.04 | −0.31 | −0.25 |
| wwbb244 | | | | |
| CC | 0.83 | 0.08 | 0.48 | 0.43 |
| TC | 0.09 | 0 | 0.01 | 0 |
| TT | −0.73 | −0.05 | −0.32 | −0.27 |
| wwbb289 | | | | |
| GG | −0.1 | −0.01 | −0.1 | −0.08 |
| GT | 0.71 | 0.12 | 0.78 | 0.63 |
| wwbb378 | | | | |
| AA | 1.07 | 0.1 | 0.73 | 0.54 |
| GA | −0.51 | −0.04 | −0.27 | −0.2 |
| GG | −0.03 | −0.03 | −0.06 | −0.02 |
| wwbb411 | | | | |
| AA | 0.26 | 0.03 | 0.12 | 0.09 |
| AT | −0.25 | −0.02 | −0.12 | −0.09 |
| TT | 0.03 | −0.03 | 0.03 | 0.1 |
| wwbb623 | | | | |
| CC | −0.55 | −0.03 | −0.45 | −0.36 |
| CT | −0.4 | −0.06 | −0.06 | −0.09 |
| TT | 0.54 | 0.08 | 0.17 | 0.19 |
| wwbb944 | | | | |
| AA | −0.68 | −0.05 | −0.2 | −0.2 |
| AC | 1.33 | 0.11 | 0.38 | 0.39 |
| CC | 1.57 | 0.02 | | |
| XWB111 | | | | |
| AA | −0.23 | −0.01 | −0.01 | 0.03 |
| AG | 0.47 | 0.01 | 0.01 | −0.07 |
| GG | 0.43 | 0.1 | 2.59 | 2.81 |
| XWB137 | | | | |
| AA | 3.5 | −0.39 | | |
| GA | 1.02 | 0.09 | 0.44 | 0.3 |
| GG | −0.48 | −0.04 | −0.26 | −0.18 |
| XWB291 | | | | |
| CC | 1.65 | 0.19 | 1.24 | 1.12 |
| CG | | | | |
| TC | 0.03 | 0.01 | 0.1 | 0.05 |
| TG | 26.05 | 0.62 | | |
| TT | −0.65 | −0.08 | −0.62 | −0.5 |
| XWB392 | | | | |
| CC | | | | |
| TC | 1.04 | 0.1 | 0.5 | 0.33 |
| TT | −0.48 | −0.04 | −0.3 | −0.19 |

(9) Soluble solid content:

| ddy19 | | HB15 | | s1_1 | |
|---|---|---|---|---|---|
| CC | 0.06 | AA | −0.28 | CC | 0 |
| CT | −0.04 | AG | 0.17 | CT | −0.04 |
| TT | −0.42 | GG | −0.01 | TT | 0.09 |
| s2_6 | | s5_42 | | s6_75 | |
| AA | −0.05 | AA | 0.03 | CC | −0.06 |
| TA | 0.08 | GA | −0.11 | CT | 0.14 |
| TT | −0.13 | GG | 0.04 | TT | −1.65 |
| s7_27 | | s8_17 | | XDY354 | |
| GG | 0.07 | CC | −1.2 | CC | −1.7 |
| GT | −0.04 | T/insAC | −1.63 | TC | −0.23 |
| TT | −0.49 | TC | 0.04 | TT | 0.12 |
| TT | −0.05 | | | | |
| XDY359 | | f10_114 | | f2_1 | |
| AA | −0.17 | AA | −0.12 | GG | −0.36 |
| AT | 0.05 | AG | 0.08 | GT | −0.03 |
| TT | 0.06 | GG | 0.2 | TT | 0.54 |
| f4_1 | | f5_3 | | f6_25 | |
| AA | −0.29 | GG | 0.02 | CC | −0.44 |
| GA | 0.15 | GT | 0 | TC | 0.11 |
| GG | −0.12 | TT | −1.19 | TT | 0.17 |
| f8_14 | | NNEVE | | SU201 | |
| AA | 0.12 | CC | −2.96 | AA | −0.24 |
| AT | 0.11 | TC | −0.01 | TA | 0.07 |
| TT | −0.4 | TT | 0.06 | TT | 0.05 |
| SU203 | | SU204 | | XDY368 | |
| CC | 0.16 | AA | −3.84 | AA | 0.36 |
| TA | −0.63 | AG | −2.67 | AG | 0.13 |
| TC | 0.17 | CA | −2.03 | GG | −0.24 |
| TT | −0.09 | CC | −0.14 | | |
| CG | 0.32 | | | | |
| GG | 0.4 | | | | |
| DYS17 | | TSS202 | | TSS203 | |
| AA | 0.05 | CC | 0.04 | CC | 0.16 |
| AG | −0.1 | CT | −0.17 | TC | −0.03 |
| GG | −1.62 | TT | −1.76 | | |
| TSS206 | | TSS209 | | TSS210 | |
| AA | −0.14 | CC | 0.07 | AA | −0.05 |
| AG | 0.2 | CT | −0.04 | AC | 0.31 |
| GG | −0.16 | TT | −0.08 | CC | 0 |
| TSS212 | | TSS213 | | TSS214 | |
| TA | −0.22 | CC | 0.36 | CC | −0.11 |
| TT | 0.1 | CT | −0.09 | CT | 0.32 |
| TT | −0.34 | TT | −0.88 | | |
| TSS215 | | TSS216 | | TSS217 | |
| CC | 0.04 | CC | 0.02 | CC | 0.05 |
| CT | −0.01 | CT | 0.05 | TC | −0.06 |
| TT | −0.59 | TT | −0.12 | TT | 0.02 |
| TSS218 | | TSS219 | | TSS221 | |
| GG | 0.17 | CC | −2.49 | insC/insCTCC | −3.27 |
| GT | −0.15 | TC | 0.18 | insCTCC | −0.96 |
| TT | −0.63 | TT | −0.09 | T/insCTCC | 0.25 |
| TA | 4.64 | | | | |
| TT | −0.07 | | | | |
| TSS222 | | TSS223 | | TSS225 | |
| AA | 0 | CC | −0.05 | CT | |
| AG | 0.07 | TC | 0.06 | GG | 0.14 |
| GG | −0.1 | TT | 0.03 | GT | 0.01 |
| TT | −0.22 | | | | |
| TSS226 | | TSS228 | | TSS229 | |
| AA | −0.1 | CC | 0.1 | AA | −0.02 |
| AG | 0.06 | TC | 0.12 | AG | 0.01 |
| GG | −0.47 | TT | −0.16 | GG | 0.01 |
| TSS232 | | TSS233 | | TSS234 | |
| AA | 0.09 | AA | 0.19 | AA | 0.08 |
| CA | −0.2 | GA | −0.27 | delGT | |
| CC | −0.43 | GG | −0.18 | G/delGT | 0.56 |
| GA | −0.29 | | | | |
| GG | 0.07 | | | | |
| TSS235 | | TSS236 | | TSS237 | |
| AA | −0.58 | AA | −0.41 | AA | −0.04 |
| AC | 0.04 | AG | −0.14 | AG | −0.02 |
| CC | 0.09 | GG | 0.42 | GG | 0.18 |
| TSS238 | | TSS240 | | TSS241 | |
| CC | 0.09 | CC | 0.03 | AA | −0.27 |
| TC | −0.05 | TC | −0.08 | AG | 0.18 |
| TT | −0.41 | TT | 0.15 | GG | 0.09 |
| GT | 0.15 | | | | |
| TT | 0.15 | | | | |
| TSS242 | | TSS245 | | TSS247 | |
| AA | −0.02 | CC | 0 | CC | 0 |
| AG | −0.1 | CT | 0.05 | CT | 0.1 |
| GG | 0.24 | TT | −0.07 | TT | −0.65 |
| TSS248 | | TSS249 | | TSS250 | |
| GG | 0.29 | AA | −0.08 | CC | −0.15 |
| TG | 0.03 | AG | 0.01 | CG | 0.03 |
| TT | −0.13 | GG | 0.06 | GG | 0.33 |
| TSS251 | | TSS252 | | TSS253 | |
| CC | 0.32 | AA | −0.19 | AA | 0.13 |
| TC | −0.12 | GA | 0.12 | GA | 0.14 |
| TT | −0.03 | GG | −0.08 | GG | −0.2 |
| TSS254 | | TSS255 | | TSS258−4 | |
| CC | −2.85 | CC | −0.18 | AA | −0.46 |
| GG | 0.09 | CT | 0.16 | GA | 0.01 |
| GT | 0.03 | TT | 0.49 | GG | 0.02 |
| TT | −0.36 | | | | |
| TSS260 | | TSS261 | | TSS263 | |
| AA | −0.18 | CC | 0 | AA | 0.56 |
| CA | 0.05 | CT | −1.31 | CA | 0.08 |
| CC | 0.11 | GC | 0.1 | CC | −0.29 |
| | | GG | −0.05 | C/M3 | −0.03 |
| | | GT | −1.47 | C/M4 | 0.32 |
| | | | | C/N4 | −2 |
| | | | | C/N3′ | 2.2 |
| TSS264 | | TSS266 | | | |
| CC | 0.38 | AA | −0.4 | | |
| TC | 0.07 | AC | 0.06 | | |
| TT | −0.14 | CC | 0.23 | | |
| TSS267 | | TSS268 | | TSS269 | |
| AA | 0.25 | AA | 0.15 | CC | 0.42 |
| GA | −0.11 | CA | 0.17 | TC | −0.14 |
| GG | −0.54 | CC | −0.53 | TT | −0.08 |
| TSS271 | | TSS272 | | TSS274 | |
| AA | −0.1 | | | AC | 1 |
| TA | 0.23 | TC | −0.04 | CC | 0.22 |
| TT | −0.16 | TT | 0.09 | TA | −2.85 |
| TC | 0.03 | | | | |
| TT | −0.36 | | | | |
| UDY1 | | TSS258-9 | | | |
| AA | −0.63 | CC | −0.09 | | |
| GA | 0.29 | TC | 0.03 | | |
| GG | −0.43 | TT | 0.01 | | |

(10) Spur tree architecture:

| | | | | ww19 | |
|---|---|---|---|---|---|
| neww45 | | S1234 | | (neww45 ≠ CC) | |
| CC | −0.29 | TA | 0.11 | AA | 0.1 |
| TC | 0.04 | TT | −0.18 | AG | −0.09 |
| TT | 0.22 | | | | |
| ww19(S1234 = TT; | | ww19 (S1234 = TA: | | S1246 | |
| neww45 = CC) | | neww45 = CC) | | (neww45 = CC) | |
| AA | −0.17 | AA | −0.03 | C/delC | 0.04 |
| AG | 0.15 | AG | 0.03 | CC | −0.03 |
| S1246 | | S1246 | | zxx57 | |
| (neww45 = TC) | | (neww45 = TT) | | (neww45 = CC) | |
| C/delC | −0.07 | C/delC | 0.16 | del/ins | 0.12 |
| CC | 0.06 | CC | −0.14 | ins/ins | −0.13 |
| zxx57 (neww45 ≠ CC) | | | | | |
| del/ins | −0.08 | | | | |
| ins/ins | 0.09 | | | | |

wherein the Malus trait-related molecular marker comprises one or more of molecular markers in Table 2,
TABLE 2
Malus trait-re ated molecular marker

| | | | Genomic position | Ref | Alt |
|---|---|---|---|---|---|
| Marker name | Associated trait | Chromosome | (GDDH13 vl.1) | | |
| TSS202 | Soluble solid content | Chr00 | 6,746,071 | c | T |
| TSS203 | Soluble solid content | Chr00 | 12,096,767 | T | C |
| LY064 | Fruit ripening date | Chr00 | 16,403,660 | T | C |
| TSS206 | Soluble solid content | Chr00 | 34,424,368 | A | G |
| SP011 | Fruit shape | Chr01 | 6,583,636 | C | T |
| XDY10 | Fruit shape | Chr01 | 11,115,477 | G | A |
| s7_27 | Fruit fructose content | Chr01 | 22,352,268 | G | T |
| SU203 | Fruit fructose content | Chr01 | 23,318,475 | T | C |
| s8_17 | Fruit fructose content | Chr01 | 23,369,940 | T | C |
| TSS209 | Soluble solid content | Chr01 | 25,049,222 | C | T |
| f2_1 | Fruit fructose content | Chr01 | 25,310,876 | G | T |
| NNENE21 | Fruit fructose content | Chr01 | 25,501,449 | T | C |
| SZ8111 | Fruit weight | Chr01 | 26,192,298 | G | A |
| TSS210 | Soluble solid content | Chr01 | 26,623,907 | A | C |
| LL015-02 | Fruit ripening date | Chr01 | 26,658,863 | T | C |
| SU204 | Fruit fructose content | Chr01 | 27,244,389 | C | G |
| SZ8165 | Fruit weight | Chr0l | 27,290,902 | T | C |
| LY284 | Fruit ripening date | Chr0l | 27,432,954 | A | G |
| MY154 | Fruit ripening date | Chr0l | 27,556,598 | T | A |
| TSS212 | Soluble solid content | Chr0l | 29,044,153 | T | A |
| TSS213-02 | Soluble solid content | Chr0l | 29,200,389 | C | T |
| f4-1 | Fruit fructose content | Chr0l | 29,661,155 | G | A |
| TSS214 | Soluble solid content | Chr0l | 29,878,772 | C | T |
| TSS215 | Soluble solid content | Chr0l | 31,169,847 | C | T |
| f5-3 | Fruit fructose content | Chr0l | 31,183,130 | G | T |
| TSS216 | Soluble solid content | Chr02 | 2,621,710 | C | T |
| S710 | Fruit ring rot disease | Chr02 | 2,772,109 | A | G |
| | resistance-Lw048 | | | | |
| S733 | Fruit ring rot disease | Chr02 | 6,751,401 | G | A |
| | resistance-Lw048 | | | | |
| wwbb1444 | Fruit storability | Chr02 | 10,479,553 | G | A |
| SZ3463 | Fruit weight | Chr02 | 11,277,610 | G | A |
| wr301 | Fruit storability | Chr02 | 11,357,058 | G | A |
| SZ3571 | Fruit weight | Chr02 | 13,418,996 | A | C |
| TSS217 | Soluble solid content | Chr02 | 14,244,433 | T | C |
| SZ8286 | Fruit weight | Chr02 | 14,633,892 | C | G |
| zwvx71 | Fruit ring rot disease | Chr02 | 14,761,120 | A | T |
| | resistance-Lw048 | | | | |
| XDY32 | Fruit shape | Chr02 | 16,058,673 | G | A |
| ddv6 | Fruit weight | Chr02 | 16,060,933 | G | A |
| SZ3790 | Fruit weight | Chr02 | 20,662,023 | T | C |
| wwbb1063 | Fruit storability | Chr02 | 21,045,042 | G | A |
| SZ8397-02 | Fruit weight | Chr02 | 22,674,062 | A | G |
| SZ12448 | Fruit weight | Chr02 | 23,876,017 | C | A |
| SZ8558 | Fruit weight | Chr02 | 26,929,426 | T | C |
| SZ4161 | Fruit weight | Chr02 | 27,622,440 | A | G |
| f_25 | Fruit fructose content | Chr03 | 1,341,497 | T | C |
| newdv202 | Fruit shape | Chr03 | 3,150,985 | C | T |
| XDY50 | Fruit shape | Chr03 | 4,516,062 | T | G |
| f_14 | Fruit fructose content | Chr03 | 4,604,581 | A | T |
| XDY368 | Fruit fructose content | Chr03 | 4,677,137 | A | G |
| zwvx448 | Fruit ring rot disease | Chr03 | 5,372,925 | G | C |
| | resistance-Lw048 | | | | |
| SP031 | Fruit shape | Chr03 | 6,857,641 | T | C |
| SP032 | Fruit shape | Chr03 | 8,650,170 | A | C |
| NBDY40 | Fruit shape | Chr03 | 10,949,240 | G | T |
| zxxl25 | Fruit ripening date | Chr03 | 12,128,397 | A | G |
| neww378 | Fruit ripening date | Chr03 | 13,366,200 | T | C |
| S391 | Fruit ripening date | Chr03 | 15,156,984 | G | A |
| XLC042 | Fruit ripening date | Chr03 | 15,359,091 | C | T |
| LL984 | Fruit ripening date | Chr03 | 16,499,163 | C | T |
| WBBBB219 | Fruit storability | Chr03 | 17,338,844 | C | A |
| DDDD2 | Fruit shape | Chr03 | 17,759,874 | G | A |
| CY242 | Fruit ripening date | Chr03 | 17,834,913 | G | A |
| CY564 | Fruit ripening date | Chr03 | 19,114,123 | T | G |
| XDY103 | Fruit shape | Chr03 | 19,585,209 | A | C |
| S395-02 | Fruit ripening date | Chr03 | 22,021,686 | C | T |
| CY603 | Fruit ripening date | Chr03 | 23,418,983 | T | G |
| TSS218 | Soluble solid content | Chr03 | 23,915,298 | G | T |
| GWBI | Fruit ripening date + | Chr03 | 25,532,731 | G | C |
| | Fruit storabilitv | | | | |
| CY874 | Fruit ripening date | Chr03 | 26,163,955 | C | G |
| XLC218 | Fruit ripening date | Chr03 | 26,432,199 | C | T |
| WBBB71 | Fruit storabilitv | Chr03 | 26,684,680 | A | T |
| XLC556 | Fruit ripening date | Chr03 | 27,276,582 | A | G |
| nc91 | Fruit storabilitv | Chr03 | 28,907,974 | T | C |
| XWB321 | Fruit ripening date + | Chr03 | 29,398,499 | C | A |
| | Fruit storabilitv | | | | |
| LL1499 | Fruit ripening date | Chr03 | 30,229,140 | G | A |
| S372 | Fruit ripening date | Chr03 | 30,500,136 | C | A |
| neww278 | Fruit ripening date | Chr03 | 30,528,992 | T | C |
| GWB636 | Fruit storabilitv | Chr03 | 30,754,593 | C | T |
| LL288 | Fruit ripening date | Chr03 | 31,196,252 | A | G |
| WBBBB13 | Fruit storabilitv | Chr03 | 31,718,792 | G | C |
| LL531 | Fruit ripening date | Chr03 | 31,728,088 | G | A |
| WBBBB85 | Fruit storabilitv | Chr03 | 33,202,419 | T | C |
| LL796 | Fruit ripening date | Chr03 | 33,430,897 | A | G |
| S477 | Fruit ripening date + | Chr03 | 34,732,032 | A | G |
| | Fruit storability | | | | |
| MY355 | Fruit ripening date | Chr03 | 35,351,895 | G | T |
| neww45 | Spur tree architecture | Chr03 | 35,480,903 | C | T |
| S484 | Fruit storability | Chr03 | 36,414,853 | G | A |
| TSS219 | Soluble solid content | Chr04 | 6,230,752 | T | C |
| TSS221 | Soluble solid content | Chr04 | 7,779,751 | T | insCTCC |
| TSS222-02 | Soluble solid content | Chr04 | 17,134,357 | A | G |
| TSS223 | Soluble solid content | Chr04 | 18,362,465 | I | C |
| TSS225 | Soluble solid content | Chr04 | 23,107,219 | G | T |
| SP041 | Fruit shape | Chr04 | 23,306,061 | C | T |
| SP043 | Fruit shape | Chr04 | 25,208,568 | C | T |
| SP044 | Fruit shape | Chr04 | 27,497,239 | A | T |
| SP042 | Fruit shape | Chr04 | 30,693,212 | A | T |
| CY1399-02 | Fruit ripening date | Chr05 | 25,051,407 | T | C |
| CY1591 | Fruit ripening date | Chr05 | 28,648,715 | C | T |
| SP051 | Fruit shape | Chr05 | 30,658,901 | G | A |
| zwv6 | Fruit cover color degree | Chr05 | 37,652,411 | A | C |
| color1169 | Fruit cover color degree | Chr05 | 45,823,619 | T | C |
| S1118 | Fruit ring rot disease | Chr06 | 4,827,886 | T | G |
| | resistance-Zz26 | | | | |
| LYY2825 | Fruit ripening date | Chr06 | 10,902,491 | T | C |
| s542 | Fruit fructose content | Chr06 | 14,113,242 | G | A |
| SZ8646 | Fruit weight | Chr06 | 32,321,333 | C | T |
| wrl97 | Fruit storability | Chr06 | 32,387,198 | G | A |
| TSS226 | Soluble solid content | Chr07 | 23,239,054 | A | G |
| zwvv29 | Fruit cover color degree | Chr08 | 2,089,437 | G | C |
| colorl31 | Fruit cover color degree | Chr08 | 4,081,610 | C | T |
| TSS228 | Soluble solid content | Chr08 | 4,090,027 | T | C |
| color318 | Fruit cover color degree | Chr08 | 4,870,463 | T | C |
| mb44 | Fruit malate content | Chr08 | 7,674,347 | A | T |
| MA202 | Fruit malate content | Chr08 | 8,728,049 | G | T |
| TSS229 | Soluble solid content | Chr08 | 10,233,508 | A | G |
| s675 | Fruit fructose content | Chr08 | 11,373,292 | C | T |
| SAUR-05 | Fruit malate content | Chr08 | 11,648,389 | G | A |
| SAUR-06 | Fruit malate content | Chr08 | 11,648,653 | G | A |
| SP081 | Fruit shape | Chr08 | 13,916,500 | C | T |
| SZ8824 | Fruit weight | Chr09 | 921,113 | G | T |
| TSS232 | Soluble solid content | Chr09 | 18,329,880 | C | A |
| C400 | Fruit cover color | Chr09 | 19,032,403 | A | G |
| | degree | | | | |
| TSS233 | Soluble solid content | Chr09 | 19,435,642 | G | A |
| TSS234 | Soluble solid content | Chr09 | 22,432,228 | G | A |
| TSS235 | Soluble solid content | Chr09 | 24,803,629 | A | C |
| TSS236 | Soluble solid content | Chr09 | 24,807,215 | A | G |
| TSS237 | Soluble solid content | Chr09 | 25,957,418 | A | G |
| TSS238 | Soluble solid content | Chr09 | 27,916,358 | T | C |
| f10_1149 | Fruit fructose content | Chr09 | 30,376,294 | A | G |
| TSS240 | Soluble solid content | Chr09 | 30,893,803 | T | C |
| TSS241 | Soluble solid content | Chr09 | 32,254,297 | A | G |
| TSS242_ | Soluble solid content | Chr09 | 32,808,259 | A | G |
| ZZZ162 | Fruit cover color degree | Chr09 | 35,557,774 | T | A |
| TSS245 | Soluble solid content | Chr09 | 35,814,699 | C | T |
| SU201 | Fruit fructose content | Chr09 | 36,400,519 | T | A |
| color852 | Fruit cover color degree | Chr10 | 1,527,990 | T | C |
| neww26 | Spur tree architecture | Chr10 | 6,721,115 | C | T |
| wwbb1159 | Fruit storability | Chr10 | 9,403,318 | C | T |
| wwbb1277 | Fruit storability | Chr10 | 12,478,767 | T | G |
| wwbb1332 | Fruit storability | Chr10 | 19,778,489 | T | C |
| wwbb1200 | Fruit storability | Chr10 | 25,072,352 | G | C |
| wwbb1373 | Fruit storability | Chr10 | 25,583,532 | A | G |
| SI122 | Fruit ring rot disease | Chr10 | 26,131,644 | T | C |
| | resistance-Zz26 | | | | |
| Zzwv102 | Fruit ring rot disease | Chr10 | 35,049,752 | G | A |
| | resistance-Zz26 | | | | |
| Hzwv486 | Fruit ring rot disease | Chr10 | 37,766,922 | T | A |
| | resistance-Zz26 | | | | |
| SP101 | Fruit shape | Chr10 | 38,100,894 | A | G |
| wwbb378 | Fruit storability | Chr11 | 1,391,510 | G | A |
| wwbb411 | Fruit storability | Chr11 | 1,922,241 | A | T |
| HB123 | Fruit weight | Chr11 | 3,176,361 | T | C |
| newdv33 | Fruit shape | Chr11 | 4,386,624 | T | A |
| SZ1348 | Fruit weight | Chr11 | 20,016,627 | A | T |
| colorl245 | Fruit cover color degree | Chr11 | 29,379,387 | T | C |
| SZ832 | Fruit weight | Chr11 | 29,379,468 | G | C |
| SZ906 | Fruit weight | Chr11 | 32,455,108 | C | T |
| wwbb1441 | Fruit storability | Chr11 | 32,559,107 | T | C |
| Y323 | Fruit ripening date | Chr12 | 3,694,757 | A | C |
| LY591 | Fruit ripening date | Chr12 | 5,361,905 | T | c |
| SZ4250 | Fruit weight | Chr12 | 7,775,358 | A | G |
| Y397 | Fruit ripening date | Chr12 | 7,970,761 | C | T |
| SZ4378 | Fruit weight | Chr12 | 10,871,681 | C | G |
| SI246 | Spur tree architecture | Chr12 | 11,323,438 | C | A |
| zxx51 | Spur tree architecture | Chr12 | 12,132,418 | G | C |
| SZ4457 | Fruit weight | Chr12 | 12,264,258 | C | T |
| SZ4595 | Fruit weight | Chr12 | 15,224,557 | A | c |
| neww5 | Spur tree architecture | Chr12 | 16,395,680 | T | A |
| CY1821 | Fruit ripening date | Chr12 | 16,632,402 | T | A |
| SZ12710 | Fruit weight | Chr12 | 17,104,265 | T | G |
| neww61 | Spur tree architecture | Chr12 | 17,124,574 | T | G |
| wr70 | Fruit storability | Chr12 | 17,767,195 | T | C |
| neww16 | Spur tree architecture | Chr12 | 18,746,403 | G | T |
| SZ9100 | Fruit weight | Chr12 | 21,400,482 | G | T |
| CY2464 | Fruit ripening date | Chr12 | 21,676,040 | C | T |
| wwbb623 | Fruit storability | Chr12 | 22,203,610 | C | T |
| SZ4849 | Fruit weight | Chr12 | 23,710,003 | T | G |
| LY659 | Fruit ripening date | Chr12 | 24,607,594 | c | G |
| wr154 | Fruit storability | Chr12 | 24,778,590 | A | G |
| wwbb944 | Fruit storability | Chr12 | 28,128,567 | A | C |
| neww19 | Spur tree architecture | Chr12 | 28,321,850 | C | T |
| SZ1413 | Fruit weight | Chr13 | 2,101,864 | C | T |
| SZ4976 | Fruit weight | Chr13 | 3,350,768 | A | c |
| SZ2020 | Fruit weight | Chr13 | 4,926,176 | C | T |
| SZ5112 | Fruit weight | Chr13 | 6,066,422 | C | T |
| SZ9195 | Fruit weight | Chr13 | 6,537,121 | A | G |
| SZ2250 | Fruit weight | Chr13 | 8,952,017 | T | A |
| SZ2270 | Fruit weight | Chr13 | 11,870,499 | G | A |
| SZ2365 | Fruit weight | Chr13 | 13,933,420 | C | G |
| zwvx1188 | Fruit ring rot disease | Chr13 | 19,612,655 | G | C |
| | resistance-Lsl | | | | |
| Yzwv320 | Fruit ring rot disease | Chr13 | 24,655,606 | G | C |
| | resistance-Lw023 | | | | |
| wwbb1139 | Fruit storabilitv | Chr14 | 3,790,454 | G | A |
| SZ9364 | Fruit weight | Chr14 | 7,682,281 | T | C |
| SZ2614 | Fruit weight | Chr14 | 10,291,436 | C | A |
| TSS247-02 | Soluble solid content | Chr14 | 11,534,642 | c | T |
| TSS248 | Soluble solid content | Chr14 | 12,911,779 | T | G |
| SZ2733 | Fruit weight | Chr14 | 12,985,267 | T | A |
| SZ2805 | Fruit weight | Chr14 | 20,448,307 | A | G |
| SZ2888 | Fruit weight | Chr14 | 23,187,202 | G | T |
| SZ2987 | Fruit weight | Chr14 | 25,545,532 | G | A |
| zwvx1207 | Fruit ring rot disease | Chr14 | 25,700,923 | C | A |
| | resistance-Zz26 | | | | |
| CY1709 | Fruit ripening date | Chr14 | 25,857,739 | G | A |
| SZ3067 | Fruit weight | Chr14 | 27,345,446 | T | A |
| SZ5253 | Fruit weight | Chr14 | 30,020,927 | C | T |
| Wzwv255 | Fruit ring rot disease | Chr14 | 32,273,186 | C | G |
| | resistance-Lw023 | | | | |
| S981 | Fruit ring rot disease | Chr15 | 1,851,440 | G | C |
| | resistance-Lw048 | | | | |
| S1005 | Fruit ring rot disease | Chr15 | 6,167,341 | C | T |
| | resistance-Lsl | | | | |
| SZ9471 | Fruit weight | Chr15 | 6,813,832 | A | G |
| dw88 | Fruit weight | Chr15 | 6,832,683 | T | C |
| ZWVX1080 | Fruit ring rot disease | Chr15 | 7,294,095 | G | C |
| | resistance-Lsl | | | | |
| SI202 | Glomerella leaf blotch | Chr15 | 7,518,836 | G | C |
| | resistance | | | | |
| zhwv64 | Glomerella leaf blotch | Chr15 | 7,535,961 | T | C |
| | resistance | | | | |
| s1_1 | Fruit fructose content | Chr15 | 7,961,469 | C | T |
| XDY359 | Fruit fructose content | Chr15 | 8,149,862 | A | T |
| HB15 | Fruit fructose content | Chr15 | 11,982,626 | A | G |
| SZ1609 | Fruit weight | Chr15 | 13,283,951 | G | A |
| XDY354 | Fruit fructose content | Chr15 | 14,556,321 | T | C |
| wwl9 | Spur tree architecture | Chr15 | 14,791,080 | A | G |
| ddvl9 | Fruit fructose content | Chr15 | 15,628,743 | C | T |
| TSS258-04 | Soluble solid content | Chr15 | 15,630,626 | G | A |
| TSS249 | Soluble solid content | Chr15 | 15,632,755 | A | G |
| TSS258-09 | Soluble solid content | Chr15 | 15,633,545 | T | C |
| s2_6 | Fruit fructose content | Chr15 | 16,053,147 | T | A |
| TSS250 | Soluble solid content | Chr15 | 16,541,855 | C | G |
| S313 | Fruit ripening date | Chr15 | 16,567,767 | G | A |
| SZ5388 | Fruit weight | Chr15 | 18,184,482 | A | C |
| S1252 | Spur tree architecture | Chr15 | 18,281,416 | C | A |
| XDY127 | Fruit weight | Chr15 | 18,810,707 | A | G |
| SZ9751 | Fruit weight | Chr15 | 20,670,273 | C | A |
| wwbb112 | Fruit storability | Chr15 | 20,994,595 | C | T |
| SZ5464 | Fruit weight | Chr15 | 22,313,813 | A | T |
| S321 | Fruit ripening date | Chr15 | 24,229,527 | A | G |
| XDY157 | Fruit weight | Chr15 | 24,269,269 | T | G |
| TSS251-02 | Soluble solid content | Chr15 | 24,616,358 | T | C |
| zwvx925 | Fruit ring rot disease | Chr15 | 25,553,244 | A | G |
| | resistance-Lw048 | | | | |
| LYY094 | Fruit ripening date | Chr15 | 25,641,444 | T | C |
| zwvx555 | Fruit ring rot disease | Chr15 | 25,773,261 | A | C |
| | resistance-Lw048 | | | | |
| XDY160 | Fruit weight | Chr15 | 26,425,887 | G | A |
| wwbb20 | Fruit storability | Chr15 | 26,869,888 | G | A |
| TSS252 | Soluble solid content | Chr15 | 26,963,022 | G | A |
| TSS253 | Soluble solid content | Chr15 | 26,994,265 | G | A |
| LYY347 | Fruit ripening date | Chr15 | 27,007,585 | G | C |
| SZ5769 | Fruit weight | Chr15 | 27,186,324 | A | G |
| TSS254 | Soluble solid content | Chr15 | 30,285,932 | G | T |
| neww441 | Fruit ripening date | Chr15 | 30,869,138 | G | A |
| colorl450 | Fruit cover color degree | Chr15 | 31,183,115 | T | C |
| SZ5929 | Fruit weight | Chr15 | 31,402,325 | C | T |
| TSS255 | Soluble solid content | Chr15 | 31,521,236 | C | T |
| wwbb181 | Fruit storability | Chr15 | 31,983,275 | G | A |
| SZ6076 | Fruit weight | Chr15 | 33,640,614 | C | A |
| neww146 | Fruit ripening date | Chr15 | 34,629,451 | A | G |
| SZ273 | Fruit weight | Chr15 | 37,109,478 | A | C |
| XDY231 | Fruit shape | Chr15 | 37,110,278 | A | G |
| SZ6268 | Fruit weight | Chr15 | 38,111,629 | G | T |
| S349 | Fruit ripening date | Chr15 | 38,480,628 | T | C |
| xccc60 | Fruit ripening date | Chr15 | 40,445,622 | A | G |
| SZ10554 | Fruit weight | Chr15 | 40,559,644 | G | A |
| TSS260 | Soluble solid content | Chr15 | 40,795,162 | C | A |
| TSS261 | Soluble solid content | Chr15 | 41,354,666 | G | C |
| SZ6459 | Fruit weight | Chr15 | 42,126,761 | G | A |
| SZ10597 | Fruit weight | Chr15 | 44,293,907 | T | C |
| TSS263 | Soluble solid content | Chr15 | 44,777,216 | C | A |
| XDY302 | Fruit shape | Chr15 | 44,788,654 | A | C |
| SZ6675 | Fruit weight | Chr15 | 46,848,583 | c | T |
| wwbb244 | Fruit storability | Chr15 | 47,522,379 | T | C |
| BDY17 | Fruit shape | Chr15 | 47,794,257 | T | G |
| SZ6815 | Fruit weight | Chr15 | 48,048,672 | G | C |
| SZ6840 | Fruit weight | Chr15 | 50,117,476 | A | G |
| NCVXX77 | Fruit shape | Chr15 | 50,136,492 | C | T |
| TSS264 | Soluble solid content | Chr15 | 50,153,850 | T | C |
| SZ7016 | Fruit weight | Chr15 | 51,632,414 | A | G |
| SZ7070 | Fruit weight | Chr15 | 53,318,871 | A | C |
| XDY315 | Fruit shape | Chr15 | 53,446,034 | G | A |
| S1234 | Spur tree architecture | Chr16 | 509,607 | A | T |
| SZ11309 | Fruit weight | Chr16 | 574,182 | T | A |
| GWB581 | Fruit storability | Chr16 | 1,440,735 | T | C |
| XWB137 | Fruit storability | Chr16 | 1,540,780 | G | A |
| XWB392 | Fruit storability | Chr16 | 3,059,129 | T | C |
| Ma | Fruit malate content | Chr16 | 3,179,027 | G | A |
| SZ3450 | Fruit weight | Chr16 | 3,299,638 | T | A |
| S616 | Fruit procvanidin B2 | Chr16 | 3,404,957 | C | G |
| | content | | | | |
| UDY1 | Soluble solid content | Chr16 | 3,441,632 | G | A |
| TSS266 | Soluble solid content | Chr16 | 3,452,478 | A | C |
| FDY190 | Fruit weight | Chr16 | 3,552,434 | A | T |
| TSS267 | Soluble solid content | Chr16 | 3,554,158 | G | A |
| S641 | Fruit procvanidin B2 | Chr16 | 3,804,010 | T | C |
| | content | | | | |
| S652 | Fruit procvanidin B2 | Chr16 | 3,887,901 | T | G |
| | content | | | | |
| TSS268 | Soluble solid content | Chr16 | 4,273,438 | C | A |
| DYS17 | Soluble solid content | Chr16 | 5,309,087 | A | G |
| TSS269 | Soluble solid content | Chr16 | 5,583,186 | T | C |
| S504 | Fruit storability | Chr16 | 5,979,221 | C | T |
| WBBBB132 | Fruit storability | Chr16 | 5,979,292 | T | A |
| XDY346 | Fruit weight | Chr16 | 6,334,724 | C | T |
| newdv292 | Fruit weight | Chr16 | 6,398,049 | T | C |
| XWB291 | Fruit storability | Chr16 | 9,127,269 | T | C |
| SZ3158 | Fruit weight | Chr16 | 9,187,936 | A | G |
| Y108 | Fruit ripening date | Chr16 | 9,422,810 | C | A |
| SZ7418 | Fruit weight | Chr16 | 9,428,581 | A | G |
| TSS271 | Soluble solid content | Chr16 | 9,470,752 | T | A |
| XWB111 | Fruit storability | Chr16 | 10,070,698 | A | G |
| SZ7516 | Fruit weight | Chr16 | 10,928,020 | T | G |
| S402 | Fruit storability | Chr16 | 13,200,599 | A | T |
| SZ7624 | Fruit weight | Chr16 | 13,203,946 | A | G |
| LY770 | Fruit ripening date | Chr16 | 13,243,241 | C | G |
| S412 | Fruit storability | Chr16 | 13,579,418 | A | C |
| SZ11912 | Fruit weight | Chr16 | 13,933,577 | G | A |
| GWB262-02 | Fruit storability | Chr16 | 14,398,932 | G | A |
| LYY3327 | Fruit ripening date | Chr16 | 14,729,859 | A | T |
| S358 | Fruit ripening date | Chr16 | 16,542,947 | C | A |
| S369 | Fruit ripening date | Chr16 | 18,318,696 | T | G |
| LC06 | Fruit ripening date | Chr16 | 20,598,023 | A | C |
| S432 | Fruit storability | Chr16 | 20,996,010 | A | C |
| XL13 | Fruit ripening date | Chr16 | 22,704,725 | T | C |
| S407 | Fruit storability | Chr16 | 23,366,479 | C | T |
| nc22 | Fruit storability | Chr16 | 38,573,461 | T | A |
| S468 | Fruit storability | Chr16 | 38,573,599 | C | A |
| TSS272 | Soluble solid content | Chr16 | 38,914,076 | T | C |
| TSS274 | Soluble solid content | Chr17 | 5,996,233 | T | C |
| neww27 | Spur tree architecture | Chr17 | 7,843,681 | G | T |
| neww28 | Spur tree architecture | Chr17 | 9,275,707 | T | C |
| CHL1 | Fruit chlorogenate | Chr17 | 15,953,079 | TACTG | del |
| | content | | | CCCAA | |
| wwbb289 | Fruit storability | Chr17 | 21,947,295 | G | T |
| LY836 | Fruit ripening date | Chr17 | 22,524,548 | T | G |
| CA201 | Fruit chlorogenate | Chr17 | 23,199,602 | A | G |
| | content | | | | |
| S602 | Fruit chlorogenate | Chr17 | 27,353,986 | G | A |
| | content | | | | |

3) (canceled)

4) A marker genotyping protocol, comprising the following steps:
1) extracting a genomic DNA of a Malus sample to be tested;
2) conducting multiplex polymerase chain reaction (PCR) amplification on the genomic DNA sample using a primer combination to obtain an amplified product; and
3) genotyping of the amplified product by next-generation sequencing, to obtain a genotype of a Malus sample to be tested; wherein
the primer combination comprises a primer combination shown in SEQ ID NO. 1 to SEQ ID NO. 638, and is used for marker PCR amplification when marker genotyping.

5) The method according to claim 4, wherein a reaction system of the multiplex PCR amplification in step 2), calculated in 30 μL, comprises the following components: 8 μL of the primer combination, 8 μL of MP004_Cu Panel Mix, 50-200 ng of DNA, 10 μL of 3×T enzyme and H₂O as a balance; each primer in the primer combination has a concentration of 0.24 μM; and a reaction program of the multiplex PCR amplification comprises: 95° C. for 3 min; 95° C. for 30 s, and 60° C. for 4 min, conducting 16 cycles; and extension at 72° C. for 4 min.

6) The method according to claim 4, wherein the next-generation sequencing in step 3) has a depth of 1200×.

7) A method for determining a trait phenotype or calculating a genomics-predicted phenotype value of a trait of the Malus sample, comprising the following steps:
obtaining a population average phenotype of a trait corresponding to a molecular marker as follows: fruit ripening date 159.45 DAFB, fruit cover color degree 56.35%, fruit weight 106.63 g, soluble solid content 14.85%, fruit juice pH value 3.34, fruit malate content 5.83 mg/mL, flesh firmness at harvest 12.18 kg/cm², flesh crispness at harvest 1.31 kg/cm², flesh firmness retainability 2.41 months, flesh crispness retainability 2.19 months, fruit ring rot disease resistance 21.34 mm, and spur tree architecture 0.99;
obtaining a genomics predicted phenotype value of a Malus sample to be tested; according to the genotype of the Malus sample to be tested, and according to the population average and the genotype effect value or the genotype combination effect value according to claim 2, determining a genomics predicted phenotype value for a trait of the Malus sample to be tested using the following criteria or calculating a predicted phenotype value using the following prediction model; wherein
the standard comprises:
(1) resistance to Glomerella leaf blotch:
when a genotype of S1202 is CC and a genotype of zhwy64 is CC, it is determined as disease-resistant; other genotypes are determined as susceptible;
(2) fruit shape:
when a genotype of newdy202 is CC, a genotype of SIZE2270 is GG, a genotype of SIZE5253 is CC, a genotype of SIZE9100 is GG or a genotype of SIZE9195 is AA, the fruit shape is determined to be conical-round;
when a genotype of SP031 is CC, a genotype of SP081 is not CT or a genotype of XDY231 is GG, the fruit shape is determined to be oblate-round;
the prediction model comprises:
(3) a chlorogenate content or a procyanidin B2 content adopts a genotype combination model;
an effect value is estimated according to a genotype combination of molecular markers for chlorogenate or procyanidin B2 content, and a prediction model is established using a genotype combination effect value, with a formula as follows:
GPV=α×(GcE+μ)+β; wherein
GPV is a genomics predicted phenotype value; GcE is a genotype combination effect value of markers of the trait; μ is a mean of a phenotype of the trait in a training population; and α and β are a linear regression coefficient and a residual parameter, respectively;
(4) fruit ripening date, soluble solid content, fruit juice pH, flesh firmness at harvest, flesh crispness at harvest, flesh firmness retainability, flesh crispness retainability, or fruit ring rot disease resistance adopts an additive model, with a formula as follows:
$$G P V = α \times ( {\sum }_{i = 1}^{k} G E + μ ) + β$$
wherein GPV is the genomics predicted phenotype value; GE is a genotype effect value of the marker; k is a number of markers for the trait; μ is the mean of a phenotype of the trait in the training population; and α and β are the linear regression coefficient and the residual parameter, respectively;
fruit weight, malate content, fruit cover color degree and spur tree architecture adopt a fixed-effect model, with a prediction formula as follows:
$$G P V = α \times ( Fx + γ \times {\sum }_{i = 1}^{k} GnE + μ ) + β$$
wherein GPV is the genomics predicted phenotype value; Fx is a fixed genotype effect value of a fixed-effect marker; GnE is a genotype effect value of a non-fixed-effect marker of the trait; k is a number of markers for a non-fixed-effect of the trait; μ is the mean of a phenotype of the trait in a training population; γ is a shrinkage factor; and α and β are the linear regression coefficient and the residual parameter, respectively;
a fixed-effect of the fruit weight is as follows: a genotype of XDY160 is AA, or a genotype of SIZE4849 is GG or a genotype of SIZE4161 is GG, Fx is −104.8, −100.7 and −101.4, respectively;
a fixed-effect of fruit malate content is a genotype combination effect value of Ma, MA202 and SAUR-5;
a fixed-effect of fruit cover color degree is a genotype combination effect value of ZZZ162 with zwy6, and ZZZ162 with color1245;
a fixed-effect of spur tree architecture is a genotype combination effect value of neww45 with S1245, and neww45 with ww19.

8) (canceled)


SEQUENCE LISTING (638 sequences)

SEQ ID NO: 1 - DNA, 25 bp, Artificial Sequence
  upstream primer of TSS202
agataaatgactcggcaagttcaaa

SEQ ID NO: 2 - DNA, 25 bp, Artificial Sequence
  downstream primer of TSS202
tgtctccagattgcgaaagataatc

SEQ ID NO: 3 - DNA, 20 bp, Artificial Sequence
  upstream primer of TSS203
tgccatcttccggttgaatt

SEQ ID NO: 4 - DNA, 24 bp, Artificial Sequence
  downstream primer of TSS203
ccgatagagaatgtcttgctacgt

SEQ ID NO: 5 - DNA, 26 bp, Artificial Sequence
  upstream primer of LY064
attttagctcttcccaaaagtacgac

SEQ ID NO: 6 - DNA, 26 bp, Artificial Sequence
  downstream primer of LY064
tgtgcgcaatgtttgaattttgaagt

SEQ ID NO: 7 - DNA, 28 bp, Artificial Sequence
  upstream primer of TSS206
tgtccactctttcgttccaaaaatgaag

SEQ ID NO: 8 - DNA, 33 bp, Artificial Sequence
  downstream primer of TSS206
tgtagctaatctctaaactttaaagtcgattat

SEQ ID NO: 9 - DNA, 18 bp, Artificial Sequence
  upstream primer of SP011
gaaggggcacggcacaac

SEQ ID NO: 10 - DNA, 23 bp, Artificial Sequence
  downstream primer of SP011
actgaccttggaccatggggcct

SEQ ID NO: 11 - DNA, 24 bp, Artificial Sequence
  upstream primer of XDY10
tgaagtagctctttcatcaaaccc

SEQ ID NO: 12 - DNA, 26 bp, Artificial Sequence
  downstream primer of XDY10
aggaaagttgatgtggactcgaatga

SEQ ID NO: 13 - DNA, 21 bp, Artificial Sequence
  upstream primer of s7_27
tcgaaatttgttgagtcattg

SEQ ID NO: 14 - DNA, 27 bp, Artificial Sequence
  downstream primer of s7_27
ggtcagtcttttctgtacatctgcagg

SEQ ID NO: 15 - DNA, 27 bp, Artificial Sequence
  upstream primer of SU203
ccttgagacacaaccctataccaactg

SEQ ID NO: 16 - DNA, 25 bp, Artificial Sequence
  downstream primer of SU203
tgagagtggatatctagggtttcgt

SEQ ID NO: 17 - DNA, 28 bp, Artificial Sequence
  upstream primer of s8_17
caaagtgtattaagtttgtgtcagcact

SEQ ID NO: 18 - DNA, 23 bp, Artificial Sequence
  downstream primer of s8_17
gaaggaaatcgagggaaaataga

SEQ ID NO: 19 - DNA, 32 bp, Artificial Sequence
  upstream primer of TSS209
gtgcaaatttataaaaatattgatattgatag

SEQ ID NO: 20 - DNA, 31 bp, Artificial Sequence
  downstream primer of TSS209
acaggaagcaacgaaaagaatccaattcaac

SEQ ID NO: 21 - DNA, 26 bp, Artificial Sequence
  upstream primer of f2_1
ataaatgataaaaaccccggcaaatc

SEQ ID NO: 22 - DNA, 25 bp, Artificial Sequence
  downstream primer of f2_1
attaggtgatctgattcggtggatg

SEQ ID NO: 23 - DNA, 28 bp, Artificial Sequence
  upstream primer of NNENE21
gtaagcgtgcacaacttcaattaacaat

SEQ ID NO: 24 - DNA, 26 bp, Artificial Sequence
  downstream primer of NNENE21
aagggtgaacgagtataattagcaac

SEQ ID NO: 25 - DNA, 27 bp, Artificial Sequence
  upstream primer of SZ8111
tcgtaaaaattttgccaacccaagcaa

SEQ ID NO: 26 - DNA, 24 bp, Artificial Sequence
  downstream primer of SZ8111
caaaaagaccaagaagcccaaaca

SEQ ID NO: 27 - DNA, 26 bp, Artificial Sequence
  upstream primer of TSS210
ttgccttgcttccaaatattgaacat

SEQ ID NO: 28 - DNA, 26 bp, Artificial Sequence
  downstream primer of TSS210
tctacccaagttaccttgccaatagc

SEQ ID NO: 29 - DNA, 21 bp, Artificial Sequence
  upstream primer of LL01502
cttttcctgctctccttcaca

SEQ ID NO: 30 - DNA, 29 bp, Artificial Sequence
  downstream primer of LL01502
tgatcttgtcccttgtcgttgatataccg

SEQ ID NO: 31 - DNA, 29 bp, Artificial Sequence
  upstream primer of SU204
cagtaaaacgacatggtccaattgatgca

SEQ ID NO: 32 - DNA, 27 bp, Artificial Sequence
  downstream primer of SU204
attcatgcatcgtcttgttcttgttag

SEQ ID NO: 33 - DNA, 26 bp, Artificial Sequence
  upstream primer of SZ8165
agcgtacatggcttttatggtatgag

SEQ ID NO: 34 - DNA, 24 bp, Artificial Sequence
  downstream primer of SZ8165
tcaattcccactctccaaaagttt

SEQ ID NO: 35 - DNA, 21 bp, Artificial Sequence
  upstream primer of LY284
gaagcaaaacctgtcccataa

SEQ ID NO: 36 - DNA, 22 bp, Artificial Sequence
  downstream primer of LY284
tggactgagatttttcaagcca

SEQ ID NO: 37 - DNA, 25 bp, Artificial Sequence
  upstream primer of MY154
attaatgccatgcacataatttcac

SEQ ID NO: 38 - DNA, 24 bp, Artificial Sequence
  downstream primer of MY154
caaatagctaacaagaacagtggt

SEQ ID NO: 39 - DNA, 22 bp, Artificial Sequence
  upstream primer of TSS212
gagcccaaactgttattgcaaa

SEQ ID NO: 40 - DNA, 25 bp, Artificial Sequence
  downstream primer of TSS212
attcctcaattggtttgtctgcgtt

SEQ ID NO: 41 - DNA, 31 bp, Artificial Sequence
  upstream primer of TSS21302
attgagtgtcaatgaaccaaagatatacttg

SEQ ID NO: 42 - DNA, 21 bp, Artificial Sequence
  downstream primer of TSS21302
tcagggggtcaaacgacaatt

SEQ ID NO: 43 - DNA, 22 bp, Artificial Sequence
  upstream primer of f41
agacagcaaacgactaaaagag

SEQ ID NO: 44 - DNA, 28 bp, Artificial Sequence
  downstream primer of f41
tctgcaagaacatcttcacagttctgaa

SEQ ID NO: 45 - DNA, 24 bp, Artificial Sequence
  upstream primer of TSS214
tgcctagtcttgaagcagtccatg

SEQ ID NO: 46 - DNA, 28 bp, Artificial Sequence
  downstream primer of TSS214
atatgaggatcctttaatttgtggtgcc

SEQ ID NO: 47 - DNA, 22 bp, Artificial Sequence
  upstream primer of TSS215
gttctaataaaaggggtggtcc

SEQ ID NO: 48 - DNA, 29 bp, Artificial Sequence
  downstream primer of TSS215
gggcctgatgatattatgggttttgattg

SEQ ID NO: 49 - DNA, 28 bp, Artificial Sequence
  upstream primer of f5_3
ttttatttaagtgacgggtacagctcag

SEQ ID NO: 50 - DNA, 26 bp, Artificial Sequence
  downstream primer of f5_3
ctctctcctctatctcaaacccaaag

SEQ ID NO: 51 - DNA, 26 bp, Artificial Sequence
  upstream primer of TSS216
gtagtaaagttgcagtctttaagtga

SEQ ID NO: 52 - DNA, 28 bp, Artificial Sequence
  downstream primer of TSS216
acattccttcactaattcctccctaaat

SEQ ID NO: 53 - DNA, 24 bp, Artificial Sequence
  upstream primer of S710
ataaagtaattggtggaacctcca

SEQ ID NO: 54 - DNA, 27 bp, Artificial Sequence
  downstream primer of S710
aaacatcaatcctacattccattcacg

SEQ ID NO: 55 - DNA, 21 bp, Artificial Sequence
  upstream primer of S733
accaaaacgagtcaaagttca

SEQ ID NO: 56 - DNA, 21 bp, Artificial Sequence
  downstream primer of S733
attttccaagtcgtctcgttt

SEQ ID NO: 57 - DNA, 22 bp, Artificial Sequence
  upstream primer of wwbb1444
tttcctggatctgctggatttt

SEQ ID NO: 58 - DNA, 21 bp, Artificial Sequence
  downstream primer of wwbb1444
cgaggtaagaggatatacttg

SEQ ID NO: 59 - DNA, 27 bp, Artificial Sequence
  upstream primer of SZ3463
gtggatgaggccatgactctaagagaa

SEQ ID NO: 60 - DNA, 26 bp, Artificial Sequence
  downstream primer of SZ3463
ctcccaaaagttagggcaagataagg

SEQ ID NO: 61 - DNA, 23 bp, Artificial Sequence
  upstream primer of wr301
cttgactttttccttatcgtctt

SEQ ID NO: 62 - DNA, 28 bp, Artificial Sequence
  downstream primer of wr301
ggaagcggctctgaagactagtattagg

SEQ ID NO: 63 - DNA, 31 bp, Artificial Sequence
  upstream primer of SZ3571
caaacgactttagtctttcaatagttgagtt

SEQ ID NO: 64 - DNA, 23 bp, Artificial Sequence
  downstream primer of SZ3571
acatcatggagagtatttcctcc

SEQ ID NO: 65 - DNA, 22 bp, Artificial Sequence
  upstream primer of TSS217
ctaatatagctggatttaatgt

SEQ ID NO: 66 - DNA, 23 bp, Artificial Sequence
  downstream primer of TSS217
accaaatggatgaattttggagt

SEQ ID NO: 67 - DNA, 25 bp, Artificial Sequence
  upstream primer of SZ8286
ccgaacggagaaattcttcataagg

SEQ ID NO: 68 - DNA, 25 bp, Artificial Sequence
  downstream primer of SZ8286
ttaaagttgttcaagagtttctcgg

SEQ ID NO: 69 - DNA, 29 bp, Artificial Sequence
  upstream primer of zwyx71
tgaaatcatgctatatttagatgtacttt

SEQ ID NO: 70 - DNA, 26 bp, Artificial Sequence
  downstream primer of zwyx71
aactactaactgaggagaatgtgcgc

SEQ ID NO: 71 - DNA, 31 bp, Artificial Sequence
  upstream primer of XDY32
gcttcagttcatagaggagacgaccaggaac

SEQ ID NO: 72 - DNA, 21 bp, Artificial Sequence
  downstream primer of XDY32
aaactcagagatgggatgaga

SEQ ID NO: 73 - DNA, 27 bp, Artificial Sequence
  upstream primer of ddy6
gaagaagtacgagcttgtcataacaac

SEQ ID NO: 74 - DNA, 24 bp, Artificial Sequence
  downstream primer of ddy6
caacaacactgaggcattgtcaat

SEQ ID NO: 75 - DNA, 24 bp, Artificial Sequence
  upstream primer of SZ3790
gcataagtgatttaatccaccctc

SEQ ID NO: 76 - DNA, 28 bp, Artificial Sequence
  downstream primer of SZ3790
cattggaaatgtggtgcttatgagtaat

SEQ ID NO: 77 - DNA, 27 bp, Artificial Sequence
  upstream primer of wwbb1063
gaagatgaggcatgcttaaaagattcc

SEQ ID NO: 78 - DNA, 28 bp, Artificial Sequence
  downstream primer of wwbb1063
tgggggaccgataagtctttagattaat

SEQ ID NO: 79 - DNA, 24 bp, Artificial Sequence
  upstream primer of SZ839702
tttcgatcatgaaggttaggaagg

SEQ ID NO: 80 - DNA, 24 bp, Artificial Sequence
  downstream primer of SZ839702
cgaaagcagaggcactttatatcc

SEQ ID NO: 81 - DNA, 30 bp, Artificial Sequence
  upstream primer of SZ12448
attcacgcttcatctctttcctaagatagg

SEQ ID NO: 82 - DNA, 30 bp, Artificial Sequence
  downstream primer of SZ12448
gaaaaggggcatgcaaaaatcattttcctt

SEQ ID NO: 83 - DNA, 22 bp, Artificial Sequence
  upstream primer of SZ8558
aatcttgagtgatgggaagagg

SEQ ID NO: 84 - DNA, 24 bp, Artificial Sequence
  downstream primer of SZ8558
gataaagggctgtagatccttcca

SEQ ID NO: 85 - DNA, 26 bp, Artificial Sequence
  upstream primer of SZ4161
cgtttgttgaagctgagtataggggt

SEQ ID NO: 86 - DNA, 29 bp, Artificial Sequence
  downstream primer of SZ4161
catgctttgttcgatcatgttgtaccaaa

SEQ ID NO: 87 - DNA, 25 bp, Artificial Sequence
  upstream primer of f6_25
tgatgaaatgagaggaacatggctg

SEQ ID NO: 88 - DNA, 27 bp, Artificial Sequence
  downstream primer of f6_25
tcgaacttgtccctacagttcagagca

SEQ ID NO: 89 - DNA, 33 bp, Artificial Sequence
  upstream primer of newdy202
gccaaatttaaaatttagcaagttttgggctcg

SEQ ID NO: 90 - DNA, 33 bp, Artificial Sequence
  downstream primer of newdy202
aaaggcacttacaaacaagtcataaaacaaatg

SEQ ID NO: 91 - DNA, 22 bp, Artificial Sequence
  upstream primer of XDY50
ccgtacatttcccagttattag

SEQ ID NO: 92 - DNA, 28 bp, Artificial Sequence
  downstream primer of XDY50
tgtccagtctctctgtaatactctggaa

SEQ ID NO: 93 - DNA, 25 bp, Artificial Sequence
  upstream primer of f8_14
tagattaaggagctgacagagttcg

SEQ ID NO: 94 - DNA, 29 bp, Artificial Sequence
  downstream primer of f8_14
agtagttttcaacctatccatcaccggtt

SEQ ID NO: 95 - DNA, 25 bp, Artificial Sequence
  upstream primer of XDY368
tgaaaagattcaccaaatcccagcc

SEQ ID NO: 96 - DNA, 32 bp, Artificial Sequence
  downstream primer of XDY368
gctcagtttgctcttggagtacttgatggcgc

SEQ ID NO: 97 - DNA, 24 bp, Artificial Sequence
  upstream primer of zwyx448
ttctcccatactgatcatgtgact

SEQ ID NO: 98 - DNA, 26 bp, Artificial Sequence
  downstream primer of zwyx448
attgccaaagctattattggtaatga

SEQ ID NO: 99 - DNA, 27 bp, Artificial Sequence
  upstream primer of SP031
gctgaaattctaactctgtttgacatc

SEQ ID NO: 100 - DNA, 22 bp, Artificial Sequence
  downstream primer of SP031
cagtgtcaagctatggcaaatg

SEQ ID NO: 101 - DNA, 27 bp, Artificial Sequence
  upstream primer of SP032
aattggttcaaaatcctcaaaggcctt

SEQ ID NO: 102 - DNA, 27 bp, Artificial Sequence
  downstream primer of SP032
tttttctttgataaaatctatggcagg

SEQ ID NO: 103 - DNA, 26 bp, Artificial Sequence
  upstream primer of NBDY40
ccgaggtgctacttgattgttgcaaa

SEQ ID NO: 104 - DNA, 25 bp, Artificial Sequence
  downstream primer of NBDY40
ttgtaccaatgtcctacaaccgtgg

SEQ ID NO: 105 - DNA, 26 bp, Artificial Sequence
  upstream primer of zxx125
aagaagtagagggagagagagagcca

SEQ ID NO: 106 - DNA, 21 bp, Artificial Sequence
  downstream primer of zxx125
ttttctgggtgttttaatgcg

SEQ ID NO: 107 - DNA, 24 bp, Artificial Sequence
  upstream primer of neww378
attacgttcttgatatgcggattt

SEQ ID NO: 108 - DNA, 23 bp, Artificial Sequence
  downstream primer of neww378
gcaagatgagttttaatgaaccc

SEQ ID NO: 109 - DNA, 25 bp, Artificial Sequence
  upstream primer of S391
gaagcatcattatcacacgaatcag

SEQ ID NO: 110 - DNA, 25 bp, Artificial Sequence
  downstream primer of S391
atatagaaccacaggaggaatgttg

SEQ ID NO: 111 - DNA, 26 bp, Artificial Sequence
  upstream primer of XLC042
ataaatattaaatagtctaaggggtg

SEQ ID NO: 112 - DNA, 31 bp, Artificial Sequence
  downstream primer of XLC042
ccctcactcgttcccttcttttcttttcttt

SEQ ID NO: 113 - DNA, 29 bp, Artificial Sequence
  upstream primer of LL984
aatgatgtttgtcttcataatcaccccca

SEQ ID NO: 114 - DNA, 26 bp, Artificial Sequence
  downstream primer of LL984
atcgtgacttgggaaacaattctttc

SEQ ID NO: 115 - DNA, 30 bp, Artificial Sequence
  upstream primer of WBBBB219
aaaacataacgaggtgattatggggtcgta

SEQ ID NO: 116 - DNA, 24 bp, Artificial Sequence
  downstream primer of WBBBB219
tgacatagtggcaaataagaggtt

SEQ ID NO: 117 - DNA, 26 bp, Artificial Sequence
  upstream primer of DDDD2
ctagaacgtctagaaaaccaggatcc

SEQ ID NO: 118 - DNA, 29 bp, Artificial Sequence
  downstream primer of DDDD2
agatcgacaagtattcttcgcttacaact

SEQ ID NO: 119 - DNA, 27 bp, Artificial Sequence
  upstream primer of CY242
tccaaaatttgcttcataagacacagt

SEQ ID NO: 120 - DNA, 26 bp, Artificial Sequence
  downstream primer of CY242
tttcgacatttggatttgaaagttta

SEQ ID NO: 121 - DNA, 21 bp, Artificial Sequence
  upstream primer of CY564
ttcaaacagaggtaacctgag

SEQ ID NO: 122 - DNA, 25 bp, Artificial Sequence
  downstream primer of CY564
agtagaggtgtttaaggtgagatgc

SEQ ID NO: 123 - DNA, 30 bp, Artificial Sequence
  upstream primer of XDY103
tctttccattggtttgacactgatagattg

SEQ ID NO: 124 - DNA, 30 bp, Artificial Sequence
  downstream primer of XDY103
ctataacgccatttcttcttctgtgttggt

SEQ ID NO: 125 - DNA, 29 bp, Artificial Sequence
  upstream primer of S39502
tctttgacgagaatcgaatctaagacctc

SEQ ID NO: 126 - DNA, 26 bp, Artificial Sequence
  downstream primer of S39502
catgggtaatgttttgttttgagtgc

SEQ ID NO: 127 - DNA, 26 bp, Artificial Sequence
  upstream primer of CY603
ttaaacccaagaccaagcataagtct

SEQ ID NO: 128 - DNA, 25 bp, Artificial Sequence
  downstream primer of CY603
ctcatagctgcatatttcgtcgttg

SEQ ID NO: 129 - DNA, 22 bp, Artificial Sequence
  upstream primer of TSS218
acctcaaaatttgtctggttga

SEQ ID NO: 130 - DNA, 30 bp, Artificial Sequence
  downstream primer of TSS218
aaatcccagatcagaatttttcccaaaaaa

SEQ ID NO: 131 - DNA, 27 bp, Artificial Sequence
  upstream primer of GWB1
aatacaccgctcttgaataatttcccc

SEQ ID NO: 132 - DNA, 32 bp, Artificial Sequence
  downstream primer of GWB1
agatcactctaccaggtacgacaaatttccaa

SEQ ID NO: 133 - DNA, 31 bp, Artificial Sequence
  upstream primer of CY874
aagggtcaatttgttaaatcttatcatgaca

SEQ ID NO: 134 - DNA, 30 bp, Artificial Sequence
  downstream primer of CY874
gcgttaattatttgttgcttatttcaattt

SEQ ID NO: 135 - DNA, 28 bp, Artificial Sequence
  upstream primer of XLC218
tttgatgacgatgaaaacttctgggatc

SEQ ID NO: 136 - DNA, 28 bp, Artificial Sequence
  downstream primer of XLC218
ttggatcaaactaagagaatgcagcaaa

SEQ ID NO: 137 - DNA, 28 bp, Artificial Sequence
  upstream primer of WBBB71
tttgaaaactgctgtcagattcttgtga

SEQ ID NO: 138 - DNA, 26 bp, Artificial Sequence
  downstream primer of WBBB71
gaagttgaacatggtgtggtttgaaa

SEQ ID NO: 139 - DNA, 29 bp, Artificial Sequence
  upstream primer of XLC556
tagaggaaaccatcttcgtctttgttttc

SEQ ID NO: 140 - DNA, 31 bp, Artificial Sequence
  downstream primer of XLC556
aggtatctggttcctgctgatttgactgttg

SEQ ID NO: 141 - DNA, 26 bp, Artificial Sequence
  upstream primer of nc91
tgccattttcagagtgcaatatgcta

SEQ ID NO: 142 - DNA, 29 bp, Artificial Sequence
  downstream primer of nc91
tccatagcattattacggtttgtgcttga

SEQ ID NO: 143 - DNA, 24 bp, Artificial Sequence
  upstream primer of XWB321
aaaatgaggatatattcacgcaca

SEQ ID NO: 144 - DNA, 24 bp, Artificial Sequence
  downstream primer of XWB321
ctcatcccagatatgacatggcat

SEQ ID NO: 145 - DNA, 27 bp, Artificial Sequence
  upstream primer of LL1499
tggccacaaaggaaaaatatccacata

SEQ ID NO: 146 - DNA, 24 bp, Artificial Sequence
  downstream primer of LL1499
cctccagcatggttattaacaaga

SEQ ID NO: 147 - DNA, 26 bp, Artificial Sequence
  upstream primer of S372
gaatctggaccaacactgatagtcaa

SEQ ID NO: 148 - DNA, 21 bp, Artificial Sequence
  downstream primer of S372
aggctctgaataatctcgttt

SEQ ID NO: 149 - DNA, 25 bp, Artificial Sequence
  upstream primer of neww278
gcgatatgttgacaatttctccaat

SEQ ID NO: 150 - DNA, 27 bp, Artificial Sequence
  downstream primer of neww278
ggtgcacatatcaaagaatctgctaca

SEQ ID NO: 151 - DNA, 29 bp, Artificial Sequence
  upstream primer of GWB636
gtgtatactctatgtgtaatttctcctta

SEQ ID NO: 152 - DNA, 21 bp, Artificial Sequence
  downstream primer of GWB636
taagaataatcctatgttgca

SEQ ID NO: 153 - DNA, 25 bp, Artificial Sequence
  upstream primer of LL288
tggaagtttgtcgatatgcctttgt

SEQ ID NO: 154 - DNA, 23 bp, Artificial Sequence
  downstream primer of LL288
caacgtgtgtgatatttgagtct

SEQ ID NO: 155 - DNA, 21 bp, Artificial Sequence
  upstream primer of WBBBB13
ctcgttggatgtcggattctg

SEQ ID NO: 156 - DNA, 25 bp, Artificial Sequence
  downstream primer of WBBBB13
gctttaacactctctcctcgctata

SEQ ID NO: 157 - DNA, 29 bp, Artificial Sequence
  upstream primer of LL531
ctttctgggtaggaataacacgatttgtt

SEQ ID NO: 158 - DNA, 23 bp, Artificial Sequence
  downstream primer of LL531
gagaccaaactcttttctcttgt

SEQ ID NO: 159 - DNA, 23 bp, Artificial Sequence
  upstream primer of WBBBB85
tttataagacgaagaccccttga

SEQ ID NO: 160 - DNA, 21 bp, Artificial Sequence
  downstream primer of WBBBB85
aaagtcaacatcttgttcgcg

SEQ ID NO: 161 - DNA, 27 bp, Artificial Sequence
  upstream primer of LL796
gttccagtgacatcaagcataagtctc

SEQ ID NO: 162 - DNA, 24 bp, Artificial Sequence
  downstream primer of LL796
aaattgaaggatgtccatctttac

SEQ ID NO: 163 - DNA, 25 bp, Artificial Sequence
  upstream primer of S477
tttgggaattattcgttgtttcgca

SEQ ID NO: 164 - DNA, 25 bp, Artificial Sequence
  downstream primer of S477
caccgtacttggttatgtccagtat

SEQ ID NO: 165 - DNA, 32 bp, Artificial Sequence
  upstream primer of MY355
ttgatcctctactctcgaagaccaagatcatc

SEQ ID NO: 166 - DNA, 24 bp, Artificial Sequence
  downstream primer of MY355
aaagttgaaagaggatgctggccc

SEQ ID NO: 167 - DNA, 19 bp, Artificial Sequence
  upstream primer of neww45
cattctgttgggtttccgc

SEQ ID NO: 168 - DNA, 23 bp, Artificial Sequence
  downstream primer of neww45
gagaccgactcaacaatggtagt

SEQ ID NO: 169 - DNA, 26 bp, Artificial Sequence
  upstream primer of S484
gttatgtgatgtattatttcacgtgt

SEQ ID NO: 170 - DNA, 28 bp, Artificial Sequence
  downstream primer of S484
ctaacacacttggtattattatttcagt

SEQ ID NO: 171 - DNA, 21 bp, Artificial Sequence
  upstream primer of TSS219
aattctctgtttgattgccat

SEQ ID NO: 172 - DNA, 24 bp, Artificial Sequence
  downstream primer of TSS219
tttttgtaggcatttgatcgggtt

SEQ ID NO: 173 - DNA, 26 bp, Artificial Sequence
  upstream primer of TSS221
tcgtaagcaacacggcgaaaagatat

SEQ ID NO: 174 - DNA, 28 bp, Artificial Sequence
  downstream primer of TSS221
agaagctgatgaatctcagtgtttctga

SEQ ID NO: 175 - DNA, 25 bp, Artificial Sequence
  upstream primer of TSS22202
tccttcggaaagagagagaaacgag

SEQ ID NO: 176 - DNA, 30 bp, Artificial Sequence
  downstream primer of TSS22202
atcgaataaataccaactcccacttggaat

SEQ ID NO: 177 - DNA, 30 bp, Artificial Sequence
  upstream primer of TSS223
atgaccaataaatctagaacaagagtagca

SEQ ID NO: 178 - DNA, 26 bp, Artificial Sequence
  downstream primer of TSS223
atctgcactgtacacgtgtatacatc

SEQ ID NO: 179 - DNA, 28 bp, Artificial Sequence
  upstream primer of TSS225
tgcacatttccactaaccgaatatgtat

SEQ ID NO: 180 - DNA, 24 bp, Artificial Sequence
  downstream primer of TSS225
aattaaactactgtatgtgcgcct

SEQ ID NO: 181 - DNA, 30 bp, Artificial Sequence
  upstream primer of SP041
ccagtcatactgactgtagaggaacctcgt

SEQ ID NO: 182 - DNA, 21 bp, Artificial Sequence
  downstream primer of SP041
ttgtaaatttcaattctcacc

SEQ ID NO: 183 - DNA, 26 bp, Artificial Sequence
  upstream primer of SP043
tcaaggcacgattcctaattggattt

SEQ ID NO: 184 - DNA, 25 bp, Artificial Sequence
  downstream primer of SP043
ccctgaagttgatttgagtgaaggt

SEQ ID NO: 185 - DNA, 23 bp, Artificial Sequence
  upstream primer of SP044
gccaaatttatgccacatggcaa

SEQ ID NO: 186 - DNA, 22 bp, Artificial Sequence
  downstream primer of SP044
ggttcgtacagaaatgggagca

SEQ ID NO: 187 - DNA, 23 bp, Artificial Sequence
  upstream primer of SP042
atgtttatggcattgctgataca

SEQ ID NO: 188 - DNA, 26 bp, Artificial Sequence
  downstream primer of SP042
gtcgaagtgaacatgtaatagtgcca

SEQ ID NO: 189 - DNA, 28 bp, Artificial Sequence
  upstream primer of CY139902
gagtcacacctaccgaactgttaggtag

SEQ ID NO: 190 - DNA, 22 bp, Artificial Sequence
  downstream primer of CY139902
ttgaagtaagggtagattgtgt

SEQ ID NO: 191 - DNA, 26 bp, Artificial Sequence
  upstream primer of CY1591
atttccataaacttcacaggcaatga

SEQ ID NO: 192 - DNA, 30 bp, Artificial Sequence
  downstream primer of CY1591
cttctaaacccggcaaactaagttctattg

SEQ ID NO: 193 - DNA, 27 bp, Artificial Sequence
  upstream primer of SP051
taaccatgttgaaattctcgccctaca

SEQ ID NO: 194 - DNA, 25 bp, Artificial Sequence
  downstream primer of SP051
gatgtaacccttcttgagctgttca

SEQ ID NO: 195 - DNA, 23 bp, Artificial Sequence
  upstream primer of zwy6
aacgagatatcgacatgatttgt

SEQ ID NO: 196 - DNA, 21 bp, Artificial Sequence
  downstream primer of zwy6
catgcatcaagaagaagctca

SEQ ID NO: 197 - DNA, 27 bp, Artificial Sequence
  upstream primer of color1169
tgatgatgtcaaaacaacacgcttaac

SEQ ID NO: 198 - DNA, 25 bp, Artificial Sequence
  downstream primer of color1169
aaatgttgcattctgtagagctcat

SEQ ID NO: 199 - DNA, 23 bp, Artificial Sequence
  upstream primer of S1118
ctcatatcctttcaatgtccagc

SEQ ID NO: 200 - DNA, 28 bp, Artificial Sequence
  downstream primer of S1118
tacataacctcctgtcctgattgttcag

SEQ ID NO: 201 - DNA, 27 bp, Artificial Sequence
  upstream primer of LYY2825
caagagcaattaacgccatcctaattt

SEQ ID NO: 202 - DNA, 26 bp, Artificial Sequence
  downstream primer of LYY2825
cgagtggtgtttttgtttgattcttg

SEQ ID NO: 203 - DNA, 24 bp, Artificial Sequence
  upstream primer of s5_42
aaatggccacatcttcaatttgca

SEQ ID NO: 204 - DNA, 29 bp, Artificial Sequence
  downstream primer of s5_42
tccagacatattttgagtgtcacttgtca

SEQ ID NO: 205 - DNA, 22 bp, Artificial Sequence
  upstream primer of SZ8646
atgcagacataaggggaacaag

SEQ ID NO: 206 - DNA, 27 bp, Artificial Sequence
  downstream primer of SZ8646
ccagaatacagtgggaacttctcaacg

SEQ ID NO: 207 - DNA, 24 bp, Artificial Sequence
  upstream primer of wr197
tggtttgctcactaaattggatgg

SEQ ID NO: 208 - DNA, 29 bp, Artificial Sequence
  downstream primer of wr197
gcacaaatcattttcgcgaagatgacaca

SEQ ID NO: 209 - DNA, 26 bp, Artificial Sequence
  upstream primer of TSS226
atcacaacatatccaattttgcattt

SEQ ID NO: 210 - DNA, 23 bp, Artificial Sequence
  downstream primer of TSS226
ggatggttcttgtcatttaagct

SEQ ID NO: 211 - DNA, 22 bp, Artificial Sequence
  upstream primer of zwyy29
aaaacagttcgaaggttggtct

SEQ ID NO: 212 - DNA, 23 bp, Artificial Sequence
  downstream primer of zwyy29
agtcttggaaacgaagtacatct

SEQ ID NO: 213 - DNA, 28 bp, Artificial Sequence
  upstream primer of color131
cagtttcaatataccaaaggcagcagta

SEQ ID NO: 214 - DNA, 25 bp, Artificial Sequence
  downstream primer of color131
cttttgtgaatccaagtaaatgggg

SEQ ID NO: 215 - DNA, 30 bp, Artificial Sequence
  upstream primer of TSS228
ggaatacaacttttccaaccccaagtgcac

SEQ ID NO: 216 - DNA, 28 bp, Artificial Sequence
  downstream primer of TSS228
cacccttttcctttatgcttcatagaac

SEQ ID NO: 217 - DNA, 29 bp, Artificial Sequence
  upstream primer of color318
cagcaaactcatgttcttcaagtagctct

SEQ ID NO: 218 - DNA, 29 bp, Artificial Sequence
  downstream primer of color318
tggagctgtcttgtggcctaattaatcac

SEQ ID NO: 219 - DNA, 26 bp, Artificial Sequence
  upstream primer of mb44
ctcgttaaactttgttggataactct

SEQ ID NO: 220 - DNA, 30 bp, Artificial Sequence
  downstream primer of mb44
ttttggtcaaagtcatccgtttgcttacgc

SEQ ID NO: 221 - DNA, 26 bp, Artificial Sequence
  upstream primer of MA202
atggttgcatatgaagaagcaactct

SEQ ID NO: 222 - DNA, 25 bp, Artificial Sequence
  downstream primer of MA202
caagttcttcaacatatcaagccca

SEQ ID NO: 223 - DNA, 28 bp, Artificial Sequence
  upstream primer of TSS229
cttggaaatcccagttgaggttgaaggg

SEQ ID NO: 224 - DNA, 31 bp, Artificial Sequence
  downstream primer of TSS229
ttatatagcttagagggctttcttacgggct

SEQ ID NO: 225 - DNA, 21 bp, Artificial Sequence
  upstream primer of s6_75
accccatttttgttgtggacc

SEQ ID NO: 226 - DNA, 28 bp, Artificial Sequence
  downstream primer of s6_75
ggtgaggttagtgtggtcgaattctaat

SEQ ID NO: 227 - DNA, 23 bp, Artificial Sequence
  upstream primer of SAUR05
agtgatgatgttcctaagattgt

SEQ ID NO: 228 - DNA, 24 bp, Artificial Sequence
  downstream primer of SAUR05
ggcttagagtataccaaaataccg

SEQ ID NO: 229 - DNA, 27 bp, Artificial Sequence
  upstream primer of SAUR06
ccaatgtgggatgaatcaagaagcatt

SEQ ID NO: 230 - DNA, 23 bp, Artificial Sequence
  downstream primer of SAUR06
tcaatcacagttcgtggctgttc

SEQ ID NO: 231 - DNA, 22 bp, Artificial Sequence
  upstream primer of SP081
gttgagcgtatgaatcctatat

SEQ ID NO: 232 - DNA, 31 bp, Artificial Sequence
  downstream primer of SP081
atggaggactatagaaagagaaaacgaaagt

SEQ ID NO: 233 - DNA, 22 bp, Artificial Sequence
  upstream primer of SZ8824
gctatgtttagggacaggaagt

SEQ ID NO: 234 - DNA, 22 bp, Artificial Sequence
  downstream primer of SZ8824
tgagatcacattaacatgtagc

SEQ ID NO: 235 - DNA, 23 bp, Artificial Sequence
  upstream primer of TSS232
acatgtacgattcatgggcaacc

SEQ ID NO: 236 - DNA, 25 bp, Artificial Sequence
  downstream primer of TSS232
gtgcaaaggtgaacacaggacaaca

SEQ ID NO: 237 - DNA, 24 bp, Artificial Sequence
  upstream primer of C400
cgagtcagtttaggacccctatat

SEQ ID NO: 238 - DNA, 23 bp, Artificial Sequence
  downstream primer of C400
catcatgtccacaagaaccctaa

SEQ ID NO: 239 - DNA, 38 bp, Artificial Sequence
  upstream primer of TSS233
cttggtattatttactttacgttttattttgtctttat

SEQ ID NO: 240 - DNA, 29 bp, Artificial Sequence
  downstream primer of TSS233
tttacaaccaaaatcattgcagatgcggt

SEQ ID NO: 241 - DNA, 27 bp, Artificial Sequence
  upstream primer of TSS234
cactcgctgcatcttaaactattatga

SEQ ID NO: 242 - DNA, 27 bp, Artificial Sequence
  downstream primer of TSS234
ttgtgaggggatcatatctgtagtcgt

SEQ ID NO: 243 - DNA, 27 bp, Artificial Sequence
  upstream primer of TSS235
actacacaaatgggatgagactttcca

SEQ ID NO: 244 - DNA, 25 bp, Artificial Sequence
  downstream primer of TSS235
tacagagaccttcttctcataacca

SEQ ID NO: 245 - DNA, 23 bp, Artificial Sequence
  upstream primer of TSS236
gagttcgcgtattctacaattat

SEQ ID NO: 246 - DNA, 26 bp, Artificial Sequence
  downstream primer of TSS236
aaagtctttacatctccgcatgaatc

SEQ ID NO: 247 - DNA, 26 bp, Artificial Sequence
  upstream primer of TSS237
ttcctaagaaaccctagcttccattt

SEQ ID NO: 248 - DNA, 26 bp, Artificial Sequence
  downstream primer of TSS237
gatttgttcagaccagtatgttcctt

SEQ ID NO: 249 - DNA, 22 bp, Artificial Sequence
  upstream primer of TSS238
caaaatttctccttcaaactca

SEQ ID NO: 250 - DNA, 26 bp, Artificial Sequence
  downstream primer of TSS238
atgtgcctacaagaaattaaatgggt

SEQ ID NO: 251 - DNA, 27 bp, Artificial Sequence
  upstream primer of f10_1149
gcgttgtcttccatgattttcccaagt

SEQ ID NO: 252 - DNA, 23 bp, Artificial Sequence
  downstream primer of f10_1149
aattttggccagaatacgaacaa

SEQ ID NO: 253 - DNA, 25 bp, Artificial Sequence
  upstream primer of TSS240
tcgcttttagccaacactaattatt

SEQ ID NO: 254 - DNA, 24 bp, Artificial Sequence
  downstream primer of TSS240
agaggaagaagaaaacatggggta

SEQ ID NO: 255 - DNA, 27 bp, Artificial Sequence
  upstream primer of TSS241
aatagctagacaatttaatgagtttgt

SEQ ID NO: 256 - DNA, 27 bp, Artificial Sequence
  downstream primer of TSS241
aattccatctcaagtgtcagttgccaa

SEQ ID NO: 257 - DNA, 24 bp, Artificial Sequence
  upstream primer of TSS242
atgagtgagcgtttggttttccgt

SEQ ID NO: 258 - DNA, 30 bp, Artificial Sequence
  downstream primer of TSS242
ccgcagcatcaatcataattatatgcaact

SEQ ID NO: 259 - DNA, 29 bp, Artificial Sequence
  upstream primer of ZZZ162
acaaatatctctcgcatgtctttgtaatt

SEQ ID NO: 260 - DNA, 27 bp, Artificial Sequence
  downstream primer of ZZZ162
gttctctctttaatttcctggtgttgc

SEQ ID NO: 261 - DNA, 27 bp, Artificial Sequence
  upstream primer of TSS245
aggttccaaatttaaatctccctcgca

SEQ ID NO: 262 - DNA, 25 bp, Artificial Sequence
  downstream primer of TSS245
gcgcagtgtaagaggaaaatgttga

SEQ ID NO: 263 - DNA, 31 bp, Artificial Sequence
  upstream primer of SU201
aagaccaactagagtggatagctaactttaa

SEQ ID NO: 264 - DNA, 24 bp, Artificial Sequence
  downstream primer of SU201
tccataaaaaggacacaagacctg

SEQ ID NO: 265 - DNA, 26 bp, Artificial Sequence
  upstream primer of color852
gtgggctcatataaacaacaaataag

SEQ ID NO: 266 - DNA, 24 bp, Artificial Sequence
  downstream primer of color852
accactaaatgtttgatccttgtc

SEQ ID NO: 267 - DNA, 31 bp, Artificial Sequence
  upstream primer of neww26
atctgctacagagaaaaacggtaaaccatct

SEQ ID NO: 268 - DNA, 22 bp, Artificial Sequence
  downstream primer of neww26
cacttgtgcaattcaaaatttc

SEQ ID NO: 269 - DNA, 26 bp, Artificial Sequence
  upstream primer of wwbb1159
aattatcttgccgagctttttgtcct

SEQ ID NO: 270 - DNA, 30 bp, Artificial Sequence
  downstream primer of wwbb1159
ccctagctcctttcgacccttttaatgacc

SEQ ID NO: 271 - DNA, 26 bp, Artificial Sequence
  upstream primer of wwbb1277
tctgttctgtctcctagtttgtatgg

SEQ ID NO: 272 - DNA, 22 bp, Artificial Sequence
  downstream primer of wwbb1277
agagagtgaaggtagctccatc

SEQ ID NO: 273 - DNA, 23 bp, Artificial Sequence
  upstream primer of wwbb1332
gtttctagtgtatgggccaactc

SEQ ID NO: 274 - DNA, 21 bp, Artificial Sequence
  downstream primer of wwbb1332
acgataatgtaactcagctca

SEQ ID NO: 275 - DNA, 31 bp, Artificial Sequence
  upstream primer of wwbb1200
taggaggttcaaatttgagatatttttcaac

SEQ ID NO: 276 - DNA, 26 bp, Artificial Sequence
  downstream primer of wwbb1200
tcatgagagtcttgtatagggttggt

SEQ ID NO: 277 - DNA, 26 bp, Artificial Sequence
  upstream primer of wwbb1373
ctaaaaaccataccagtgaaccaacc

SEQ ID NO: 278 - DNA, 26 bp, Artificial Sequence
  downstream primer of wwbb1373
aattttttcacaccgtttgaactcga

SEQ ID NO: 279 - DNA, 24 bp, Artificial Sequence
  upstream primer of S1122
tgcagacacaacagtaagttctcc

SEQ ID NO: 280 - DNA, 27 bp, Artificial Sequence
  downstream primer of S1122
ctggaatgtgtaccaaaatctcaaata

SEQ ID NO: 281 - DNA, 32 bp, Artificial Sequence
  upstream primer of Zzwy102
tttcaccaatggtcttatattcagaaagacta

SEQ ID NO: 282 - DNA, 20 bp, Artificial Sequence
  downstream primer of Zzwy102
gagaattcccctattttgaa

SEQ ID NO: 283 - DNA, 26 bp, Artificial Sequence
  upstream primer of Hzwy486
caaccaccatccgtgaacatataaac

SEQ ID NO: 284 - DNA, 28 bp, Artificial Sequence
  downstream primer of Hzwy486
aaagggacccaatattttgtttgtttgt

SEQ ID NO: 285 - DNA, 28 bp, Artificial Sequence
  upstream primer of SP101
caacagaatgtccgttagaactcagatt

SEQ ID NO: 286 - DNA, 27 bp, Artificial Sequence
  downstream primer of SP101
atgagtatgcagacgggaaagaactag

SEQ ID NO: 287 - DNA, 26 bp, Artificial Sequence
  upstream primer of wwbb378
gtgagagtgatggtcaagtgataaga

SEQ ID NO: 288 - DNA, 24 bp, Artificial Sequence
  downstream primer of wwbb378
tgctacattcccccaaatttagtt

SEQ ID NO: 289 - DNA, 29 bp, Artificial Sequence
  upstream primer of wwbb411
caaccaaatcaatgaacaagggaagaata

SEQ ID NO: 290 - DNA, 24 bp, Artificial Sequence
  downstream primer of wwbb411
aacgtgatcatgcatctgttgaag

SEQ ID NO: 291 - DNA, 28 bp, Artificial Sequence
  upstream primer of HB123
tgaaaaaacatgaaagtcgcatgaatgt

SEQ ID NO: 292 - DNA, 26 bp, Artificial Sequence
  downstream primer of HB123
gcgccttatacaagtgttatatggtg

SEQ ID NO: 293 - DNA, 24 bp, Artificial Sequence
  upstream primer of newdy33
tgaagagacgtaaggatgcctcac

SEQ ID NO: 294 - DNA, 25 bp, Artificial Sequence
  downstream primer of newdy33
ctcaagtgttgcgaagatgatacag

SEQ ID NO: 295 - DNA, 23 bp, Artificial Sequence
  upstream primer of SZ1348
aacgtaaatttgtttggtttgag

SEQ ID NO: 296 - DNA, 31 bp, Artificial Sequence
  downstream primer of SZ1348
gaacaataacttgcctgaggaaaagtctcca

SEQ ID NO: 297 - DNA, 26 bp, Artificial Sequence
  upstream primer of color1245
cctcatggagattcatttcccttcct

SEQ ID NO: 298 - DNA, 30 bp, Artificial Sequence
  downstream primer of color1245
aacgagctggtgtaagtttcctataaatgg

SEQ ID NO: 299 - DNA, 26 bp, Artificial Sequence
  upstream primer of SZ832
ctgagagacttggccaagaaatacgg

SEQ ID NO: 300 - DNA, 26 bp, Artificial Sequence
  downstream primer of SZ832
ggtgcaaagccaatgtttgtatcacc

SEQ ID NO: 301 - DNA, 21 bp, Artificial Sequence
  upstream primer of SZ906
agaagatacgcggtttgtttt

SEQ ID NO: 302 - DNA, 29 bp, Artificial Sequence
  downstream primer of SZ906
aaaagtcaccccttaaattggaagaggcg

SEQ ID NO: 303 - DNA, 30 bp, Artificial Sequence
  upstream primer of wwbb1441
acaagactaccatagacccccttatgcttc

SEQ ID NO: 304 - DNA, 28 bp, Artificial Sequence
  downstream primer of wwbb1441
acttgaaaggcgggaaattgtatttgag

SEQ ID NO: 305 - DNA, 31 bp, Artificial Sequence
  upstream primer of Y323
caaacgtgaaaatctgaggataagttccctg

SEQ ID NO: 306 - DNA, 27 bp, Artificial Sequence
  downstream primer of Y323
tggggttgttctaatgtaatctgcagc

SEQ ID NO: 307 - DNA, 28 bp, Artificial Sequence
  upstream primer of LY591
cactgcgtgcatatacatacattcatta

SEQ ID NO: 308 - DNA, 27 bp, Artificial Sequence
  downstream primer of LY591
tattatttcttagattcacaggtttcg

SEQ ID NO: 309 - DNA, 30 bp, Artificial Sequence
  upstream primer of SZ4250
caaggagttcctttcattctcatccaaccc

SEQ ID NO: 310 - DNA, 24 bp, Artificial Sequence
  downstream primer of SZ4250
actgaagtgttgctgaagaatgag

SEQ ID NO: 311 - DNA, 25 bp, Artificial Sequence
  upstream primer of Y397
ggggtgttcacataggatttgtatg

SEQ ID NO: 312 - DNA, 21 bp, Artificial Sequence
  downstream primer of Y397
agaacaacctctttcatggca

SEQ ID NO: 313 - DNA, 26 bp, Artificial Sequence
  upstream primer of SZ4378
agtgatacttgccatgtaggactctc

SEQ ID NO: 314 - DNA, 23 bp, Artificial Sequence
  downstream primer of SZ4378
tgtgtccaacgagattaggtgtg

SEQ ID NO: 315 - DNA, 31 bp, Artificial Sequence
  upstream primer of S1246
gcatctagtccattcgtaatgtaggcgccaa

SEQ ID NO: 316 - DNA, 29 bp, Artificial Sequence
  downstream primer of S1246
gaggtcttatcatcctgtccatctccaga

SEQ ID NO: 317 - DNA, 25 bp, Artificial Sequence
  upstream primer of zxx51
gcaaatctttgtattctgcttgcag

SEQ ID NO: 318 - DNA, 22 bp, Artificial Sequence
  downstream primer of zxx51
cttacccttggtttgtactgtt

SEQ ID NO: 319 - DNA, 23 bp, Artificial Sequence
  upstream primer of SZ4457
agttgtatcaaagtcacgaatca

SEQ ID NO: 320 - DNA, 27 bp, Artificial Sequence
  downstream primer of SZ4457
aacacatggaaagagattgatcacctt

SEQ ID NO: 321 - DNA, 25 bp, Artificial Sequence
  upstream primer of SZ4595
gtaataaaaggaggaagtggctgcg

SEQ ID NO: 322 - DNA, 29 bp, Artificial Sequence
  downstream primer of SZ4595
cattaaatgggtggatgaggatgaccccg

SEQ ID NO: 323 - DNA, 31 bp, Artificial Sequence
  upstream primer of neww5
tgtgaccagaccaaacggaaaaactctagat

SEQ ID NO: 324 - DNA, 32 bp, Artificial Sequence
  downstream primer of neww5
atgggtcttgaaatattaatgagattccgttt

SEQ ID NO: 325 - DNA, 26 bp, Artificial Sequence
  upstream primer of CY1821
cggcctatttttgctaatggtatgct

SEQ ID NO: 326 - DNA, 28 bp, Artificial Sequence
  downstream primer of CY1821
gaaagtatgaaatggaaccagtctttaa

SEQ ID NO: 327 - DNA, 20 bp, Artificial Sequence
  upstream primer of SZ12710
gatagatggaagggggaaaa

SEQ ID NO: 328 - DNA, 28 bp, Artificial Sequence
  downstream primer of SZ12710
agggtgggtatattagcactctcaatta

SEQ ID NO: 329 - DNA, 28 bp, Artificial Sequence
  upstream primer of neww61
tgacaccaaccattcaaaagtgctattg

SEQ ID NO: 330 - DNA, 25 bp, Artificial Sequence
  downstream primer of neww61
ttgcaaatggagagtggtgtagtgt

SEQ ID NO: 331 - DNA, 27 bp, Artificial Sequence
  upstream primer of wr70
gatgtaagcacagtttttgtcgatcca

SEQ ID NO: 332 - DNA, 29 bp, Artificial Sequence
  downstream primer of wr70
ggcttctttttatcaccgagttgaaaacg

SEQ ID NO: 333 - DNA, 23 bp, Artificial Sequence
  upstream primer of neww16
aaaatgacagactcgattgcagc

SEQ ID NO: 334 - DNA, 30 bp, Artificial Sequence
  downstream primer of neww16
agtagtgttactggctctgtatcttgttaa

SEQ ID NO: 335 - DNA, 22 bp, Artificial Sequence
  upstream primer of SZ9100
aaccaagaaacaagtcatagct

SEQ ID NO: 336 - DNA, 24 bp, Artificial Sequence
  downstream primer of SZ9100
attcagttgacatctgcgaaagga

SEQ ID NO: 337 - DNA, 29 bp, Artificial Sequence
  upstream primer of CY2464
cgatttctcgactggttaatcgatcatcg

SEQ ID NO: 338 - DNA, 26 bp, Artificial Sequence
  downstream primer of CY2464
aaatccagatctgaaagcacgagatg

SEQ ID NO: 339 - DNA, 25 bp, Artificial Sequence
  upstream primer of wwbb623
accagtgtaaacccctgggattctg

SEQ ID NO: 340 - DNA, 22 bp, Artificial Sequence
  downstream primer of wwbb623
tctcttccttctgctttctcac

SEQ ID NO: 341 - DNA, 25 bp, Artificial Sequence
  upstream primer of SZ4849
ggtagaaagtttcgatcatggttgg

SEQ ID NO: 342 - DNA, 23 bp, Artificial Sequence
  downstream primer of SZ4849
tcacacgggaacttttctcatct

SEQ ID NO: 343 - DNA, 29 bp, Artificial Sequence
  upstream primer of LY659
cacgatcagaaaagctatctcgtctaaca

SEQ ID NO: 344 - DNA, 23 bp, Artificial Sequence
  downstream primer of LY659
aacccatctgatgataagggtag

SEQ ID NO: 345 - DNA, 28 bp, Artificial Sequence
  upstream primer of wr154
gacagaagtgaaacagaaacatctgcag

SEQ ID NO: 346 - DNA, 26 bp, Artificial Sequence
  downstream primer of wr154
gcaaagttccaaggaaatctgatatg

SEQ ID NO: 347 - DNA, 27 bp, Artificial Sequence
  upstream primer of wwbb944
atgcataatccaaagctgcctgaagtg

SEQ ID NO: 348 - DNA, 26 bp, Artificial Sequence
  downstream primer of wwbb944
ccacaactccaacaataactaaccct

SEQ ID NO: 349 - DNA, 23 bp, Artificial Sequence
  upstream primer of neww19
gccgttgattcctgggtaggtga

SEQ ID NO: 350 - DNA, 25 bp, Artificial Sequence
  downstream primer of neww19
attgccgaaagctttaaagactccg

SEQ ID NO: 351 - DNA, 26 bp, Artificial Sequence
  upstream primer of SZ1413
taccagatacgcataacatagtggga

SEQ ID NO: 352 - DNA, 28 bp, Artificial Sequence
  downstream primer of SZ1413
cttacctgaaatcagtttgcttcatact

SEQ ID NO: 353 - DNA, 26 bp, Artificial Sequence
  upstream primer of SZ4976
ccatttccgggaaggtgattaattgc

SEQ ID NO: 354 - DNA, 23 bp, Artificial Sequence
  downstream primer of SZ4976
gtgtcacaatcttctatcgagat

SEQ ID NO: 355 - DNA, 24 bp, Artificial Sequence
  upstream primer of SZ2020
atagaaaccttgacagccaaagac

SEQ ID NO: 356 - DNA, 29 bp, Artificial Sequence
  downstream primer of SZ2020
gggttcaacacaaaacggtcctatcatta

SEQ ID NO: 357 - DNA, 26 bp, Artificial Sequence
  upstream primer of SZ5112
agttttgtggacgaaattgtcttttt

SEQ ID NO: 358 - DNA, 22 bp, Artificial Sequence
  downstream primer of SZ5112
ataagaaatcgtcccactcttc

SEQ ID NO: 359 - DNA, 26 bp, Artificial Sequence
  upstream primer of SZ9195
aacaatttgcgatttcctgtacttgt

SEQ ID NO: 360 - DNA, 24 bp, Artificial Sequence
  downstream primer of SZ9195
gaattgggatgaggagataagaaa

SEQ ID NO: 361 - DNA, 26 bp, Artificial Sequence
  upstream primer of SZ2250
taacccttgtcataatgcagtgtttc

SEQ ID NO: 362 - DNA, 24 bp, Artificial Sequence
  downstream primer of SZ2250
tgttcatgttgttgtacagcacgc

SEQ ID NO: 363 - DNA, 26 bp, Artificial Sequence
  upstream primer of SZ2270
attagtaagacaccaatgacctcaca

SEQ ID NO: 364 - DNA, 27 bp, Artificial Sequence
  downstream primer of SZ2270
gaagcatttgtaatcagtccgacattc

SEQ ID NO: 365 - DNA, 27 bp, Artificial Sequence
  upstream primer of SZ2365
agttggaaatcctttttcatccccttt

SEQ ID NO: 366 - DNA, 27 bp, Artificial Sequence
  downstream primer of SZ2365
tcgcacatcccatcagtttcaatctga

SEQ ID NO: 367 - DNA, 28 bp, Artificial Sequence
  upstream primer of zwyx1188
ctacatctgatgactacagcttgtgcat

SEQ ID NO: 368 - DNA, 23 bp, Artificial Sequence
  downstream primer of zwyx1188
catgttttccattggatctggat

SEQ ID NO: 369 - DNA, 23 bp, Artificial Sequence
  upstream primer of Yzwy320
attcatctcgtgcctatcaactt

SEQ ID NO: 370 - DNA, 28 bp, Artificial Sequence
  downstream primer of Yzwy320
tgaagacaagggagatttaacacattaa

SEQ ID NO: 371 - DNA, 22 bp, Artificial Sequence
  upstream primer of wwbb1139
cgactcatccatccgattataa

SEQ ID NO: 372 - DNA, 29 bp, Artificial Sequence
  downstream primer of wwbb1139
gatccaagaatttaacgactccacaacca

SEQ ID NO: 373 - DNA, 24 bp, Artificial Sequence
  upstream primer of SZ9364
aattgttttcataatactcctctc

SEQ ID NO: 374 - DNA, 28 bp, Artificial Sequence
  downstream primer of SZ9364
gcttttcgaggatccacttgaattttta

SEQ ID NO: 375 - DNA, 29 bp, Artificial Sequence
  upstream primer of SZ2614
ctagccaatagagaccgatgaacaattat

SEQ ID NO: 376 - DNA, 25 bp, Artificial Sequence
  downstream primer of SZ2614
gaaaatttgggagttcaacctgtcg

SEQ ID NO: 377 - DNA, 32 bp, Artificial Sequence
  upstream primer of TSS24702
caaggaatatagcaacgcttgaagaacaagac

SEQ ID NO: 378 - DNA, 24 bp, Artificial Sequence
  downstream primer of TSS24702
attggcccaatttcgtggtgtttg

SEQ ID NO: 379 - DNA, 22 bp, Artificial Sequence
  upstream primer of TSS248
agtgggcctaaaaataaagctt

SEQ ID NO: 380 - DNA, 33 bp, Artificial Sequence
  downstream primer of TSS248
caaaggctgtaaaaagtgtacaaaaatcattta

SEQ ID NO: 381 - DNA, 22 bp, Artificial Sequence
  upstream primer of SZ2733
aacaacccctatgccatgaact

SEQ ID NO: 382 - DNA, 31 bp, Artificial Sequence
  downstream primer of SZ2733
cgaagaggaacagcataagagtgtaccaaat

SEQ ID NO: 383 - DNA, 26 bp, Artificial Sequence
  upstream primer of SZ2805
gtggaattggtggagctctgcataca

SEQ ID NO: 384 - DNA, 26 bp, Artificial Sequence
  downstream primer of SZ2805
cagaaaagaaggtgggaaacttactc

SEQ ID NO: 385 - DNA, 22 bp, Artificial Sequence
  upstream primer of SZ2888
ttcggggtcgtgagaatgttat

SEQ ID NO: 386 - DNA, 26 bp, Artificial Sequence
  downstream primer of SZ2888
ggtatgattaaactgacggttccctc

SEQ ID NO: 387 - DNA, 30 bp, Artificial Sequence
  upstream primer of SZ2987
agtaggtttggcagattgactttgaaatcg

SEQ ID NO: 388 - DNA, 21 bp, Artificial Sequence
  downstream primer of SZ2987
agtgagttgtttttggaccaa

SEQ ID NO: 389 - DNA, 27 bp, Artificial Sequence
  upstream primer of zwyx1207
ttgtcccgtaagtcaacagattcaaat

SEQ ID NO: 390 - DNA, 27 bp, Artificial Sequence
  downstream primer of zwyx1207
gactggaaaacacattaaacagcatta

SEQ ID NO: 391 - DNA, 23 bp, Artificial Sequence
  upstream primer of CY1709
ttcagtgtactgccgaattgaag

SEQ ID NO: 392 - DNA, 27 bp, Artificial Sequence
  downstream primer of CY1709
gtcttaaactgaagaacacctctgccg

SEQ ID NO: 393 - DNA, 24 bp, Artificial Sequence
  upstream primer of SZ3067
gcaagttaatcatgcagaagtcat

SEQ ID NO: 394 - DNA, 28 bp, Artificial Sequence
  downstream primer of SZ3067
gttcgtccataacctagctctttcttca

SEQ ID NO: 395 - DNA, 26 bp, Artificial Sequence
  upstream primer of SZ5253
tcttgttccttggagattcaaagttg

SEQ ID NO: 396 - DNA, 26 bp, Artificial Sequence
  downstream primer of SZ5253
tatagacatggagggacaatggtgaa

SEQ ID NO: 397 - DNA, 22 bp, Artificial Sequence
  upstream primer of Wzwy255
tacgtcaaaacccagtttgatt

SEQ ID NO: 398 - DNA, 20 bp, Artificial Sequence
  downstream primer of Wzwy255
cagaacccatacgcttcaac

SEQ ID NO: 399 - DNA, 29 bp, Artificial Sequence
  upstream primer of S981
aggggaagcttattttcttagggaggttg

SEQ ID NO: 400 - DNA, 30 bp, Artificial Sequence
  downstream primer of S981
gttaagcgtttgaatctgatcaactgttgt

SEQ ID NO: 401 - DNA, 31 bp, Artificial Sequence
  upstream primer of S1005
ggccttctcaagttcttctgctgtgaagatt

SEQ ID NO: 402 - DNA, 28 bp, Artificial Sequence
  downstream primer of S1005
taatgggaagctcttggttgtatttggg

SEQ ID NO: 403 - DNA, 27 bp, Artificial Sequence
  upstream primer of SZ9471
ttcttttcagttcttaactccacacca

SEQ ID NO: 404 - DNA, 27 bp, Artificial Sequence
  downstream primer of SZ9471
acattacacaatggcttcgagaatgca

SEQ ID NO: 405 - DNA, 25 bp, Artificial Sequence
  upstream primer of dyy88
tggaagttgatggttgaattatcat

SEQ ID NO: 406 - DNA, 27 bp, Artificial Sequence
  downstream primer of dyy88
ttaactcaaattgcaggatggaggtgt

SEQ ID NO: 407 - DNA, 24 bp, Artificial Sequence
  upstream primer of zwyx1080
tttattagaagcatgtatcagcgg

SEQ ID NO: 408 - DNA, 20 bp, Artificial Sequence
  downstream primer of zwyx1080
cttccgttttcctttgttgt

SEQ ID NO: 409 - DNA, 26 bp, Artificial Sequence
  upstream primer of S1202
tagatatacaagattcagcatttttt

SEQ ID NO: 410 - DNA, 29 bp, Artificial Sequence
  downstream primer of S1202
aacggctctaatgatttgattatttgata

SEQ ID NO: 411 - DNA, 24 bp, Artificial Sequence
  upstream primer of zhwy64
tattgctagcactcgagagaattg

SEQ ID NO: 412 - DNA, 28 bp, Artificial Sequence
  downstream primer of zhwy64
tgaaatgaatcgcagtggacaacaatag

SEQ ID NO: 413 - DNA, 26 bp, Artificial Sequence
  upstream primer of s1_1
cccacaaagtaaagtttctcccacaa

SEQ ID NO: 414 - DNA, 30 bp, Artificial Sequence
  downstream primer of s1_1
gaaattggagtttttcagggtttgtgttat

SEQ ID NO: 415 - DNA, 28 bp, Artificial Sequence
  upstream primer of XDY359
attctatgtggtgaaagatgacaagact

SEQ ID NO: 416 - DNA, 25 bp, Artificial Sequence
  downstream primer of XDY359
gtactgtaaaggctcaattaccttg

SEQ ID NO: 417 - DNA, 26 bp, Artificial Sequence
  upstream primer of HB15
agttgtcctggaagtcatcagaccag

SEQ ID NO: 418 - DNA, 22 bp, Artificial Sequence
  downstream primer of HB15
aggtcaaatccttcaaaccaac

SEQ ID NO: 419 - DNA, 29 bp, Artificial Sequence
  upstream primer of SZ1609
ccaactacattgacgattggattgttgaa

SEQ ID NO: 420 - DNA, 26 bp, Artificial Sequence
  downstream primer of SZ1609
ggtttttaatcgggcaaaggtatatt

SEQ ID NO: 421 - DNA, 29 bp, Artificial Sequence
  upstream primer of XDY354
gtcctggctgcgttaacatttttaatagg

SEQ ID NO: 422 - DNA, 21 bp, Artificial Sequence
  downstream primer of XDY354
tccaattcatgttttgtggga

SEQ ID NO: 423 - DNA, 26 bp, Artificial Sequence
  upstream primer of ww19
ccaaacactttagatatcaccctcaa

SEQ ID NO: 424 - DNA, 29 bp, Artificial Sequence
  downstream primer of ww19
acaagagtttgatgcctctacttccaacg

SEQ ID NO: 425 - DNA, 28 bp, Artificial Sequence
  upstream primer of ddy19
ctgcatggaaaagcaaaattcctacaat

SEQ ID NO: 426 - DNA, 27 bp, Artificial Sequence
  downstream primer of ddy19
gagttggttatgttgatgttgagttag

SEQ ID NO: 427 - DNA, 31 bp, Artificial Sequence
  upstream primer of TSS25804
tattatcttcgactcttcctctttgaatata

SEQ ID NO: 428 - DNA, 24 bp, Artificial Sequence
  downstream primer of TSS25804
gatttcgttgtaaggtaaagggaa

SEQ ID NO: 429 - DNA, 25 bp, Artificial Sequence
  upstream primer of TSS249
acaatagccagattattaggtccgg

SEQ ID NO: 430 - DNA, 26 bp, Artificial Sequence
  downstream primer of TSS249
catgtgtcagcttgaattttgtcttt

SEQ ID NO: 431 - DNA, 25 bp, Artificial Sequence
  upstream primer of TSS25809
aaagatcctatagcttccggtcgca

SEQ ID NO: 432 - DNA, 27 bp, Artificial Sequence
  downstream primer of TSS25809
actagtcgatttgagggggaaaggcta

SEQ ID NO: 433 - DNA, 24 bp, Artificial Sequence
  upstream primer of s2_6
tagttttaacgtgcatgcaatctc

SEQ ID NO: 434 - DNA, 30 bp, Artificial Sequence
  downstream primer of s2_6
caatgggatttctcttctccagaacaaatg

SEQ ID NO: 435 - DNA, 29 bp, Artificial Sequence
  upstream primer of TSS250
aagtgggtcacgattgaagttcttatcag

SEQ ID NO: 436 - DNA, 26 bp, Artificial Sequence
  downstream primer of TSS250
attcttgaatccagcaatccaaatac

SEQ ID NO: 437 - DNA, 28 bp, Artificial Sequence
  upstream primer of S313
tcaacctcaaccgattatggccatatag

SEQ ID NO: 438 - DNA, 32 bp, Artificial Sequence
  downstream primer of S313
caaagaagggctctcaaagactacaattctag

SEQ ID NO: 439 - DNA, 30 bp, Artificial Sequence
  upstream primer of SZ5388
agaaaagtttctcgagggtttagtgaggga

SEQ ID NO: 440 - DNA, 25 bp, Artificial Sequence
  downstream primer of SZ5388
tcatctcgttttgatggttttcatg

SEQ ID NO: 441 - DNA, 28 bp, Artificial Sequence
  upstream primer of S1252
ggggtgatgtttattgagattgagagct

SEQ ID NO: 442 - DNA, 25 bp, Artificial Sequence
  downstream primer of S1252
tctcatggcttcctccactccaaaa

SEQ ID NO: 443 - DNA, 23 bp, Artificial Sequence
  upstream primer of XDY127
ttaacccaaaagaagagccattc

SEQ ID NO: 444 - DNA, 21 bp, Artificial Sequence
  downstream primer of XDY127
gattcagaaccttccttgcct

SEQ ID NO: 445 - DNA, 26 bp, Artificial Sequence
  upstream primer of SZ9751
aacggtaattaagccagatatttata

SEQ ID NO: 446 - DNA, 24 bp, Artificial Sequence
  downstream primer of SZ9751
agagttaaaatgtgcactggattg

SEQ ID NO: 447 - DNA, 29 bp, Artificial Sequence
  upstream primer of wwbb112
gaaatgataacatgttttggaatggcgta

SEQ ID NO: 448 - DNA, 26 bp, Artificial Sequence
  downstream primer of wwbb112
attttcaaagtttgagggagagaacg

SEQ ID NO: 449 - DNA, 27 bp, Artificial Sequence
  upstream primer of SZ5464
tggactattggggtttgttgatgttgt

SEQ ID NO: 450 - DNA, 22 bp, Artificial Sequence
  downstream primer of SZ5464
cttatgcaacgactagatctgc

SEQ ID NO: 451 - DNA, 24 bp, Artificial Sequence
  upstream primer of S321
attattgctctttcttctttgctg

SEQ ID NO: 452 - DNA, 27 bp, Artificial Sequence
  downstream primer of S321
ccgtacctctctaaatacggaatggag

SEQ ID NO: 453 - DNA, 26 bp, Artificial Sequence
  upstream primer of XDY157
agaattgtttcagaggctaacaccat

SEQ ID NO: 454 - DNA, 24 bp, Artificial Sequence
  downstream primer of XDY157
tgatatgcaccattggttgtcgga

SEQ ID NO: 455 - DNA, 26 bp, Artificial Sequence
  upstream primer of TSS25102
tttttgtcgaagctcacaaattgtgt

SEQ ID NO: 456 - DNA, 24 bp, Artificial Sequence
  downstream primer of TSS25102
attgtcaagcagcaaacgataaat

SEQ ID NO: 457 - DNA, 26 bp, Artificial Sequence
  upstream primer of zwyx925
aaagccaatccaactttacatctgaa

SEQ ID NO: 458 - DNA, 26 bp, Artificial Sequence
  downstream primer of zwyx925
aggtgtgaatttgtcctagttttcta

SEQ ID NO: 459 - DNA, 25 bp, Artificial Sequence
  upstream primer of LYY094
gtggagaactaggtcttttcttgga

SEQ ID NO: 460 - DNA, 26 bp, Artificial Sequence
  downstream primer of LYY094
ctcttacatgttttactcttcattga

SEQ ID NO: 461 - DNA, 32 bp, Artificial Sequence
  upstream primer of zwyx555
tacaaacttttacatgagacgctccaaataac

SEQ ID NO: 462 - DNA, 31 bp, Artificial Sequence
  downstream primer of zwyx555
atcgcaaattacgtgattttaagcagtcaag

SEQ ID NO: 463 - DNA, 30 bp, Artificial Sequence
  upstream primer of XDY160
gcattaaaaccaaagccttccctgtccgaa

SEQ ID NO: 464 - DNA, 29 bp, Artificial Sequence
  downstream primer of XDY160
atggtggggatgattaagacgtaattaaa

SEQ ID NO: 465 - DNA, 22 bp, Artificial Sequence
  upstream primer of wwbb20
ggtttctctctctccccctatt

SEQ ID NO: 466 - DNA, 26 bp, Artificial Sequence
  downstream primer of wwbb20
tgaacttaccagatcagcaaagagtt

SEQ ID NO: 467 - DNA, 25 bp, Artificial Sequence
  upstream primer of TSS252
gtttggatgcttatggattttggag

SEQ ID NO: 468 - DNA, 30 bp, Artificial Sequence
  downstream primer of TSS252
ttcaggggtacaaaatctagatagcaatgc

SEQ ID NO: 469 - DNA, 29 bp, Artificial Sequence
  upstream primer of TSS253
gatctgaagaccaagacaataacaatttt

SEQ ID NO: 470 - DNA, 24 bp, Artificial Sequence
  downstream primer of TSS253
atataatgtagttgttgatgattg

SEQ ID NO: 471 - DNA, 27 bp, Artificial Sequence
  upstream primer of LYY347
ctcaacgggaagaaaagggtgtttaat

SEQ ID NO: 472 - DNA, 22 bp, Artificial Sequence
  downstream primer of LYY347
acatgcgtctaatgaagctgat

SEQ ID NO: 473 - DNA, 25 bp, Artificial Sequence
  upstream primer of SZ5769
tttcattcgcctaactgagtttggt

SEQ ID NO: 474 - DNA, 26 bp, Artificial Sequence
  downstream primer of SZ5769
attgtacgtggagtttgatggacttt

SEQ ID NO: 475 - DNA, 24 bp, Artificial Sequence
  upstream primer of TSS254
tgtgatgatcgtgggcatttatct

SEQ ID NO: 476 - DNA, 29 bp, Artificial Sequence
  downstream primer of TSS254
aattgtctgcaactactaaacatgacctt

SEQ ID NO: 477 - DNA, 24 bp, Artificial Sequence
  upstream primer of neww441
aatctcctttttcctggtcatcta

SEQ ID NO: 478 - DNA, 24 bp, Artificial Sequence
  downstream primer of neww441
cccttgtactggattccaatgacg

SEQ ID NO: 479 - DNA, 25 bp, Artificial Sequence
  upstream primer of color1450
cttcttcacatagccttgtttccat

SEQ ID NO: 480 - DNA, 30 bp, Artificial Sequence
  downstream primer of color1450
tgaggtacggaattaagtcatatggcacgt

SEQ ID NO: 481 - DNA, 25 bp, Artificial Sequence
  upstream primer of SZ5929
agctttcttggcaaggaataatctt

SEQ ID NO: 482 - DNA, 27 bp, Artificial Sequence
  downstream primer of SZ5929
gtctgacagcttgattttgctatgaat

SEQ ID NO: 483 - DNA, 20 bp, Artificial Sequence
  upstream primer of TSS255
gcttgcaggagggccctacc

SEQ ID NO: 484 - DNA, 21 bp, Artificial Sequence
  downstream primer of TSS255
tcaaaccccgacgtttgcatc

SEQ ID NO: 485 - DNA, 25 bp, Artificial Sequence
  upstream primer of wwbb181
gaggaagaaaaaggggaggaaagcc

SEQ ID NO: 486 - DNA, 25 bp, Artificial Sequence
  downstream primer of wwbb181
ttcatagcctcctgccaaatagtgc

SEQ ID NO: 487 - DNA, 29 bp, Artificial Sequence
  upstream primer of SZ6076
ctctgttttggctagtgaaacaagtaaga

SEQ ID NO: 488 - DNA, 23 bp, Artificial Sequence
  downstream primer of SZ6076
atgcatggagaaaagttttccct

SEQ ID NO: 489 - DNA, 28 bp, Artificial Sequence
  upstream primer of neww146
ttgttcttgtccaaaaatcacagcagct

SEQ ID NO: 490 - DNA, 25 bp, Artificial Sequence
  downstream primer of neww146
actttgaatggggattttgaggtgg

SEQ ID NO: 491 - DNA, 22 bp, Artificial Sequence
  upstream primer of SZ273
aacatactctaaggtacgctcc

SEQ ID NO: 492 - DNA, 23 bp, Artificial Sequence
  downstream primer of SZ273
ggatggatcggttttggagataa

SEQ ID NO: 493 - DNA, 27 bp, Artificial Sequence
  upstream primer of XDY231
ctccaagccctaaggatggtattgacc

SEQ ID NO: 494 - DNA, 18 bp, Artificial Sequence
  downstream primer of XDY231
aaaagtccacgtgtcatg

SEQ ID NO: 495 - DNA, 26 bp, Artificial Sequence
  upstream primer of SZ6268
cattttcccgatgctttatgtaatca

SEQ ID NO: 496 - DNA, 29 bp, Artificial Sequence
  downstream primer of SZ6268
gacctttactgttactaacccctgattta

SEQ ID NO: 497 - DNA, 28 bp, Artificial Sequence
  upstream primer of S349
ttaatattgacgccagtagaatggacca

SEQ ID NO: 498 - DNA, 28 bp, Artificial Sequence
  downstream primer of S349
acgccatcaataacgggtatgattcatg

SEQ ID NO: 499 - DNA, 26 bp, Artificial Sequence
  upstream primer of xccc60
tctccctacactacacacttttgttg

SEQ ID NO: 500 - DNA, 29 bp, Artificial Sequence
  downstream primer of xccc60
gaagctctcgtgtttttatcttgcatttt

SEQ ID NO: 501 - DNA, 24 bp, Artificial Sequence
  upstream primer of SZ10554
caaaacagattagatcgattgggt

SEQ ID NO: 502 - DNA, 25 bp, Artificial Sequence
  downstream primer of SZ10554
gcataatatacaaacacttcactgc

SEQ ID NO: 503 - DNA, 30 bp, Artificial Sequence
  upstream primer of TSS260
tatggcacaagcttgtatataatgtcacat

SEQ ID NO: 504 - DNA, 28 bp, Artificial Sequence
  downstream primer of TSS260
gctgaattcatagctcattagtcaatac

SEQ ID NO: 505 - DNA, 23 bp, Artificial Sequence
  upstream primer of TSS261
tgcattttcctctgtgtatgtgt

SEQ ID NO: 506 - DNA, 28 bp, Artificial Sequence
  downstream primer of TSS261
gtatattcgacgtacaaaagatcctcca

SEQ ID NO: 507 - DNA, 25 bp, Artificial Sequence
  upstream primer of SZ6459
ttaattaagccttatttgctgggtg

SEQ ID NO: 508 - DNA, 22 bp, Artificial Sequence
  downstream primer of SZ6459
ttgcaaacttgggatccatatg

SEQ ID NO: 509 - DNA, 21 bp, Artificial Sequence
  upstream primer of SZ10597
aaagcatcgaaaacaaaatcg

SEQ ID NO: 510 - DNA, 27 bp, Artificial Sequence
  downstream primer of SZ10597
aaatccacgcaatatcaaatagcgtgg

SEQ ID NO: 511 - DNA, 27 bp, Artificial Sequence
  upstream primer of TSS263
agtgattagctagtttcatggcttacg

SEQ ID NO: 512 - DNA, 28 bp, Artificial Sequence
  downstream primer of TSS263
ggaaggtcttgaatgtctatgggtcttt

SEQ ID NO: 513 - DNA, 27 bp, Artificial Sequence
  upstream primer of XDY302
actctccctatgtggtctttcaaaatt

SEQ ID NO: 514 - DNA, 25 bp, Artificial Sequence
  downstream primer of XDY302
tttgttcaccaactttacaccatcc

SEQ ID NO: 515 - DNA, 33 bp, Artificial Sequence
  upstream primer of SZ6675
caaacaagacaaagattccattttcccaatcac

SEQ ID NO: 516 - DNA, 22 bp, Artificial Sequence
  downstream primer of SZ6675
ggcgatggagacggaggtccaa

SEQ ID NO: 517 - DNA, 23 bp, Artificial Sequence
  upstream primer of wwbb244
tacccatttgccaatcgttgatt

SEQ ID NO: 518 - DNA, 25 bp, Artificial Sequence
  downstream primer of wwbb244
aaacccagaaggaaatttaaacaaa

SEQ ID NO: 519 - DNA, 23 bp, Artificial Sequence
  upstream primer of BDY17
ctatgaaagtgagagctttttgt

SEQ ID NO: 520 - DNA, 27 bp, Artificial Sequence
  downstream primer of BDY17
gcttaatcgaattgagtcgctaaccca

SEQ ID NO: 521 - DNA, 24 bp, Artificial Sequence
  upstream primer of SZ6815
agtagcaagcttctgtgcaaaaca

SEQ ID NO: 522 - DNA, 25 bp, Artificial Sequence
  downstream primer of SZ6815
tcctctaaacatctccacctgattg

SEQ ID NO: 523 - DNA, 32 bp, Artificial Sequence
  upstream primer of SZ6840
aaatgatcaaagacgtatccacaagacaacta

SEQ ID NO: 524 - DNA, 21 bp, Artificial Sequence
  downstream primer of SZ6840
ctccaatcatcggtgtgaaaa

SEQ ID NO: 525 - DNA, 26 bp, Artificial Sequence
  upstream primer of NCVXX77
actgtttgggatttgtcttccacata

SEQ ID NO: 526 - DNA, 27 bp, Artificial Sequence
  downstream primer of NCVXX77
agtcagggtctgtgtatgacttaaaat

SEQ ID NO: 527 - DNA, 29 bp, Artificial Sequence
  upstream primer of TSS264
ggcttacagcgaatttcagtaacacctgt

SEQ ID NO: 528 - DNA, 28 bp, Artificial Sequence
  downstream primer of TSS264
attgtttgaatttcctagacgctctcgt

SEQ ID NO: 529 - DNA, 24 bp, Artificial Sequence
  upstream primer of SZ7016
gtttttaaatccaaaaaggccaca

SEQ ID NO: 530 - DNA, 21 bp, Artificial Sequence
  downstream primer of SZ7016
atggggaagagagcaatttct

SEQ ID NO: 531 - DNA, 24 bp, Artificial Sequence
  upstream primer of SZ7070
cttggtatacctacgttcttgtat

SEQ ID NO: 532 - DNA, 22 bp, Artificial Sequence
  downstream primer of SZ7070
gtattgctccttgatatctgtt

SEQ ID NO: 533 - DNA, 32 bp, Artificial Sequence
  upstream primer of XDY315
atgcaatgtgcttcaacatcttctcactcagg

SEQ ID NO: 534 - DNA, 26 bp, Artificial Sequence
  downstream primer of XDY315
aacgactttctaaggttttgatgctc

SEQ ID NO: 535 - DNA, 28 bp, Artificial Sequence
  upstream primer of S1234
atcaaattaatgggagctgagtgatgac

SEQ ID NO: 536 - DNA, 26 bp, Artificial Sequence
  downstream primer of S1234
cttcagctcttgaaagactccaaatg

SEQ ID NO: 537 - DNA, 23 bp, Artificial Sequence
  upstream primer of SZ11309
tcaaaatcctaattgaatgcacc

SEQ ID NO: 538 - DNA, 28 bp, Artificial Sequence
  downstream primer of SZ11309
ttagtggtttacactttacactacatgc

SEQ ID NO: 539 - DNA, 27 bp, Artificial Sequence
  upstream primer of GWB581
ccacatttgataatccaattcgtattt

SEQ ID NO: 540 - DNA, 28 bp, Artificial Sequence
  downstream primer of GWB581
ctttcagagttcatgcaaaggttcttca

SEQ ID NO: 541 - DNA, 29 bp, Artificial Sequence
  upstream primer of XWB137
cttttccattatccaaaccagaccaccct

SEQ ID NO: 542 - DNA, 26 bp, Artificial Sequence
  downstream primer of XWB137
cactcatctgttctcatacgaagcaa

SEQ ID NO: 543 - DNA, 25 bp, Artificial Sequence
  upstream primer of XWB392
ctctaagtaaagagaatcgagctcc

SEQ ID NO: 544 - DNA, 22 bp, Artificial Sequence
  downstream primer of XWB392
gaacctgttttgtctgaggatt

SEQ ID NO: 545 - DNA, 26 bp, Artificial Sequence
  upstream primer of Ma
caacgaaaataaacaggtggtgatcg

SEQ ID NO: 546 - DNA, 20 bp, Artificial Sequence
  downstream primer of Ma
ttttgattcttcaggttcct

SEQ ID NO: 547 - DNA, 27 bp, Artificial Sequence
  upstream primer of SZ3450
tttcgagcacacaaattttgtcaatcg

SEQ ID NO: 548 - DNA, 26 bp, Artificial Sequence
  downstream primer of SZ3450
ttcaagggtccctaaaaatcggggac

SEQ ID NO: 549 - DNA, 28 bp, Artificial Sequence
  upstream primer of S616
ttagtaacaaattcaacgtacccctcta

SEQ ID NO: 550 - DNA, 27 bp, Artificial Sequence
  downstream primer of S616
tattgacacgattgaggtacgagaaga

SEQ ID NO: 551 - DNA, 25 bp, Artificial Sequence
  upstream primer of UDY1
taaaaaaccaagagactgagactct

SEQ ID NO: 552 - DNA, 23 bp, Artificial Sequence
  downstream primer of UDY1
gacgaatttaaaatctttcactc

SEQ ID NO: 553 - DNA, 24 bp, Artificial Sequence
  upstream primer of TSS266
gcaactgcaaaggtgaattcatct

SEQ ID NO: 554 - DNA, 28 bp, Artificial Sequence
  downstream primer of TSS266
catcttctcccttgttctctcccttgcc

SEQ ID NO: 555 - DNA, 24 bp, Artificial Sequence
  upstream primer of FDY190
atctgggtagaaaacgagatccga

SEQ ID NO: 556 - DNA, 21 bp, Artificial Sequence
  downstream primer of FDY190
ttagaggtagaggaggaggtg

SEQ ID NO: 557 - DNA, 25 bp, Artificial Sequence
  upstream primer of TSS267
gattaggccacctgagtttgaatcc

SEQ ID NO: 558 - DNA, 23 bp, Artificial Sequence
  downstream primer of TSS267
gtggaaactagctacttccatct

SEQ ID NO: 559 - DNA, 26 bp, Artificial Sequence
  upstream primer of S641
tgcaaaaccatcttctaagggcttta

SEQ ID NO: 560 - DNA, 28 bp, Artificial Sequence
  downstream primer of S641
ggttgttgctctcccttatcttgaaatt

SEQ ID NO: 561 - DNA, 27 bp, Artificial Sequence
  upstream primer of S652
cccacatgctaatgtttgctccatcaa

SEQ ID NO: 562 - DNA, 24 bp, Artificial Sequence
  downstream primer of S652
acccggacaagaaaatctcagata

SEQ ID NO: 563 - DNA, 26 bp, Artificial Sequence
  upstream primer of TSS268
ttgacacaaaagatattgaggaagga

SEQ ID NO: 564 - DNA, 25 bp, Artificial Sequence
  downstream primer of TSS268
agaggtcaataaatttggggacttc

SEQ ID NO: 565 - DNA, 31 bp, Artificial Sequence
  upstream primer of DYS17
aatattgtagccatttcgtattttcggtacg

SEQ ID NO: 566 - DNA, 31 bp, Artificial Sequence
  downstream primer of DYS17
tcggatttcatctcacttcagctgttcaata

SEQ ID NO: 567 - DNA, 18 bp, Artificial Sequence
  upstream primer of TSS269
cagagaggacggcgaaga

SEQ ID NO: 568 - DNA, 21 bp, Artificial Sequence
  downstream primer of TSS269
tccaacttgcgccactttgtg

SEQ ID NO: 569 - DNA, 29 bp, Artificial Sequence
  upstream primer of S504
aaaactgatatatgcatgagacggcttct

SEQ ID NO: 570 - DNA, 25 bp, Artificial Sequence
  downstream primer of S504
tttggcttgtgctccttttatagta

SEQ ID NO: 571 - DNA, 29 bp, Artificial Sequence
  upstream primer of WBBBB132
aaaactgatatatgcatgagacggcttct

SEQ ID NO: 572 - DNA, 25 bp, Artificial Sequence
  downstream primer of WBBBB132
tttggcttgtgctccttttatagta

SEQ ID NO: 573 - DNA, 27 bp, Artificial Sequence
  upstream primer of XDY346
ggctagacaacttaacaaactccggcc

SEQ ID NO: 574 - DNA, 29 bp, Artificial Sequence
  downstream primer of XDY346
gcagttcatttttcccatcaaaacccatg

SEQ ID NO: 575 - DNA, 24 bp, Artificial Sequence
  upstream primer of newdy292
gatttgtttggttttaagcaggta

SEQ ID NO: 576 - DNA, 28 bp, Artificial Sequence
  downstream primer of newdy292
tgccatatttcgtgtggtgtgttaattt

SEQ ID NO: 577 - DNA, 26 bp, Artificial Sequence
  upstream primer of XWB291
ccatgcattgcctattccctaaattc

SEQ ID NO: 578 - DNA, 31 bp, Artificial Sequence
  downstream primer of XWB291
attgtaattttactccaatgcaatcacatat

SEQ ID NO: 579 - DNA, 30 bp, Artificial Sequence
  upstream primer of SZ3158
attgtagagtagtgccaaaagtgcaaatca

SEQ ID NO: 580 - DNA, 26 bp, Artificial Sequence
  downstream primer of SZ3158
taactgctaatccgaaaattaagcga

SEQ ID NO: 581 - DNA, 28 bp, Artificial Sequence
  upstream primer of Y108
tcctgttcagaacacattggtttttact

SEQ ID NO: 582 - DNA, 30 bp, Artificial Sequence
  downstream primer of Y108
actagcagaagaagaactagaaacctctcc

SEQ ID NO: 583 - DNA, 25 bp, Artificial Sequence
  upstream primer of SZ7418
ggagattcatttgcaaagagggttg

SEQ ID NO: 584 - DNA, 24 bp, Artificial Sequence
  downstream primer of SZ7418
gagatttccttctcacacttttgc

SEQ ID NO: 585 - DNA, 30 bp, Artificial Sequence
  upstream primer of TSS271
atacaatcttttaaaagggtgtcctgcata

SEQ ID NO: 586 - DNA, 27 bp, Artificial Sequence
  downstream primer of TSS271
atgagacttaactcagctcaactcctt

SEQ ID NO: 587 - DNA, 29 bp, Artificial Sequence
  upstream primer of XWB111
ttttaccatttgtatgcgcaatgagattt

SEQ ID NO: 588 - DNA, 29 bp, Artificial Sequence
  downstream primer of XWB111
aaaccccataaccataagctgcatagcca

SEQ ID NO: 589 - DNA, 22 bp, Artificial Sequence
  upstream primer of SZ7516
tctggctccttgtaaatatgag

SEQ ID NO: 590 - DNA, 28 bp, Artificial Sequence
  downstream primer of SZ7516
tgctgtgtcagaggagcagaaaaacata

SEQ ID NO: 591 - DNA, 25 bp, Artificial Sequence
  upstream primer of S402
ctcaatcccttgcactggctctagc

SEQ ID NO: 592 - DNA, 23 bp, Artificial Sequence
  downstream primer of S402
taggaggagtgcaaactttgtca

SEQ ID NO: 593 - DNA, 24 bp, Artificial Sequence
  upstream primer of SZ7624
ttgtgtttaccacctacttatgcg

SEQ ID NO: 594 - DNA, 28 bp, Artificial Sequence
  downstream primer of SZ7624
ttaacgatctagtcaaccacacttcttc

SEQ ID NO: 595 - DNA, 23 bp, Artificial Sequence
  upstream primer of LY770
ttctttctttattcgcgcacatt

SEQ ID NO: 596 - DNA, 32 bp, Artificial Sequence
  downstream primer of LY770
cgagatcgaaagctcagaagaatgacagcact

SEQ ID NO: 597 - DNA, 26 bp, Artificial Sequence
  upstream primer of S412
ctggttcggtgagtttggcaatagag

SEQ ID NO: 598 - DNA, 22 bp, Artificial Sequence
  downstream primer of S412
aatccatctatcaccgaacacg

SEQ ID NO: 599 - DNA, 27 bp, Artificial Sequence
  upstream primer of SZ11912
ttgggaaatatttgttgatgtcattag

SEQ ID NO: 600 - DNA, 22 bp, Artificial Sequence
  downstream primer of SZ11912
tgcaccaccatgagaccaattg

SEQ ID NO: 601 - DNA, 27 bp, Artificial Sequence
  upstream primer of GWB26202
gaggccaaaaccaaagagaaaaatatt

SEQ ID NO: 602 - DNA, 27 bp, Artificial Sequence
  downstream primer of GWB26202
ctatggtggcgcgtggagatatcgctt

SEQ ID NO: 603 - DNA, 25 bp, Artificial Sequence
  upstream primer of LYY3327
aggcgatcctttacgtgatggctct

SEQ ID NO: 604 - DNA, 27 bp, Artificial Sequence
  downstream primer of LYY3327
ccagaaattcgaggatgaagaagcagg

SEQ ID NO: 605 - DNA, 24 bp, Artificial Sequence
  upstream primer of S358
gatccttttaagtacggattttcc

SEQ ID NO: 606 - DNA, 26 bp, Artificial Sequence
  downstream primer of S358
gctgtatttgctgaccgttgatgaat

SEQ ID NO: 607 - DNA, 30 bp, Artificial Sequence
  upstream primer of S369
agttgcgaaattctacaccttctaccactg

SEQ ID NO: 608 - DNA, 25 bp, Artificial Sequence
  downstream primer of S369
gtagaagcttgccagtttgtcgagg

SEQ ID NO: 609 - DNA, 20 bp, Artificial Sequence
  upstream primer of LC06
ctacttaaagcattcaggct

SEQ ID NO: 610 - DNA, 24 bp, Artificial Sequence
  downstream primer of LC06
taaatcttagtgtatgctcaccat

SEQ ID NO: 611 - DNA, 22 bp, Artificial Sequence
  upstream primer of S432
catttcagtattcctgtcgtgg

SEQ ID NO: 612 - DNA, 26 bp, Artificial Sequence
  downstream primer of S432
ctcccaaacttgactttcctcatcag

SEQ ID NO: 613 - DNA, 26 bp, Artificial Sequence
  upstream primer of XL13
gactgtagataccaagtgagtactgt

SEQ ID NO: 614 - DNA, 27 bp, Artificial Sequence
  downstream primer of XL13
ctgaatcatttgattgttttggacaac

SEQ ID NO: 615 - DNA, 24 bp, Artificial Sequence
  upstream primer of S407
tgactcctcggataactctcactt

SEQ ID NO: 616 - DNA, 22 bp, Artificial Sequence
  downstream primer of S407
ttgtgtgtggttggtttacatg

SEQ ID NO: 617 - DNA, 22 bp, Artificial Sequence
  upstream primer of nc22
ctatgtttataactagaaccgg

SEQ ID NO: 618 - DNA, 28 bp, Artificial Sequence
  downstream primer of nc22
aactaaggtcgtttagtcaaccaacgta

SEQ ID NO: 619 - DNA, 26 bp, Artificial Sequence
  upstream primer of S468
aagccccaagttttgtgaacagacac

SEQ ID NO: 620 - DNA, 24 bp, Artificial Sequence
  downstream primer of S468
gtaaataccctgccaactaggtac

SEQ ID NO: 621 - DNA, 26 bp, Artificial Sequence
  upstream primer of TSS272
ggcgatgaagaagtttgtccctctct

SEQ ID NO: 622 - DNA, 27 bp, Artificial Sequence
  downstream primer of TSS272
tagaacaagcacgaactttgttttcga

SEQ ID NO: 623 - DNA, 30 bp, Artificial Sequence
  upstream primer of TSS274
gacatggaagtattttaatagtaaaatgct

SEQ ID NO: 624 - DNA, 29 bp, Artificial Sequence
  downstream primer of TSS274
agaaatccctgtgcataaagggctatata

SEQ ID NO: 625 - DNA, 22 bp, Artificial Sequence
  upstream primer of neww27
ctgctcctatactggcaattta

SEQ ID NO: 626 - DNA, 26 bp, Artificial Sequence
  downstream primer of neww27
ttgttctttgccatcttgtcattcat

SEQ ID NO: 627 - DNA, 25 bp, Artificial Sequence
  upstream primer of neww28
caaggggaaggaacaaaagatatta

SEQ ID NO: 628 - DNA, 30 bp, Artificial Sequence
  downstream primer of neww28
tgcttgtgatgttcatcagaaatgcggtgc

SEQ ID NO: 629 - DNA, 28 bp, Artificial Sequence
  upstream primer of CHL1
ggaagttttgaagcaataagaggcctta

SEQ ID NO: 630 - DNA, 23 bp, Artificial Sequence
  downstream primer of CHL1
gccaaggcacatcaatgtatttt

SEQ ID NO: 631 - DNA, 28 bp, Artificial Sequence
  upstream primer of wwbb289
aggattcaatttggattttcacccacgg

SEQ ID NO: 632 - DNA, 24 bp, Artificial Sequence
  downstream primer of wwbb289
gctttgggttggtaattggctact

SEQ ID NO: 633 - DNA, 23 bp, Artificial Sequence
  upstream primer of LY836
cttctgcaaaaccatattgatag

SEQ ID NO: 634 - DNA, 25 bp, Artificial Sequence
  downstream primer of LY836
ctatatgaggatttcagacagtctg

SEQ ID NO: 635 - DNA, 29 bp, Artificial Sequence
  upstream primer of CA201
gcagcacaaaattcatcacaacaaaatcc

SEQ ID NO: 636 - DNA, 26 bp, Artificial Sequence
  downstream primer of CA201
tgggggttatattgggggttgtgatt

SEQ ID NO: 637 - DNA, 24 bp, Artificial Sequence
  upstream primer of S602
attgttctatgcttctaaccaccg

SEQ ID NO: 638 - DNA, 35 bp, Artificial Sequence
  downstream primer of S602
acaaatttaatttttctagcattacttatgtttag